SEQUENCE LISTING (20 sequences)

SEQ ID NO: 1 - DNA, 1356 bp, unidentified
  source 1..1356: mol_type = unassigned DNA
atgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataa

SEQ ID NO: 2 - protein, 451 aa, unidentified
MSELRAFSAPGKALLAGGYLVLDTKYEAFVVGLSARMHAVAHPYGSLQGSDKFEVRVKSKQFKDGEWLYHISPKSGFIPVSIGGSKNPFIEKVIANVFSYFKPNMDDYCNRNLFVIDIFSDDAYHSQEDSVTEHRGNRRLSFHSHRIEEVPKTGLGSSAGLVTVLTTALASFFVSDLENNVDKYREVIHNLAQVAHCQAQGKIGSGFDVAAAAYGSIRYRRFPPALISNLPDIGSATYGSKLAHLVDEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVITKQDVDLRAQTANDKRFSKVQWLDVTQADWGVRKEKDPETYLDK

SEQ ID NO: 3 - DNA, 1197 bp, unidentified
  source 1..1197: mol_type = unassigned DNA
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 4 - protein, 398 aa, unidentified
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 5 - DNA, 1332 bp, unidentified
  source 1..1332: mol_type = unassigned DNA
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataa

SEQ ID NO: 6 - protein, 443 aa, unidentified
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 7 - DNA, 1476 bp, unidentified
  source 1..1476: mol_type = unassigned DNA
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 8 - protein, 491 aa, unidentified
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 9 - DNA, 3165 bp, unidentified
  source 1..3165: mol_type = unassigned DNA
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagactccaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagtgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaatttttgaattgattttaactcctacattttattctgctatcttagcgcttagactggaaatgaatgttatccacagatctactattatcaagcaaacattagaagaagacggtgttgttccatctacagcaagaatcatttctaaagcagaaaagaaatccgtatcttctttcttaaatctcagtgtggttgtcattatcatgaaactctctgtcatactgttgtttgtcttcatcaacttttataactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattgaatgctgctagaattcataccagttatactgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 10 - protein, 1054 aa, unidentified
MPPLFKGLKQMAKPIAYVSRFSAKRPIHIILFSLIISAFAYLSVIQYYFNGWQLDSNSVFETAPNKDSNTLFQECSHYYRDSSLDGWVSITAHEASELPAPHHYYLLNLNFNSPNETDSIPELANTVFEKDNTKYILQEDLSVSKEISSTDGTKWRLRSDRKSLFDVKTLAYSLYDVFSENVTQADPFDVLIMVTAYLMMFYTIFGLFNDMRKTGSNFWLSASTVVNSASSLFLALYVTQCILGKEVSALTLFEGLPFIVVVVGFKHKIKIAQYALEKFERVGLSKRITTDEIVFESVSEEGGRLIQDHLLCIFAFIGCSMYAHQLKTLTNFCILSAFILIFELILTPTFYSAILALRLEMNVIHRSTIIKQTLEEDGVVPSTARIISKAEKKSVSSFLNLSVVVIIMKLSVILLFVFINFYNFGANWVNDAFNSLYFDKERVSLPDFITSNASENFKEQAIVSVTPLLYYKPIKSYQRIEDMVLLLLRNVSVAIRDRFVSKLVLSALVCSAVINVYLLNAARIHTSYTADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 11 - DNA, 3138 bp, unidentified
  source 1..3138: mol_type = unassigned DNA
atgtcacttcccttaaaaacgatagtacatttggtaaagccctttgcttgcactgctaggtttagtgcgagatacccaatccacgtcattgttgttgctgttttattgagtgccgctgcttatctatccgtgacacaatcttaccttaacgaatggaagctggactctaatcagtattctacatacttaagcataaagccggatgagttgtttgaaaaatgcacacactactataggtctcctgtgtctgatacatggaagttactcagctctaaagaagccgccgatatttataccccttttcattattatttgtctaccataagttttcaaagtaaggacaattcaacgactttgccttcccttgatgacgttatttacagtgttgaccataccaggtacttattaagtgaagagccaaagataccaactgaactagtgtctgaaaacggaacgaaatggagattgagaaacaacagcaattttattttggacctgcataatatttaccgaaatatggtgaagcaattttctaacaaaacgagcgaatttgatcagttcgatttgtttatcatcctagctgcttaccttactcttttttatactctctgttgcctgtttaatgacatgaggaaaatcggatcaaagttttggttaagcttttctgctctttcaaactctgcatgcgcattatatttatcgctgtacacaactcacagtttattgaagaaaccggcttccttattaagtttggtcattggactaccatttatcgtagtaattattggctttaagcataaagttcgacttgcggcattctcgctacaaaaattccacagaattagtattgacaagaaaataacggtaagcaacattatttatgaggctatgtttcaagaaggtgcctacttaatccgcgactacttattttatattagctccttcattggatgtgctatttatgctagacatcttcccggattggtcaatttctgtattttgtctacatttatgctagttttcgacttgcttttgtctgctactttttattctgccattttatcaatgaagctggaaattaacatcattcacagatcaaccgtcatcagacagactttggaagaggacggagttgtcccaactacagcagatattatatataaggatgaaactgcctcagaaccacattttttgagatctaacgtggctatcattctgggaaaagcatcagttattggtcttttgcttctgatcaacctttatgttttcacagataagttaaatgctacaatactaaacacggtatattttgactctacaatttactcgttaccaaattttatcaattataaagatattggcaatctcagcaatcaagtgatcatttccgtgttgccaaagcaatattatactccgctgaaaaaataccatcagatcgaagattctgttctacttatcattgattccgttagcaatgctattcgggaccaatttatcagcaagttacttttttttgcatttgcagttagtatttccatcaatgtctacttactgaatgctgcaaaaattcacacaggatacatgaacttccaaccacaatcaaataagatcgatgatcttgttgttcagcaaaaatcggcaacgattgagttttcagaaactcgaagtatgcctgcttcttctggcctagaaactccagtgaccgcgaaagatataattatctctgaagaaatccagaataacgaatgcgtctatgctttgagttcccaggacgagcctatccgtcctttatcgaatttagtggaacttatggagaaagaacaattaaagaacatgaataatactgaggtttcgaatcttgtcgtcaacggtaaactgccattatattccttagagaaaaaattagaggacacaactcgtgcggttttagttaggagaaaggcactttcaactttggctgaatcgccaattttagtttccgaaaaattgcccttcagaaattatgattatgatcgcgtttttggagcttgctgtgaaaatgtcatcggctatatgccaataccagttggtgtaattggtccattaattattgatggaacatcttatcacataccaatggcaaccacggaaggttgtttagtggcttcagctatgcgtggttgcaaagccatcaatgctggtggtggtgcaacaactgttttaaccaaagatggtatgactagaggcccagtcgttcgtttccctactttaataagatctggtgcctgcaagatatggttagactcggaagagggacaaaattcaattaaaaaagcttttaattctacatcaaggtttgcacgtttgcaacatattcaaacctgtctagcaggcgatttgctttttatgagatttcggacaactaccggtgacgcaatgggtatgaacatgatatcgaaaggtgtcgaatactctttgaaacaaatggtagaagaatatggttgggaagatatggaagttgtctccgtatctggtaactattgtactgataagaaacctgccgcaatcaattggattgaaggtcgtggtaaaagtgtcgtagctgaagctactattcctggtgatgtcgtaaaaagtgttttaaagagcgatgtttccgctttagttgaattaaatatatccaagaacttggttggatccgcaatggctggatctgttggtggtttcaacgcgcacgcagctaatttggtcactgcacttttcttggcattaggccaagatcctgcgcagaacgtcgaaagttccaactgtataactttgatgaaggaagttgatggtgatttaaggatctctgtttccatgccatctattgaagttggtacgattggcgggggtactgttctggagcctcagggcgccatgcttgatcttctcggcgttcgtggtcctcaccccactgaacctggagcaaatgctaggcaattagctagaataatcgcgtgtgctgtcttggctggtgaactgtctctgtgctccgcacttgctgccggtcacctggtacaaagccatatgactcacaaccgtaaaacaaacaaagccaatgaactgccacaaccaagtaacaaagggcccccctgtaaaacctcagcattattataa

SEQ ID NO: 12 - protein, 1045 aa, unidentified
MSLPLKTIVHLVKPFACTARFSARYPIHVIVVAVLLSAAAYLSVTQSYLNEWKLDSNQYSTYLSIKPDELFEKCTHYYRSPVSDTWKLLSSKEAADIYTPFHYYLSTISFQSKDNSTTLPSLDDVIYSVDHTRYLLSEEPKIPTELVSENGTKWRLRNNSNFILDLHNIYRNMVKQFSNKTSEFDQFDLFIILAAYLTLFYTLCCLFNDMRKIGSKFWLSFSALSNSACALYLSLYTTHSLLKKPASLLSLVIGLPFIVVIIGFKHKVRLAAFSLQKFHRISIDKKITVSNIIYEAMFQEGAYLIRDYLFYISSFIGCAIYARHLPGLVNFCILSTFMLVFDLLLSATFYSAILSMKLEINIIHRSTVIRQTLEEDGVVPTTADIIYKDETASEPHFLRSNVAIILGKASVIGLLLLINLYVFTDKLNATILNTVYFDSTIYSLPNFINYKDIGNLSNQVIISVLPKQYYTPLKKYHQIEDSVLLIIDSVSNAIRDQFISKLLFFAFAVSISINVYLLNAAKIHTGYMNFQPQSNKIDDLVVQQKSATIEFSETRSMPASSGLETPVTAKDIIISEEIQNNECVYALSSQDEPIRPLSNLVELMEKEQLKNMNNTEVSNLVVNGKLPLYSLEKKLEDTTRAVLVRRKALSTLAESPILVSEKLPFRNYDYDRVFGACCENVIGYMPIPVGVIGPLIIDGTSYHIPMATTEGCLVASAMRGCKAINAGGGATTVLTKDGMTRGPVVRFPTLIRSGACKIWLDSEEGQNSIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVKSVLKSDVSALVELNISKNLVGSAMAGSVGGFNAHAANLVTALFLALGQDPAQNVESSNCITLMKEVDGDLRISVSMPSIEVGTIGGGTVLEPQGAMLDLLGVRGPHPTEPGANARQLARIIACAVLAGELSLCSALAAGHLVQSHMTHNRKTNKANELPQPSNKGPPCKTSALL

SEQ ID NO: 13 - protein, 527 aa, unidentified
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 14 - protein, 526 aa, unidentified
AAKIHTGYMNFQPQSNKIDDLVVQQKSATIEFSETRSMPASSGLETPVTAKDIIISEEIQNNECVYALSSQDEPIRPLSNLVELMEKEQLKNMNNTEVSNLVVNGKLPLYSLEKKLEDTTRAVLVRRKALSTLAESPILVSEKLPFRNYDYDRVFGACCENVIGYMPIPVGVIGPLIIDGTSYHIPMATTEGCLVASAMRGCKAINAGGGATTVLTKDGMTRGPVVRFPTLIRSGACKIWLDSEEGQNSIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVKSVLKSDVSALVELNISKNLVGSAMAGSVGGFNAHAANLVTALFLALGQDPAQNVESSNCITLMKEVDGDLRISVSMPSIEVGTIGGGTVLEPQGAMLDLLGVRGPHPTEPGANARQLARIIACAVLAGELSLCSALAAGHLVQSHMTHNRKTNKANELPQPSNKGPPCKTSALL

SEQ ID NO: 15 - DNA, 1191 bp, unidentified
  source 1..1191: mol_type = unassigned DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 16 - protein, 396 aa, unidentified
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 17 - DNA, 867 bp, unidentified
  source 1..867: mol_type = unassigned DNA
atgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataa

SEQ ID NO: 18 - protein, 288 aa, unidentified
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 19 - protein, 507 aa, unidentified
DLVVQQKSATIEFSETRSMPASSGLETPVTAKDIIISEEIQNNECVYALSSQDEPIRPLSNLVELMEKEQLKNMNNTEVSNLVVNGKLPLYSLEKKLEDTTRAVLVRRKALSTLAESPILVSEKLPFRNYDYDRVFGACCENVIGYMPIPVGVIGPLIIDGTSYHIPMATTEGCLVASAMRGCKAINAGGGATTVLTKDGMTRGPVVRFPTLIRSGACKIWLDSEEGQNSIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVKSVLKSDVSALVELNISKNLVGSAMAGSVGGFNAHAANLVTALFLALGQDPAQNVESSNCITLMKEVDGDLRISVSMPSIEVGTIGGGTVLEPQGAMLDLLGVRGPHPTEPGANARQLARIIACAVLAGELSLCSALAAGHLVQSHMTHNRKTNKANELPQPSNKGPPCKTSALL

SEQ ID NO: 20 - protein, 197 aa, unidentified
SEEIQNNECVYALSSQDEPIRPLSNLVELMEKEQLKNMNNTEVSNLVVNGKLPLYSLEKKLEDTTRAVLVRRKALSTLAESPILVSEKLPFRNYDYDRVFGACCENVIGYMPIPVGVIGPLIIDGTSYHIPMATTEGCLVASAMRGCKAINAGGGATTVLTKDGMTRGPVVRFPTLIRSGACKIWLDSEEGQNSIKK

(19) United States
(12) Patent Application Publication
(10) Pub. No.: US 2026/0117264 A1
(43) Pub. Date: Apr. 30, 2026

(54) GENETICALLY ENGINEERED MICROBE FOR PRODUCTION OF ISOPRENE FROM A CARBON SOURCE AND METHOD OF PRODUCTION THEREOF

(71) Applicant: CleanJoule, Inc., Salt Lake City, UT (US)

(72) Inventors: Christopher Paul Smith, Bozeman, MT (US); Robert Andrew Price, Salt Lake City, UT (US); Tsokyi Choera, Salt Lake City, UT (US); Younghwan Kim, Salt Lake City, UT (US); Peter Otoupal, Salt Lake City, UT (US)

(73) Assignee: CleanJoule, Inc., Salt Lake City, UT (US)

(21) Appl. No.: 19/374,206

(22) Filed: Oct. 30, 2025

Related U.S. Application Data
(60) Provisional application No. 63/713,932, filed on Oct. 30, 2024.

(51) Int. Cl. C12P 5/00; C12N 1/18; C12N 1/20; C12N 9/00; C12N 9/02; C12N 9/04; C12N 9/88; C12R 1/66; C12R 1/865

(52) U.S. Cl. CPC C12P 5/007; C12N 1/18; C12N 1/20; C12N 9/0006; C12N 9/0008; C12N 9/88; C12N 9/93; C12Y 101/01001; C12Y 102/0101; C12Y 102/01012; C12Y 401/01001; C12Y 402/03027; C12Y 602/01001; C12R 2001/66; C12R 2001/865

(57) ABSTRACT

A genetically engineered microbe capable of producing isoprene from a carbon source and method related thereto include a first nucleic acid sequence encoding a first enzyme, wherein the first enzyme is configured to catalyze one or more steps of a conversion from the carbon source to acetyl coenzyme A (A-CoA), a second nucleic acid sequence encoding a second enzyme of a mevalonate (MVA) pathway, and a heterologous nucleic acid sequence encoding a third enzyme, wherein the third enzyme is configured to catalyzing an isoprene-producing chemical reaction.

### CROSS-REFERENCE TO RELATED APPLICATIONS

This application claims the benefit of priority of U.S. Provisional Patent Application Ser. No. 63/713,932, filed on Oct. 30, 2024, and titled “GENETICALLY ENGINEERED MICROBE FOR PRODUCTION OF ISOPRENE FROM ETHANOL AND METHOD OF PRODUCTION THEREOF,” which is incorporated by reference herein in its entirety.

### FIELD OF THE INVENTION

The present invention generally relates to the field of metabolic engineering. In particular, the present invention is directed to a genetically engineered microbe for production of isoprene from a carbon source.

### REFERENCE TO SEQUENCE LISTING

This specification includes a sequence listing submitted herewith, which includes the file entitled 1656-003USU1.xml having the following size: 37,946 bytes which was created Oct. 6, 2025, the contents of which are incorporated by reference herein.

### BACKGROUND

Isoprene is an important commodity chemical used in a wide range of industrial applications, from the production of synthetic rubber for tires and coatings to uses in adhesives and development of specialty elastomers. Current production of isoprene is derived mostly from unsustainable petrochemical sources and often involves resource-intensive methods that require extensive land use, high temperature, and/or use of precious metals. Biosynthesis of isoprene and related terpenes, while showing some progress, often has a limited yield due to a low carbon efficiency, which may be caused by competing fermentation processes that produce ethanol.

### SUMMARY OF THE DISCLOSURE

In an aspect, a genetically engineered microbe capable of producing isoprene from ethanol is described. The genetically engineered microbe includes a first nucleic acid sequence encoding a first enzyme, wherein the first enzyme is configured to catalyze one or more steps of a conversion from ethanol to acetyl coenzyme A (A-CoA). The genetically engineered microbe further includes a second nucleic acid sequence encoding a second enzyme of a mevalonate (MVA) pathway. The genetically engineered microbe further includes a heterologous nucleic acid sequence encoding a third enzyme, wherein the third enzyme is configured to catalyze an isoprene-producing chemical reaction. In one or more embodiments, the genetically engineered microbe may include a deletion, or attenuation, of an acetyl coenzyme A synthetase (ACS) gene and/or an expression thereof.

In another aspect, a method of producing isoprene from ethanol is described. Method includes culturing a genetically engineered microbe under suitable conditions. The genetically engineered microbe includes first nucleic acid sequence encoding a first enzyme, wherein the first enzyme is configured to catalyze one or more steps of a conversion from ethanol to acetyl coenzyme A (A-CoA), a second nucleic acid sequence encoding a second enzyme of a mevalonate (MVA) pathway, and a heterologous nucleic acid sequence encoding a third enzyme, wherein the third enzyme is configured to catalyze an isoprene-producing chemical reaction. In one or more embodiments, the genetically engineered microbe may include a deletion, or attenuation, of an acetyl coenzyme A synthetase (ACS) gene and/or an expression thereof. The method further includes providing a substrate to the cultured genetically engineered microbe to produce isoprene.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a scheme of an exemplary embodiment of a metabolic pathway that converts ethanol to isoprene, including a plurality of enzymes catalyzing individual steps and representative genes encoding such enzymes;

FIG. 2 is an exemplary scheme containing an overview of metabolic pathways pertaining to biosynthesis of isoprene;

FIGS. 3A-B are additional exemplary schemes of expanded metabolic pathways that are relevant for biosynthesis of isoprene; potential gene targets are also included, wherein the gene targets may be overexpressed or knocked down/knocked out to improve the yield of isoprene;

FIG. 4 is a scheme of an exemplary embodiment of an engineered metabolic pathway pertaining to biosynthesis of isoprene;

FIG. 5 is a scheme of an exemplary embodiment of an engineered metabolic pathway pertaining to biosynthesis of isoprene in yeast;

FIG. 6 is a table of constructed strains of yeast with genetically modification;

FIG. 7 is an alignment of the ISPS protein sequences against the wild type ISPS sequence from P. alba;

FIG. 8 is an exemplary embodiment of experimental results showing isoprene production levels, indicated by raw gas chromatography (GC) traces of samples from all Yest Isoprene Production Screen 2 (YIPS_2) cultures;

FIG. 9 is an exemplary embodiment of experimental results showing a bar graph of final optical density (OD₆₀₀ₙₘ) and corresponding isoprene peak area (pA) for various engineered yeast strains following the YIPS_2 assay;

FIG. 10 is an exemplary embodiment of experimental results showing isoprene production levels, indicated by raw gas chromatography (GC) traces of samples from all Yest Isoprene Production Screen 3 (YIPS_3) cultures;

FIG. 11 is an exemplary embodiment of experimental results showing a bar graph of final optical density (OD₆₀₀ₙₘ) and corresponding isoprene peak area (pA) for various engineered yeast strains following the YIPS_3 assay;

FIG. 12 is an exemplary embodiment of experimental results showing isoprene production levels, indicated by raw gas chromatography (GC) traces of samples from Yest Isoprene Production Screen 4 (YIPS_4) cultures;

FIG. 13 is an exemplary embodiment of experimental results showing a bar graph of final optical density (OD₆₀₀ₙₘ) and corresponding isoprene peak area (pA) for various engineered yeast strains following the YIPS_4 assay;

FIGS. 14A-B are exemplary embodiments of experimental results showing isoprene production levels, indicated by raw gas chromatography (GC) traces of samples from Yest Isoprene Production Screen 4 (YIPS_5) cultures;

FIG. 15 is an exemplary embodiment of experimental results showing a bar graph of final optical density (OD₆₀₀ₙₘ) and corresponding isoprene peak area (pA) for various engineered yeast strains following the YIPS_5 assay;

FIG. 16 is an exemplary embodiment of experimental results showing a bar graph of final optical density (OD₆₀₀ₙₘ) and corresponding isoprene peak area (pA) for various engineered yeast strains following the YIPS_6 assay;

FIG. 17 is an exemplary embodiment of experimental results showing a bar graph of change in carbon (% Vol) for various engineered yeast strains following the YIP_6 assay;

FIG. 18 is a table of a compilation of isoprene peak areas identified across experimental assays from YIPS_002 to YIPS_006;

FIG. 19 is a table of strains and conditions evaluated in experiments YIPS_2-6.

FIGS. 20A-N are exemplary embodiments of plasmid vector maps used for experiments.

FIG. 21 is a scheme of an exemplary embodiment of a native mevalonate (MVA) pathway in yeast with the upstream pathways for synthesis of Acetyl-CoA and downstream pathways for conversion to isoprene by introduction of IspS;

FIG. 22 is an exemplary embodiment of a sequence construct for EutE expression in a yeast strain with a recyclable K. lactic Ura3;

FIG. 23 is an exemplary experiment results of production of strains with or without EutE expression with dextrose or ethanol as the carbon source; and

FIG. 24 is a schematic illustration of an exemplary embodiment of a method for producing isoprene from ethanol using a genetically engineered microbe.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to genetically engineered microbes for production of isoprene from ethanol and methods of production related thereto.

In one or more embodiments, genetically engineered microbe may include bacteria. In some cases, genetically engineered bacteria may include acetic acid bacteria (AAB), such as without limitation Acetobacter including Acetobacter aceti, Acetobacter cerevisiae, Acetobacter malorum, Acetobacter oeni, Acetobacter pasteurianus, Acetobacter pomorum, Acetobacter peroxydans, Acetobacter diazotrophicu, Acetobacter europaeus, Acetobacter hansenii, Acetobacter liquefaciens, Acetobacter xylinus, Acetobacter cibinongensis, Acetobacter estunensis, Acetobacter indonesiensis, Acetobacter nitrogenifigens, Acetobacter orientalis, Acetobacter orleanensis, Acetobacter tropicalis, Acetobacter lovaniensis, and Acetobacter syzygii, Acidomonas, Ameyamaea, Asaia, Bombella, Commensalibacter, Endobacter, Gluconacetobacter including Gluconacetobacter entanii, Gluconacetobacter liquefaciens, Gluconacetobacter azotocaptans, Gluconacetobacter diazotrophicus, Gluconacetobacter sacchari, Gluconacetobacter europaeus, Gluconacetobacter hansenii, Gluconacetobacter intermedius, Gluconacetobacter nataicola, Gluconacetobacter oboediens, Gluconacetobacter rhaeticus, Gluconacetobacter saccharivorans, Gluconacetobacter swingisii, and Gluconacetobacter xylinus, Gluconobacter including Gluconobacter oxydans, Gluconobacter albidus, Gluconobacter cerinus, and Gluconobacter frateurii, Granulibacter, Komagataeibacter including Komagataeibacter europaeus, Komagataeibacter hansenii, Komagataeibacter intermedius, Komagataeibacter medellinensis, Komagataeibacter oboediens, and Komagataeibacter xylinus, Kozakia, Neoasaia, Neokomagataea, Nguyenibacter, Saccharibacter, Swaminathania, Swingsia, and Tanticharoeni Acetobacter.

In some cases, genetically engineered bacteria may include Escherichia including Escherichia Coli (E. coli), Bacillus including Bacillus subtilis (B. subtilis), Clostridium including Clostridium ljungdahlii (C. ljungdahlii), Pseudomonas including Pseudomonas putida (P. putida), and/or Vibrio including Vibrio natriegens (V. natriegens), among others.

In one or more embodiments, genetically engineered microbe may include fungi. In some cases, fungi may include Saccharomyces including Saccharomyces cerevisiae (S. cerevisiae), Aspergillus including Aspergillus niger, Aspergillus nidulans, and/or Aspergillus oryzae, and/or Yarrowia including Yarrowia lipolytica (Y. lipolytica), and/or Ogataea including Ogataea parapolymorpha, Ogataea angusta, Ogataea thermomethanolica among others.

In one or more embodiments, genetically engineered microbe may include a Crabtree-positive microbe. In one or more embodiments, genetically engineered microbe may include a diploid strain.

Genetically engineered microbe includes a first nucleic acid sequence encoding a first enzyme, wherein the first enzyme is configured to catalyze one or more steps of a conversion from ethanol to acetyl coenzyme A (acetyl-CoA or A-CoA).

In one or more embodiments, the first enzyme may include alcohol dehydrogenase (ADH) that converts ethanol to acetaldehyde. In one or more embodiments, the first enzyme may include acetaldehyde dehydrogenase (ALD) that converts acetaldehyde to acetate. In one or more embodiments, the first enzyme may include acetyl coenzyme A synthetase (ACS)/Acetate-CoA ligase that converts acetate to A-CoA. In one or more embodiments, first enzyme may include Acylating Aceta Ldehyde Dehydrogenase (A-ALD) that converts acetaldehyde to A-CoA. In one or more embodiments, acetylating acetaldehyde dehydrogenase (A-ALD) may include the enzyme EutE from Escherichia coli. In one or more embodiments, EutE may include an enzyme that functions in ethanolamine degradation by converting acetaldehyde into acetyl-CoA in an ATP-independent manner. In one or more embodiments, the expression of EutE from E. coli may be incorporated into S. cerevisiae to increase the intracellular acetyl-CoA pool and enhance isoprene production. In one or more embodiments, ethanol may serve as an alternative carbon source for engineered yeast strains expressing EutE, thereby supporting ATP-efficient acetyl-CoA synthesis and increased flux through the mevalonate pathway toward isoprene biosynthesis.

In one or more embodiments, the genetically engineered microbe may have been modified such that the activity of the first enzyme is modulated by increasing an expression of the first nucleic acid sequence.

The genetically engineered microbe further includes a second nucleic acid sequence encoding a second enzyme of a mevalonate (MVA) pathway.

In one or more embodiments, the second enzyme may include acetoacetyl-coenzyme A thiolase or 3-hydroxy-3-methylglutaryl-coenzyme A reductase (MvaE, SEQ ID NO: 4, encoded by ERG10 gene, SEQ ID NO: 3) that converts A-CoA to acetoacetyl-coenzyme A (AA-CoA). In one or more embodiments, the second enzyme may include 3-hydroxy-3-methylglutaryl-CoA synthase (MvaS, SEQ ID NO: 8, encoded by ERG13 gene, SEQ ID NO: 7) that converts AA-CoA to 3-hydroxy-3-methylglutaryl-CoA (HMG-COA). In one or more embodiments, the second enzyme may include MvaE or HMG-COA reductase (HMGR) (SEQ ID NO: 10, encoded by HMG1 gene, SEQ ID NO: 9; SEQ ID NO: 12, encoded by HMG2 gene, SEQ ID NO: 11) that reduces HMG-COA to form mevalonate (Mev). In one or more embodiments, the second enzyme may additionally include a truncated HMG-COA reductase, such as a truncated HMG1 (tHMG1) or truncated HMG2 (tHMG2), wherein at least a portion of the N-terminal membrane anchor domain is removed (SEQ ID NO: 13, encoded by tHMG1 gene, SEQ ID NO: 14, encoded by tHMG2 gene version 1). tHMG1 and/or tHMG2 may reduces HMG-CoA to form mevalonate (Mev). In one or more embodiments, the second enzyme may include mevalonate kinase (MK, SEQ ID NO: 6, encoded by ERG12 gene, SEQ ID NO: 5) that phosphorylates Mev to form mevalonate-5-phosphate (Mev-P or MevP). In one or more embodiments, the second enzyme may include phosphomevalonate kinase (PMK, SEQ ID NO: 2, encoded by ERG8 gene, SEQ ID NO: 1) that phosphorylates Mev-P to form mevalonate-5-diphosphate (Mev-PP or MevPP). In one or more embodiments, the second enzyme may include mevalonate-5-diphosphate decarboxylase or diphosphomevalonate decarboxylase (PMD, SEQ ID: 16, encoded by MVD1 gene, SEQ ID NO: 15) that decarboxylates Mev-PP to produce isopentenyl diphosphate (IPP). In one or more embodiments, the second enzyme may include isopentenyl diphosphate isomerase (IDI or idi, SEQ ID NO: 18, encoded by IDI1 gene, SEQ ID NO: 17) that isomerizes IPP to form dimethylallyl diphosphate (DMAPP). In one or more embodiments, tHMG2 gene may include truncated version 2 (SEQ ID: 19) and truncated version 3 (SEQ ID: 20).

In one or more embodiments, a truncated HMG1 (tHMG1) may include a portion of a N-terminal membrane anchor domain being removed. In one or more embodiments, removed domain may include amino acids from 1 to 530 of the HMG1 sequence; 1 to 531 of the HMG1 sequence; 1 to 532 of the HMG1 sequence; 1 to 533 of the HMG1 sequence; 1 to 534 of the HMG1 sequence; 1 to 535 of the HMG1 sequence; 1 to 536 of the HMG1 sequence; 1 to 537 of the HMG1 sequence; 1 to 538 of the HMG1 sequence; 1 to 539 of the HMG1 sequence; 1 to 540 of the HMG1 sequence; 1 to 541 of the HMG1 sequence; 1 to 542 of the HMG1 sequence; 1 to 543 of the HMG1 sequence; 1 to 544 of the HMG1 sequence; 1 to 545 of the HMG1 sequence; 1 to 546 of the HMG1 sequence; 1 to 547 of the HMG1 sequence; 1 to 548 of the HMG1 sequence; 1 to 549 of the HMG1 sequence; 1 to 550 of the HMG1 sequence; 1 to 551 of the HMG1 sequence; and 1 to 552 of the HMG1 sequence. In one or more embodiments, additional methionine (M) may be added to a N-terminal of tHMG1. In one or more embodiments, additional alanine (A) may be added to a N-terminal of tHMG1.

In one or more embodiments, a truncated HMG2 (tHMG2) may include a portion of a N-terminal membrane anchor domain being removed. In one or more embodiments, removed domain may include amino acids from 1 to 520 of the HMG2 sequence; 1 to 521 of the HMG2 sequence; 1 to 522 of the HMG2 sequence; 1 to 523 of the HMG2 sequence; 1 to 524 of the HMG2 sequence; 1 to 525 of the HMG2 sequence; 1 to 526 of the HMG2 sequence; 1 to 527 of the HMG2 sequence; 1 to 528 of the HMG2 sequence; 1 to 529 of the HMG2 sequence; 1 to 530 of the HMG2 sequence; 1 to 531 of the HMG2 sequence; 1 to 532 of the HMG2 sequence; 1 to 533 of the HMG2 sequence; 1 to 534 of the HMG2 sequence; 1 to 535 of the HMG2 sequence; 1 to 536 of the HMG2 sequence; 1 to 537 of the HMG2 sequence; 1 to 538 of the HMG2 sequence; 1 to 539 of the HMG2 sequence; 1 to 540 of the HMG2 sequence; 1 to 541 of the HMG2 sequence; 1 to 542 of the HMG2 sequence; 1 to 543 of the HMG2 sequence; 1 to 544 of the HMG2 sequence; 1 to 545 of the HMG2 sequence; 1 to 546 of the HMG2 sequence; 1 to 547 of the HMG2 sequence; 1 to 548 of the HMG2 sequence; 1 to 549 of the HMG2 sequence; 1 to 550 of the HMG2 sequence; 1 to 551 of the HMG2 sequence; and 1 to 552 of the HMG2 sequence. In one or more embodiments, additional methionine (M) may be added to a N-terminal of tHMG2. In one or more embodiments, additional alanine (A) may be added to a N-terminal of tHMG2. In one or more embodiments, the genetically engineered microbe may have been modified such that the activity of the second enzyme is modulated by increasing an expression of the second nucleic acid sequence.

In one or more embodiments, the genetically engineered microbe may have been modified such that the activity of the second enzyme is modulated by increasing an expression of the second nucleic acid sequence.

The genetically engineered microbe further includes a heterologous nucleic acid sequence encoding a third enzyme, wherein the third enzyme is configured to catalyze an isoprene-producing chemical reaction. In one or more embodiments, the third enzyme may include at least a functional fragment of an isoprene synthase (IspS). In one or more embodiments, at least a functional fragment of an isoprene synthase (IspS) may include amino acid substitution F340L and A570N and is designated herein as “IspSm.” In one or more embodiments, a least a functional fragment of isoprene synthase (IspS) may include amino acid substitution S339C and G542S and further include the removal of 52 amino acids from the N-terminal portion of the functional fragment and is designated herein as “IspSf.” In one or more embodiments, the isoprene-producing chemical reaction may utilize DMAPP as a reactant.

In one or more embodiments, the genetically engineered microbe may have been modified such that the activity of a fourth enzyme is modulated by decreasing, attenuating, or deleting an expression of a fourth nucleic acid sequence encoding the fourth enzyme. In some cases, the fourth enzyme may include geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), pyruvate dehydrogenase (encoded by PDH) including pyruvate dehydrogenase E1 component (encoded by AceE), pyruvate oxidase (encoded by poxB), pyruvate decarboxylase (encoded by PDC), malate synthase (encoded by AceB or MLS1), citrate synthase (encoded by CIT1), ATP-citrate lyase (CitE) including AclY, AclB, and AclA, glycerol-producing enzymes including cGPDH and G3PP [41], ethanol-producing enzymes including invertase and zymase, alcohol dehydrogenase (encoded by ADH1), alcohol dehydrogenase enzyme (encoded by adhE), lactate-producing enzymes including lactate dehydrogenase such as lactate dehydrogenase A (encoded by IdhA) and D-lactate dehydrogenase (encoded by did), acetate-producing enzymes including acetyl coenzyme A (acetyl-CoA) hydrolases, and/or acetate kinase (encoded by ackA), among others.

In one or more embodiments, the genetically engineered microbe may include a fifth nucleic acid sequence encoding a fifth enzyme. In some cases, the fifth enzyme may include Alcohol Dehydrogenase 2 (ADH2), Triosephosphate Dehydrogenase 3 (TDH3), or Pyruvate Decarboxylase (PDC).

In one or more embodiments, genetically engineered microbe may be configured to produce an agent capable of inhibiting a phosphotransacetylase-acetate kinase (Pta-AckA) pathway, thereby facilitating an acetate reuptake. Agent may include Pta-AckA inhibitors such as tricostatin A and nicotinamide.

In one or more embodiments, genetically engineered microbe may include balanced methylerythritol phosphate (MEP) and MVA pathways.

The method of producing isoprene from ethanol includes culturing a genetically engineered microbe under suitable conditions. The genetically engineered microbe includes a first nucleic acid sequence encoding a first enzyme, wherein the first enzyme is configured to catalyze one or more steps of a conversion from ethanol to A-CoA, a second nucleic acid sequence encoding a second enzyme of the MVA pathway, and a heterologous nucleic acid sequence encoding a third enzyme, wherein the third enzyme is configured to catalyze an isoprene-producing chemical reaction. The method further includes providing a substrate to a cultured genetically engineered microbe to produce isoprene.

Aspects of the present disclosure may be used to provide a sustainable means of producing isoprene and similar value-added chemicals. Aspects of the present disclosure may be used to improve the carbon efficiency of biosynthetic techniques. Aspects of the present disclosure may be used to reduce the dependence on fossil fuels and mitigate the negative environmental impacts thereof. Exemplary embodiments illustrating aspects of the present disclosure are described below in the context of several specific examples.

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

It is understood that the acts described below are meant as a general overview and demonstration of an exemplary method, and that the method may include different and/or additional acts as described herein or otherwise.

While the present invention will be described as having particular configurations disclosed herein, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

It is to be understood that any aspect and/or element of any embodiment of the method(s) described herein or otherwise may be combined in any way to form additional embodiments of the method(s) all of which are within the scope of the method(s).

Where a process is described herein, those of ordinary skill in the art will appreciate that the process may operate without any user intervention. In another embodiment, the process includes some human intervention (for example, a step is performed by or with the assistance of a human).

For the purposes of this disclosure, including the claims, the phrase “at least some” means “one or more” and includes the case of only one. Thus, for example, the phrase “at least some ABCs” means “one or more ABCs” and includes the case of only one ABC.

For the purposes of this disclosure, including the claims, the term “at least one” should be understood as meaning “one or more” and therefore includes both embodiments that include one or multiple components. Furthermore, dependent claims that refer to independent claims that describe features with “at least one” have the same meaning, both when the feature is referred to as “the” and “the at least one”.

For the purposes of this disclosure, the term “portion” means some or all. Therefore, for example, “A portion of X” may include some of “X” or all of “X”. In the context of a conversation, the term “portion” means some or all of the conversation.

For the purposes of this disclosure, including the claims, the phrase “using” means “using at least” and is not exclusive. Thus, for example, the phrase “using X” means “using at least X”. Unless specifically stated by use of the word “only”, the phrase “using X” does not mean “using only X”.

For the purposes of this disclosure, including the claims, the phrase “based on” means “based in part on” or “based, at least in part, on” and is not exclusive. Thus, for example, the phrase “based on factor X” means “based in part on factor X” or “based, at least in part, on factor X”. Unless specifically stated by use of the word “only”, the phrase “based on X” does not mean “based only on X”.

In general, for the purposes of this disclosure, including the claims, unless the word “only” is specifically used in a phrase, it should not be read into that phrase.

For the purposes of this disclosure, including the claims, the phrase “distinct” means “at least partially distinct”. Unless specifically stated, distinct does not mean fully distinct. Thus, for example, the phrase “X is distinct from Y” means that “X is at least partially distinct from Y” and does not mean that “X is fully distinct from Y”. Thus, for the purposes of this disclosure, including the claims, the phrase “X is distinct from Y” means that X differs from Y in at least some way.

It should be appreciated that the words “first”, “second”, and so on, in the description and claims, are used to distinguish or identify, and not to show a serial or numerical limitation.

Similarly, letter labels (for example, “(A)”, “(B)”, “(C)”, and so on, or “(a)”, “(b)”, and so on) and/or numbers (for example, “(i)”, “(ii)”, and so on) are used to assist in readability and to help distinguish or identify, and are not intended to be otherwise limiting or to impose or imply any serial or numerical limitations or orderings. Similarly, words such as “particular”, “specific”, “certain”, and “given”, in the description and claims, if used, are to distinguish or identify, and are not intended to be otherwise limiting.

For the purposes of this disclosure, including the claims, the terms “multiple” and “plurality” mean “two or more,” and include the case of “two”. Thus, for example, the phrase “multiple ABCs” means “two or more ABCs” and includes “two ABCs”. Similarly, for example, the phrase “multiple PQRs” means “two or more PQRs” and includes “two PQRs”.

The present invention also covers the exact terms, features, values, and ranges, etc., in case these terms, features, values, and ranges, etc., are used in conjunction with terms such as “about”, “around”, “generally”, “substantially”, “essentially”, “at least”, etc. Thus, for example, “about 3” or “approximately 3” shall also cover exactly 3, and “substantially constant” shall also cover exactly constant.

For the purposes of this disclosure, unless stated otherwise, the terms “about” or “approximately” refer to a value that is within 10% above or below the value being described.

For the purposes of this disclosure, including the claims, singular forms of terms are to be construed as also including the plural form and vice versa, unless the context indicates otherwise. Thus, it should be noted that for the purposes of this disclosure, the singular forms “a”, “an”, and “the” include plural references unless the context clearly dictates otherwise. In other words, terms such as “a”, “an”, and “the” are not intended to refer to only a singular entity but include the general class of which a specific example may be used for illustration.

Throughout the description and claims, the terms “comprise”, “including”, “having”, “contain”, and their variations should be understood as meaning “including but not limited to” and are not intended to exclude other components unless specifically so stated.

For the purposes of this disclosure, the terms “administration” or “administering” refer to a method of giving a dosage of a compound or pharmaceutical composition to a subject.

For the purposes of this disclosure, the terms “treat”, “treating”, or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (for example, by reducing one or more symptoms of a neurological disorder). The term “therapeutic” includes the effect of mitigating deleterious clinical effects of certain processes (i.e., consequences of the process, rather than the symptoms of processes).

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and embodiments thereof are further described in the examples below, it is to be understood that the invention is not to be limited to the disclosed embodiment, but on the contrary, is intended to cover various modifications and equivalent arrangements included within the spirit and scope of the appended claims.

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, and/or components have not been described in detail in order to avoid unnecessarily obscuring claimed subject matter. The illustrative embodiments described in the detailed description and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

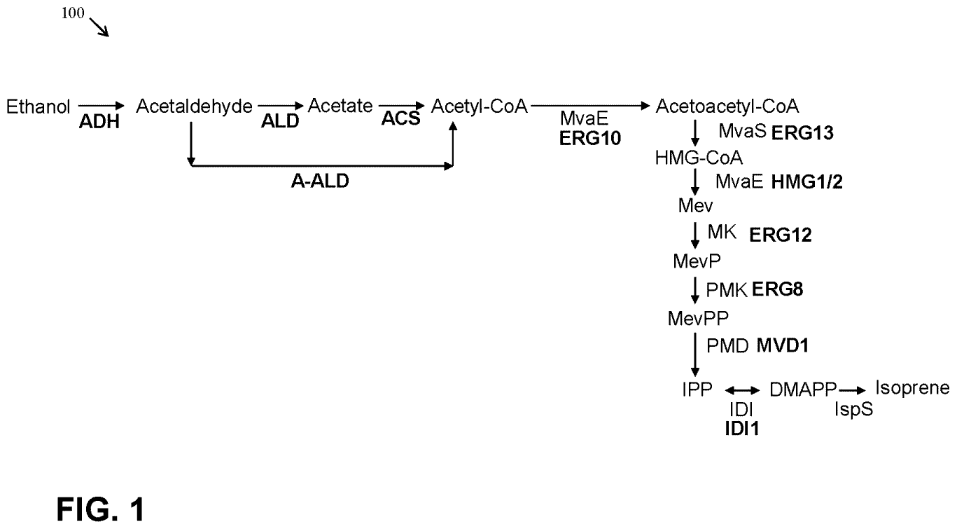

Referring now to FIG. 1, an exemplary embodiment of a metabolic pathway that converts ethanol to isoprene is illustrated as scheme 100. For the purposes of this disclosure, “isoprene”, also known as 2-methyl-1,3-butadiene, is a volatile organic compound with the chemical formula C₅H₈, a structural formula of CH₂═CHC(CH₃)═CH₂, and a normal boiling point of 34° C. Isoprene may be considered a “hemiterpene” (i.e., half of a terpene), and the details of terpenes will be described below in this disclosure. Isoprene is a key building block for synthesizing natural rubber and various other polymers. Additionally, isoprene may be used in the manufacture of synthetic rubber, adhesives, and elastomers, as well as in the production of pharmaceuticals and other specialty chemicals. Isoprene may be produced naturally by plants and trees, particularly in large amounts by the rubber tree (i.e., Hevea brasiliensis). It may also be produced industrially through the thermal cracking of petroleum-derived naphtha or, in some cases, through biotechnological methods using genetically engineered microbes, as described in this disclosure. In some cases, isoprene may be synthesized using industrial organic chemistry, via synthetic routes such as an acetone/acetylene route, a propylene dimer route, an isoamylene route, an isopentane route, or an isobutylene/formaldehyde route, among others (Whited et al, 2010).

With continued reference to FIG. 1, for the purposes of this disclosure, a “terpene” is an organic compound with the general molecular formula (C5H8)n, often characterized by a structure that contains repeating isoprene units. Terpenes are produced predominantly by plants, particularly conifers, and certain insects, and may be involved in various biological functions, including defense mechanisms and communication. Terpenes may be classified as monoterpenes (n=2), sesquiterpenes (n=3), diterpenes (n=4), and higher terpenes (n>4), based on the number of isoprene units they contain. Monoterpenes (C₁₀H₁₆) may include without limitation limonene found in citrus peels, myrcene in hops and lemongrass, pinene in pine resin, linalool in lavender, and geraniol in rose oil, among others. Sesquiterpenes (C₁₅H₂₄) may include without limitation farnesene in apple coatings, humulene in hops, bisabolol in chamomile, caryophyllene in black pepper, and nerolidol in ginger and tea tree oil. Diterpenes (C₂₀H₃₂) may include without limitation taxadiene from yew trees, gibberellins as plant hormones, phytol in chlorophyll, steviol in Stevia leaves, and retinol (Vitamin A1) derived from β-carotene. Higher terpenes may include without limitation squalene (C₃₀H₅₀) in shark liver oil, lanosterol (C₃₀H₅₀₀) in wool grease, tetraterpenes or tetraterpenoids including carotenoids (C₄₀H₅₆) such as beta-carotene in carrots, and rubber (polyterpene) from latex. Terpenes may serve as a biochemical precursor for a wide array of natural products, including essential oils, resins, and various pharmacologically active compounds, making them valuable in industries such as pharmaceuticals, perfumery, and agriculture. Isoprene belongs to a broader class of molecules called isoprenoids, and similarly, terpenes belong to a broader class of molecules called terpenoids, though it is worth noting that these terminologies may often be used interchangeably. An isoprenoid may include a molecule that is derived from isoprene, and similarly, a “terpenoid” may include a molecule that is derived from a terpene, consistent with details described above. Isoprenoids and terpenoids include a large and diverse class of naturally occurring organic compounds. Isoprenoids/terpenoids may also be synthesized through either the MEP pathway or the MVA pathway, as described in detail above. Isoprenoids/terpenoids may play essential roles in various biological processes, including without limitation cellular membrane structure (as cholesterol), photosynthesis (as carotenoids), and growth regulation (as gibberellins). Isoprenoids/terpenoids may also be used in pharmaceuticals, fragrances, and biofuels due to their diverse chemical properties and biological activities.

With continued reference to FIG. 1, it is worth noting that the invention described in this disclosure is not limited to production of isoprene only. As a nonlimiting example, the precursor for producing isoprene, such as without limitation IPP and/or DMAPP, may also be a building block for other isoprenoids, terpenes, or terpenoids; therefore, an accelerated biosynthesis of isoprene may contribute to an increase in yield for producing these isoprenoids, terpenes, or terpenoids. Specifically, in some cases, products of the MVA pathway such as DMAPP and IPP may be converted to geranyl diphosphate (GPP), which may be further converted to monoterpenes, etc. In some cases, products of the MVA pathway such as DMAPP and IPP may be converted to farnesyl diphosphate (FPP), which may be further converted to sesquiterpenes, triterpenes, and/or carotenoids, among others. A person of ordinary skill in the art, upon reviewing the entirety of this disclosure, will be able to recognize how the invention described herein may be extended to other related applications beyond isoprene synthesis.

With continued reference to FIG. 1, scheme 100 is implemented within a genetically modified microbe. For the purposes of this disclosure, a “genetically engineered microbe” is a microbe whose genetic material has been manipulated to alter one or more of its hereditary traits. Such manipulation may include without limitation inserting, deleting, or otherwise modifying one or more specific DNA sequences; as a result of such manipulation, a genetically modified microbe may exhibit one or more different traits in its structure, function, or the like, compared to its naturally occurring counterparts. For the purposes of this disclosure, a “microbe” is a microscopic organism, including bacteria, archaea, fungi, protozoa, viruses, and/or the like, that is capable of being utilized in one or more aspects of a biotechnological application. A microbe may be characterized by its ability to perform certain biological processes, such as without limitation fermentation, gene expression, and metabolite production, among others. A microbe may be harnessed for purposes such as without limitation recombinant protein production, bioremediation, synthesis of pharmaceuticals, and development of biofuels, among others, due to their diverse metabolic capabilities and ease of genetic manipulation.

With continued reference to FIG. 1, in one or more embodiments, genetically engineered microbe may include bacteria. With continued reference to FIG. 1, in one or more embodiments, genetically engineered microbe may include bacteria or fungi. In one or more embodiments, genetically engineered microbe may include Escherichia including Escherichia Coli (or E. coli), Vibrio including Vibrio natriegens (or V. natriegens), Saccharomyces including Saccharomyces cerevisiae (or S. cerevisiae), Aspergillus including Aspergillus niger. Aspergillus nidulans, and/or Aspergillus oryzae, Yarrowia including Yarrowia lipolytica (Y. lipolytica), Bacillus including Bacillus subtilis (B. subtilis), Clostridium including Clostridium ljungdahlii (C. ljungdahlii), and/or Pseudomonas including Pseudomonas putida (P. putida), and Ogataea including Ogataea parapolymorpha, Ogataea angusta, Ogataea thermomethanolica among others.

With continued reference to FIG. 1, for the purposes of this disclosure, Escherichia coli (E. coli) is a widely studied bacterium that serves as a crucial model organism in biotechnology and molecular biology. E. coli is a gram-negative, rod-shaped bacterium naturally found in the intestines of humans and other warm-blooded animals. The simplicity, well-characterized genetics, and rapid growth of E. coli, among other traits, make it a suitable host for genetic engineering applications. It is commonly used for producing recombinant proteins, plasmid cloning, and various metabolic engineering processes.

With continued reference to FIG. 1, for the purposes of this disclosure, Vibrio natriegens (V. natriegens) is a fast-growing, gram-negative marine bacterium. V. natriegens has recently gained prominence in biotechnology due to its exceptionally rapid doubling time of less than 10 minutes under suitable conditions. The fast growth, genetic tractability, high transformation efficiency, and ability to thrive in simple media of V. natriegens, among other traits, make it a suitable organism for various applications, including synthetic biology, recombinant protein production, metabolic engineering, and high-throughput screening, among others.

With continued reference to FIG. 1, for the purposes of this disclosure, Saccharomyces cerevisiae (S. cerevisiae) is a unicellular eukaryote, commonly known as brewer's yeast or baker's yeast, that plays a pivotal role in biotechnology due to its well-characterized genetics and ease of manipulation. S. cerevisiae has been instrumental in winemaking, baking, and brewing since ancient times and is a model system extensively used for recombinant DNA technology, metabolic engineering, and synthetic biology applications. Its ability to perform post-translational modifications similar to higher eukaryotes makes it suitable for producing complex proteins and biopharmaceuticals. Additionally, S. cerevisiae may be employed in biofuel production, fermentation processes, and various industrial biotechnology applications.

With continued reference to FIG. 1, for the purposes of this disclosure, Aspergillus is a genus of filamentous fungi. Aspergillus is characterized by its ability to produce a wide array of enzymes, organic acids, and secondary metabolites, making it a suitable host for production of biofuels, pharmaceuticals, and food products. Several genetic manipulation techniques have been established for Aspergillus. Certain Aspergillus species, such as Aspergillus niger, Aspergillus nidulans, and Aspergillus oryzae, may be used for recombinant protein production and metabolic engineering due to their efficient protein secretion systems and robust metabolic capabilities. Additionally, and/or alternatively, other filamentous fungi, such as without limitation, Fusarium and/or Neurospora, may be used.

With continued reference to FIG. 1, for the purposes of this disclosure, Yarrowia lipolytica (Y. lipolytica) is a species of yeast belonging to genus Yarrowia and the family Dipodascaceae. It is known for its ability to utilize a wide range of carbon sources, particularly lipids, which makes it a useful organism in biotechnological applications. Yarrowia lipolytica may be used in the production of various bioproducts, including single-cell oils, enzymes, and organic acids. Yarrowia lipolytica is also notable for its potential use in bioremediation, as it may degrade environmental pollutants such as hydrocarbons and fatty acids. Yarrowia lipolytica has a well-studied genetic system, allowing for genetic modifications and metabolic engineering to enhance its production capabilities. Additionally, Yarrowia lipolytica is recognized for its lack of toxicity in food and industrial applications, making it an attractive candidate for use in various biotechnological processes.

With continued reference to FIG. 1, for the purpose of this disclosure, Ogataea is a genus of ascomycetous yeasts in the family Saccharomycesaceae. It is known for its thermotolerance and metabolic versatility, which makes it valuable in industrial biotechnology applications including but not limited to production of recombinant proteins and bio-based chemicals. Ogataea parapolymorpha (O. parapolymorpha) is a thermotolerant methylotrophic yeast capable of utilizing methanol as its sole carbon and energy source and assimilate nitrate. (O. parapolymorpha has been extensively employed in the production of pharmaceuticals, and production of heterologous protein. Ogataea angusta (O. angusta) is a thermotolerant methylotrophic yeast capable of methylotrophy. It has been utilized in the production of recombinant proteins and vaccine development. Ogataea thermomethanolica (O. thermomethanolica) is a thermotolerant methylotrophic yeast that can be cultured in high cell densities using low-cost substrates like sucrose making it suitable for industrial protein production. It can be used for production of heterologous protein. Additionally, Ogataea species offer robust platforms for biotechnological applications due to their unique metabolic capabilities and adaptability to industrial processes.

With continued reference to FIG. 1, for the purposes of this disclosure, Bacillus subtilis (B. subtilis), is a Gram-positive, rod-shaped bacterium commonly found in soil and the gastrointestinal tracts of ruminants and humans. Bacillus subtilis is known for its ability to form endospores, which allow it to survive in harsh environmental conditions. Bacillus subtilis is widely studied for its role in various applications, including biotechnology, agriculture, and food production. Bacillus subtilis serves as a model organism for laboratory studies due to its genetic tractability and well-characterized physiology. Additionally, Bacillus subtilis may be utilized as a probiotic in animal feed and/or as a biocontrol agent in agriculture, promoting plant growth and/or protecting crops from pathogens. Its capability of producing enzymes, antibiotics, and/or other bioactive compounds further underscores its significance in various industrial processes.

With continued reference to FIG. 1, for the purposes of this disclosure, Clostridium ljungdahlii (C. ljungdahlii) is a gram-positive, anaerobic, rod-shaped bacterium that is part of the genus Clostridium. It is notable for its ability to perform autotrophic growth by utilizing a mixture of carbon monoxide (CO), carbon dioxide (CO₂), and hydrogen (H₂) (i.e., syngas) as sole carbon and energy sources, through a process known as the Wood-Ljungdahl pathway (acetyl-CoA pathway). Clostridium ljungdahlii is capable of converting syngas into value-added products, such as without limitation acetone, butanol, ethanol (ABE), and acetic acid, making it an important organism for industrial biotechnology applications, particularly in gas fermentation (for example, syngas fermentation) and biofuel production. Clostridium ljungdahlii holds promise for producing renewable fuels and chemicals from waste gases, such as those generated by industrial processes or gasified biomass. Specifically, an advantage of Clostridia as a host organism may derive from their ability to employ diverse carbon sources, including mono-, oligo- and polysaccharides that would be found in waste products, making the fermentation of industrial, agricultural and waste products conceivable.

With continued reference to FIG. 1, for the purposes of this disclosure, Pseudomonas putida (P. putida) is a gram-negative, rod-shaped, aerobic bacterium belonging to the genus Pseudomonas. Pseudomonas putida is commonly found in soil, water, and various environments where organic compounds are present. Pseudomonas putida is notable for its metabolic versatility, which allows it to degrade a wide range of organic compounds including without limitation hydrocarbons, aromatic compounds, and/or environmental pollutants such as without limitation toluene and naphthalene, making it an important organism for environmental bioremediation. Additionally, Pseudomonas putida may be used in various industrial and biotechnological applications due to its ability to produce valuable compounds and its resilience under harsh conditions. Pseudomonas putida may also be used to produce chemicals, such as biofuels, bioplastics, and other value-added compounds, through microbial fermentation and/or genetic engineering.

With continued reference to FIG. 1, in some cases, genetically engineered microbe may include acetic acid bacteria (AAB). For the purposes of this disclosure, an “acetic acid bacteria” is a type of Gram-negative bacteria capable of oxidizing ethanol to form acetic acid or acetate. AAB may include the family of Acetobacteraceae, which belongs to the order of Rhodospirillales. AAB are widespread in nature and play an important role in the production of food and beverages, such as without limitation vinegar and kombucha. AAB may also be used in the production of other metabolic products, such as without limitation gluconic acid, L-sorbose, and bacterial cellulose, among others, with potential applications in the food and biomedical industries. AAB may be categorized into at least 19 distinct genera and many different groups, species and subspecies. The classification and/or taxonomy of AAB has also undergone several modifications in the past based on morphological, physiological, and genetic characteristics of the AAB (Gomes et al, 2018). AAB may include Acetobacter such as without limitation Acetobacter aceti, Acetobacter cerevisiae, Acetobacter malorum, Acetobacter oeni, Acetobacter pasteurianus, Acetobacter pomorum, Acetobacter peroxydans, Acetobacter diazotrophicu, Acetobacter europaeus, Acetobacter hansenii, Acetobacter liquefaciens, Acetobacter xylinus, Acetobacter cibinongensis, Acetobacter estunensis, Acetobacter indonesiensis, Acetobacter nitrogenifigens, Acetobacter orientalis, Acetobacter orleanensis, Acetobacter tropicalis, Acetobacter lovaniensis, and Acetobacter syzygii. For the purposes of this disclosure, Acetobacter is a genus of Gram-negative, aerobic bacteria that belongs to the family Acetobacteraceae. These bacteria are characterized by their ability to convert ethanol to acetic acid in the presence of oxygen, a process utilized in the production of vinegar. Acetobacter species are known for their role in the fermentation of alcoholic beverages, as well as in the fermentation industry where they are used to produce vinegar and other acetic acid-based products. Acetobacter are ubiquitous in nature, commonly found in environments where ethanol is present, such as flowers, fruits, and alcoholic beverages. Acetobacter species are also noted for their role in the spoilage of wine and beer, where they can convert ethanol to acetic acid, leading to undesirable flavors and spoilage. Additionally, some species of Acetobacter have been studied for their potential applications in biotechnological processes, including the production of cellulose and other bio-based materials.

With continued reference to FIG. 1, AAB may include Gluconacetobacter such as without limitation Gluconacetobacter entanii, Gluconacetobacter liquefaciens, Gluconacetobacter azotocaptans, Gluconacetobacter diazotrophicus, Gluconacetobacter sacchari, Gluconacetobacter europaeus, Gluconacetobacter hansenii, Gluconacetobacter intermedius, Gluconacetobacter nataicola, Gluconacetobacter oboediens, Gluconacetobacter rhaeticus, Gluconacetobacter saccharivorans, Gluconacetobacter swingisii, and Gluconacetobacter xylinus. For the purposes of this disclosure, Gluconacetobacter is a genus of Gram-negative, rod-shaped, AAB within the family Acetobacteraceae. Similar to Acetobacter, Gluconacetobacter are also known for their ability to convert ethanol into acetic acid through oxidative fermentation. Unlike other acetic acid bacteria, some species within this genus, such as Gluconacetobacter xylinus, may be capable of synthesizing cellulose, making them important in various industrial applications. Such cellulose-producing capabilities of certain may be exploited in the production of bacterial cellulose, which may be used in various applications, including medical wound dressings, biodegradable materials, and as a component in composite materials. Gluconacetobacter may also participate in nitrogen fixation, contributing to plant growth by converting atmospheric nitrogen into a form that plants can utilize. Such features make Gluconacetobacter potentially valuable in agricultural biotechnology, particularly in developing biofertilizers.

With continued reference to FIG. 1, AAB may include Gluconobacter such as without limitation Gluconobacter oxydans, Gluconobacter albidus, Gluconobacter cerinus, and Gluconobacter frateurii. For the purposes of this disclosure, Gluconobacter is another genus of Gram-negative, rod-shaped bacteria within the family Acetobacteraceae. Acetobacteraceae are characterized by their ability to incompletely oxidize a wide variety of carbohydrates and alcohols to organic acids, primarily through the action of membrane-bound dehydrogenases. Unlike other acetic acid bacteria, in some cases, Gluconobacter species may not fully convert ethanol to acetic acid; instead, they may perform partial oxidation, making them useful in various industrial bioprocesses. Gluconobacter are also noted for their role in the biotechnological production of several important compounds, including dihydroxyacetone, gluconic acid, and ketogluconates. These compounds are valuable in the food, pharmaceutical, and chemical industries.

With continued reference to FIG. 1, AAB may include Komagataeibacter such as without limitation Komagataeibacter europaeus, Komagataeibacter hansenii, Komagataeibacter intermedius, Komagataeibacter medellinensis, Komagataeibacter oboediens, and Komagataeibacter xylinus. For the purposes of this disclosure, Komagataeibacter is another genus of Gram-negative, aerobic bacteria within the family Acetobacteraceae. Komagataeibacter species are rod-shaped and may have a high tolerance for acidic environments. Similar to Gluconacetobacter, in addition to production of acetic acid, Komagataeibacter are also known for their ability to produce bacterial cellulose, which may be used in various applications such as high-strength paper, medical wound dressings, composite materials, wound dressings, food products, and biomaterials, among others.

With continued reference to FIG. 1, additionally, and/or alternatively, AAB may include Acidomonas, Ameyamaea, Asaia, Bombella, Commensalibacter, Endobacter, Granulibacter, Kozakia, Neoasaia, Neokomagataea, Nguyenibacter, Saccharibacter, Swaminathania, Swingsia, and/or TanticharoeniAcetobacter, among others.

With continued reference to FIG. 1, in some cases, creating a genetically engineered microbe may involve using recombinant DNA technology, CRISPR-Cas9, gene cloning, and/or other molecular biology techniques to achieve desired phenotypic changes. For the purposes of this disclosure, “recombinant DNA technology” is a type of technology that involves a manipulation of DNA sequences to create new genetic combinations not found in nature. Recombinant DNA technology may include techniques such as gene cloning, insertion of DNA fragments from one organism into another, and/or use of vectors such as plasmids to transfer a genetic material. Recombinant DNA technology may be used to express new traits or produce biological products such as proteins, enzymes, and hormones and may play a pivotal role in fields such as genetic engineering, biotechnology, and pharmaceutical development.

With continued reference to FIG. 1, for the purposes of this disclosure, a “plasmid” is a circular, double-stranded DNA molecule distinct from a cell's chromosomal DNA and capable of autonomous replication. A plasmid may be used as a vector for insertion, expression, and propagation of foreign genes within a host organism. Such vectors may include specific sequences for an origin of replication, selectable markers, and cloning sites, enabling manipulation and study of genetic material for applications in research, biotechnology, and therapeutic development.

With continued reference to FIG. 1, for the purposes of this disclosure, “CRISPR-Cas9” is a genetic engineering tool, derived from a bacterial immune defense mechanism, that includes Clustered Regularly Interspaced Short Palindromic Repeats (CRISPR) sequences and the CRISPR-associated protein 9 (Cas9) enzyme and allows for precise, targeted modification of DNA within an organism. CRISPR-Cas9 may be programmed with a guide RNA to target a specific DNA sequence, enabling Cas9 enzyme to create double-strand breaks at one or more precise locations, thereby facilitating insertion, deletion, or modification of genetic material.

With continued reference to FIG. 1, in one or more embodiments, genetically engineered microbe may include a Crabtree-positive microbe. For the purposes of this disclosure, a “Crabtree-negative” microbe is a microbe that that does not exhibit the Crabtree effect. A Crabtree-negative microbe may maintain aerobic respiration and efficient ATP production via the tricarboxylic acid cycle and oxidative phosphorylation under high glucose conditions. Such characteristic makes Crabtree-negative microbes potentially valuable for biotechnological applications that require efficient biomass yield and metabolic productivity without the inhibitory effects of fermentation byproducts. Common Crabtree-negative microbes include without limitation E. coli, Pseudomonas aeruginosa (P. aeruginosa), Bacillus subtilis (B. subtilis), Corynebacterium glutamicum (C. glutamicum), and Saccharomyces kluyveri (S. kluyveri), among others. In contrast, a “Crabtree-positive” microbe is a microbe that exhibits the Crabtree effect. Common Crabtree-positive microbes include without limitation S. cerevisiae, Saccharomyces pastorianus (lager yeast), and Schizosaccharomyces pombe (fission yeast).

With continued reference to FIG. 1, for the purposes of this disclosure, the “Crabtree effect”, named after the English biochemist Herbert Grace Crabtree, is a phenomenon where high glucose concentrations inhibit cellular respiration, leading to fermentation even in the presence of oxygen. The Crabtree effect may offer a microbe certain evolutionary advantage, as the Crabtree effect may allow a microbe to quickly generate ATP and outcompete other microbes in glucose-rich environments, consistent with details described above. The production of ethanol may also inhibit the growth of competing microbes to provide an additional competitive edge.

With continued reference to FIG. 1, as a nonlimiting example, due to its Crabtree-positive nature, S. cerevisiae may outcompete other microbes by consuming sugar rapidly while producing ethanol via fermentation at a high rate. Additionally, unlike most other microbes for which ethanol is toxic, S. cerevisiae is tolerant of high ethanol concentrations. Furthermore, S. cerevisiae may utilize ethanol as its carbon source for growth in the absence of glucose, which may confer further advantage over other microbes. Due to the ability of S. cerevisiae to essentially reproduce and thrive under more stringent conditions, the use of S. cerevisiae for the invention described in this disclosure may offer advantages at a large scale due to its low requirement for more expensive hygienic practices. However, utilizing S. cerevisiae for production of other chemicals at high efficiencies may be challenging due to the need to remove ethanol production.

With continued reference to FIG. 1, in some cases, a genetically engineered microbe may include a diploid strain. For the purposes of this disclosure, a “diploid strain” is a type of organism or cell line that contains two complete sets of chromosomes, one from each parent. In genetic and metabolic engineering, diploid strains may be used to study gene function and regulation, create hybrid organisms, and/or enhance desirable traits. A diploid strain may provide a robust system for complementation tests, enabling researchers to determine the function of specific genes. Additionally, a diploid strain may exhibit increased genetic stability and adaptability, making them valuable for industrial applications such as fermentation and the production of biofuels and pharmaceuticals.

With continued reference to FIG. 1, a genetically engineered microbe includes a first nucleic acid sequence. For the purposes of this disclosure, a “nucleic acid sequence” is a sequence of nucleotides that contains genetic information encoding a peptide. “Nucleic acid sequence” and “gene” may be used interchangeably throughout this disclosure. A nucleic acid sequence may include a coding sequence that encodes a peptide and one or more regulatory elements that regulate the expression of the coding sequence. Exemplary embodiments of a regulatory element may include a promoter, an enhancer, a silencer, an insulator, an operator, and a response element, among others. For the purposes of this disclosure, a “promoter” is a DNA sequence where an RNA polymerase binds to initiate transcription. For the purposes of this disclosure, an “enhancer” is a distant element of DNA sequence that increases transcription rates by interacting with a promoter via DNA looping. For the purposes of this disclosure, a “silencer” is a DNA sequence that represses transcription when bound by specific proteins. For the purposes of this disclosure, an “insulator” is a DNA sequence that prevents the interaction between one or more enhancers and one or more promoters of neighboring genes. For the purposes of this disclosure, an “operator” is a DNA segment that regulates the transcription of adjacent genes. For the purposes of this disclosure, a response element is a DNA sequence that responds to external signals, allowing genes to be turned on or off in response to environmental changes. These regulatory elements may work together to ensure precise gene expression necessary for proper cellular function.

With continued reference to FIG. 1, in one or more embodiments, a nucleic acid sequence may include one or more operons. For the purposes of this disclosure, an “operon” is a functioning unit of DNA containing a cluster of genes under the control of a single promoter. It is commonly found in prokaryotes such as bacteria. These genes are transcribed together into a single messenger RNA strand and typically encode proteins that work together in a specific biological pathway. An operon may include a regulatory element, such as an operator as described above, where an activator or repressor protein may bind to increase or inhibit transcription. An operon may include one or more regulatory genes that encode one or more such activator or repressor proteins. A nonlimiting example of operon may include the lac operon, which regulates lactose metabolism in E. coli.

With continued reference to FIG. 1, the first nucleic acid sequence encodes a first enzyme. For the purposes of this disclosure, an “enzyme” is a biological catalyst, often a protein, having a three-dimensional structure specifically tailored for fitting a substrate or reactant and catalyzing a chemical reaction therefrom. Enzymes are often characterized by their high catalytic activity, high specificity toward substrates, and sensitivity to environmental factors such as temperature and pH. A substrate may interact with an enzyme (and its active/binding site) via a lock-and-key mechanism or an induced fit. For the purposes of this disclosure, a “catalyst” is a chemical capable of accelerating a chemical reaction by lowering at least an activation barrier along a reaction coordinate and increasing at least a rate constant associated with the at least an activation barrier. In some cases, to perform a catalytic function, a catalyst may first be consumed by one or more reactants to form one or more intermediates, then be regenerated as the one or more intermediates are converted to one or more products. In some cases, one or more reactants may bind to a catalyst, participate in a chemical reaction, then dissociate from the catalyst as one or more products. The catalytic function of a catalyst or enzyme may be described using mathematical tools such as Arrhenius equation, Eyring equation, Michaelis-Menten equation, Lineweaver-Burk equation, among others, as deemed suitable by a person of ordinary skill in the art upon reviewing the entirety of this disclosure.

With continued reference to FIG. 1, the first enzyme is configured to catalyze one or more steps of a conversion from ethanol to A-CoA. In one or more embodiments, the first enzyme may include ADH that converts ethanol to acetaldehyde. ADH may be encoded by a corresponding ADH gene or variations thereof. In one or more embodiments, the first enzyme may include ALD that converts acetaldehyde to acetate. ALD may be encoded by a corresponding ALD gene or variations thereof. In one or more embodiments, the first enzyme may include ACS or Acetate-CoA ligase that converts acetate to A-CoA. ACS may be encoded by a corresponding ACS gene or variations thereof. A-CoA may be further converted into isoprene, following the MVA pathway. Additionally, and/or alternatively, acylating acetaldehyde dehydrogenase (A-ALD), which is encoded by a corresponding A-ALD gene, may convert acetaldehyde to A-CoA, in a single step. Additional details will be provided below in this disclosure.

With continued reference to FIG. 1, in one or more embodiments, the genetically engineered microbe may have been modified such that the activity of first enzyme is modulated by increasing an expression of first nucleic acid sequence. In some cases, promoter engineering may be used to improve the transcriptional level of a nucleic acid sequence/gene. Promoter engineering modifies the promoter region to increase gene expression levels, thereby improving enzyme production and activity. In one or more embodiments, codon optimization may be used to improve the translational efficiency of a gene. For the purposes of this disclosure, “codon optimization” is a technique used in genetic engineering to improve the expression of a gene in a particular host organism. Codon optimization involves altering the DNA sequence of a gene to use codons that are more frequently preferred by the host organism's translational machinery. A codon optimization process may take into account a codon bias of the host, ensuring that a synthetic gene sequence is translated more efficiently into the desired protein. Codon optimization may enhance the yield and function of a protein, which may be crucial for various applications in biotechnology and synthetic biology. It is worth noting that promoter engineering and codon optimization are distinct yet complementary techniques in genetic engineering. Promoter engineering involves modifying the promoter region of a gene to enhance its expression by improving the binding efficiency of transcriptional machinery. Codon optimization, on the other hand, focuses on altering the coding sequence of a gene to use preferred codons of the host organism, thereby improving translation efficiency. While both aim to increase protein production, promoter engineering targets transcriptional levels, and codon optimization targets translational efficiency. Combining both techniques may in some cases synergistically enhance an overall gene expression.

With continued reference to FIG. 1, as a nonlimiting example, when genetically engineered microbe includes S. cerevisiae, in order to overcome its strong preference for ethanol production, S. cerevisiae may be engineered such that the ethanol naturally produced by S. cerevisiae may serve as a carbon source for production of isoprene. Since production of isoprene may utilize A-CoA as a substrate, an increase in cytosolic acetyl-CoA production from ethanol may result in an increase in the yield of isoprene.

With continued reference to FIG. 1, genetically engineered microbe further includes a second nucleic acid sequence encoding a second enzyme of the mevalonate (MVA) pathway. In one or more embodiments, the second enzyme may include acetoacetyl-coenzyme A thiolase or 3-hydroxy-3-methylglutaryl-coenzyme A reductase that converts A-CoA to acetoacetyl-coenzyme A (AA-CoA), which can be encoded by the MvaE gene or ERG10 gene or variations thereof. In one or more embodiments, the second enzyme may include HMG-COA synthase that converts AA-CoA to HMG-COA and may be encoded by the MvaS gene or the ERG13 gene or variations thereof. In one or more embodiments, the second enzyme may include HMG-COA reductase that reduces HMG-COA to form Mev and may be encoded by the MvaE gene, the HMG1 gene, or HMG2 gene, or variations thereof. In one or more embodiments, the second enzyme may include mevalonate kinase (MK) that phosphorylates Mev to form Mev-P or MevP. MK may be encoded by ERG12 gene or variations thereof. In one or more embodiments, the second enzyme may include phosphomevalonate kinase (PMK) that phosphorylates Mev-P to form Mev-PP or MevPP. PMK may be encoded by ERG8 gene or variations thereof. In one or more embodiments, the second enzyme may include mevalonate-5-diphosphate decarboxylase or diphosphomevalonate decarboxylase to decarboxylates Mev-PP to produce IPP and may be encoded by MVD1 gene or variations thereof. In one or more embodiments, the second enzyme may include IPP-ispomerase that isomerizes IPP to form DMAPP and may be encoded by the IDI or the IDI1 gene or variations thereof.

With continued reference to FIG. 1, in one or more embodiments, genetically engineered microbe may have been modified such that the activity of second enzyme is modulated by increasing an expression of second nucleic acid sequence, consistent with details described above pertaining to first enzyme and/or first nucleic acid sequence.

With continued reference to FIG. 1, genetically engineered microbe further includes a heterologous nucleic acid sequence encoding a third enzyme. For the purposes of this disclosure, a “heterologous” nucleic acid sequence is a nucleic acid sequence, as described above in this disclosure, that originates from a different species or is artificially synthesized and introduced into a host organism. A heterologous nucleic acid sequence may be inserted into a host genome or expressed in a host cell to produce proteins, confer new traits, and/or study gene functions. A heterologous nucleic acid may include genes, regulatory elements, or other sequences that are not naturally found in the host organism's genome.

With continued reference to FIG. 1, the third enzyme is configured to catalyze an isoprene-producing chemical reaction. For the purposes of this disclosure, an “isoprene-producing reaction” is a chemical reaction that forms one or more products that include isoprene. In one or more embodiments, the third enzyme may include at least a functional fragment of IspS. For the purposes of this disclosure, a “functional fragment” of an enzyme is a part of the enzyme that includes at least a catalytically active site and therefore preserves at least a catalytic function of the enzyme. In some cases, such functional fragments may be created by truncating or simplifying an enzyme to eliminate peripheral, nonessential structures to improve one or more aspects of its properties, such as without limitation solubility. IspS may be derived from plants, as many plants such as kudzu (the Asian vine, Pueraria montana), aspen (Populus tremuloides), and poplar (Populus), among others, may naturally produce isoprene by the catalytic elimination of pyrophosphate from DMAPP.) In one or more embodiments, at least a functional fragment of an isoprene synthase (IspS) may include amino acid substitution F340L and A570N and is designated herein as “IspSm.” In one or more embodiments, a least a functional fragment of isoprene synthase (IspS) may include amino acid substitution S339C and G542S, and further include the removal of 52 amino acids from the N-terminal portion of the functional fragment and is designated herein as “IspSf.” In one or more embodiments, isoprene-producing chemical reaction may utilize DMAPP as a reactant.

With continued reference to FIG. 1, in one or more embodiments, genetically engineered microbe may have been modified such that the activity of a fourth enzyme is modulated by decreasing, attenuating, downregulating, or deleting an expression of a fourth nucleic acid sequence encoding the fourth enzyme. The fourth enzyme may include any enzyme that may catalyze competing side reaction and/or contribute to a reduced yield of isoprene, as recognized by a person of ordinary skill in the art upon reviewing the entirety of this disclosure. Additional details will be provided below in this disclosure. In some cases, the fourth enzyme may include geranyl diphosphate synthase (IspA), farnesyl pyrophosphate synthetase (Erg 20), squalene synthase (ERG9), geranyl diphosphate synthase (GPPS), geranylgeranyl diphosphate synthase (BTS1), farnesyl diphosphate synthase (FPPS), pyruvate dehydrogenase (encoded by PDH) including pyruvate dehydrogenase E1 component (encoded by AceE), pyruvate oxidase (encoded by poxB), pyruvate decarboxylase (encoded by PDC), malate synthase (encoded by AceB or MLS1), citrate synthase (encoded by CIT1), ATP-citrate lyase (CitE) including AclY, Ac1B, and Ac1A, glycerol-producing enzymes including cGPDH and G3PP [41], ethanol-producing enzymes including invertase and zymase, alcohol dehydrogenase (encoded by ADH1), alcohol dehydrogenase enzyme (encoded by adhE), lactate-producing enzymes including lactate dehydrogenase such as lactate dehydrogenase A (encoded by IdhA) and D-lactate dehydrogenase (encoded by did), acetate-producing enzymes including acetyl coenzyme A (acetyl-CoA) hydrolases, acetate kinase (encoded by ackA), phosphatase, and/or hydrolase (for example, a hydrolase encoded by the NudB gene), Peroxisomal membrane E3 ubiquitin ligase (PEX10) (encoded by PEX10 gene), among others. In some cases, a gene encoding an enzyme may be knocked down or knocked out. In some cases, disrupting expression of a fourth nucleic acid sequence encoding the fourth enzyme may include deleting an upstream activation sequence in a promoter of squalene synthase (ERG9), or the nucleic acid sequence encoding peroxisomal membrane E3 ubiquitin ligase (PEX10). As a nonlimiting example, when unable to produce ethanol due to a genetic modification, such as a removal of PDC gene encoding PDC, an enzyme that catalyzes the conversion from pyruvate to acetaldehyde, S. cerevisiae may become glucose-sensitive and may only grow on carbon sources containing two carbon atoms (for example, ethanol or acetate) without glucose present; this may improve the carbon efficiency of one or more metabolic pathways described in this disclosure, resulting a higher yield of isoprene production.

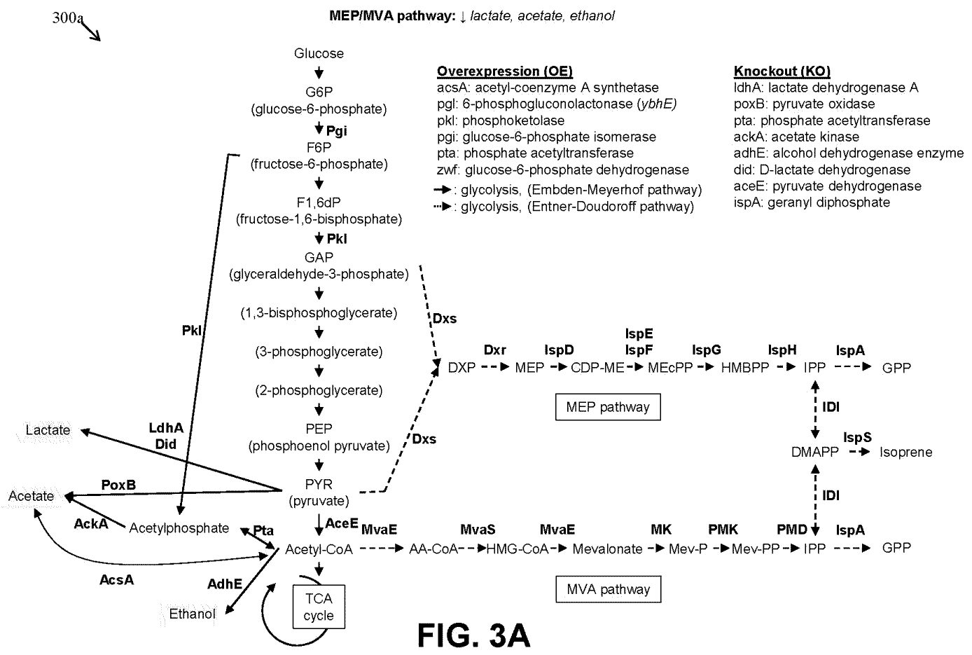

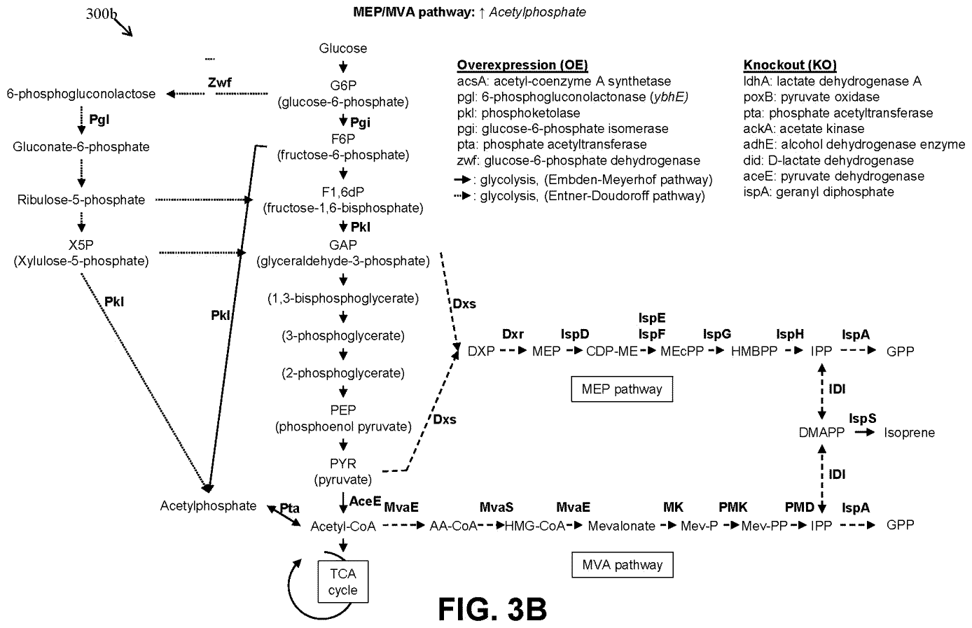

With continued reference to FIG. 1, in one or more embodiments, genetically engineered microbe may be configured to produce an agent capable of inhibiting a Pta-AckA pathway. The Pta-AckA pathway is a key metabolic route in many bacteria and may be particularly significant under anaerobic conditions or when there is an excess of carbon sources, as it allows a cell to dispose of excess A-CoA and generate additional ATP. Inhibition of the Pta-AckA pathway may facilitate an acetate reuptake and improve the overall carbon efficiency of isoprene production. The Pta-AckA pathway involves two main enzymes: Pta catalyzes the conversion of A-CoA to acetyl phosphate, whereas AckA catalyzes the conversion of acetyl phosphate to acetate to generate ATP. By inhibiting these enzymes, the production of acetate may be reduced. Such reduction may be beneficial in preventing acetate accumulation that can be detrimental to cellular growth and productivity. Additional details will be provided below in FIGS. 3A-B.

With continued reference to FIG. 1, a genetically engineered microbe may be configured to produce a Pta-AckA inhibitor. For the purposes of this disclosure, a “phosphotransacetylase-acetate kinase inhibitor” is a chemical compound that interferes with the activity of the phosphotransacetylase (Pta) and/or acetate kinase (AckA). Nonlimiting examples of Pta-AckA inhibitors may include deacetylase inhibitors such as tricostatin A and nicotinamide. Specifically, such inhibitors may function by regulating acetyl phosphate-dependent protein acetylation.

With continued reference to FIG. 1, in one or more embodiments, genetically engineered microbe may include balanced MEP and MVA pathways. Additional details pertaining to the MEP pathway will be provided below in this disclosure. Specifically, leveraging the MEP and MVA pathways may enhance the yield and production efficiency of isoprene by optimizing the flux of metabolites, providing metabolic redundancy, and improving the robustness of the production strain under varying conditions. In some cases, such balanced pathways may be implemented using a shunt. Such balanced pathways may offer several benefits. As a nonlimiting example, such balanced pathways may lead to a more efficient utilization of substrates, thereby reducing waste and improving the carbon economy of a genetically engineered microbe. Additionally, as a further nonlimiting example, such balanced pathways may allow for finer control over metabolic fluxes through genetic and regulatory modifications, facilitating the tuning of pathway activities to match specific production needs. Overall, such balanced pathways may enhance the efficiency, robustness, and flexibility of isoprene production, making it more economically viable and sustainable for industrial applications. As another nonlimiting example, the MEP and the MVA pathways may be balanced by increasing a flux through the MEP pathway to reduce carbon loss to the TCA cycle; this strategy may lead to an increase in isoprene production. A theoretical calculation of isoprene yield based upon flux ratios through the MEP and MVA pathways suggests that isoprene yield may increase with an increasing flux along the MEP pathway, with a maximum yield of approximately 0.31 g of isoprene per gram of glucose when the flux ratio is approximately 60:40.

With continued reference to FIG. 1, in one or more embodiments, genetically engineered microbe may include a fifth nucleic acid sequence encoding a fifth enzyme. The fifth enzyme may be encoded by a nucleic acid sequence that additionally functions as a promoter or regulatory element, such that its incorporation into a genetic construct promotes or enhances transcription of at least a downstream coding sequence. A “promoter,” for the purpose of this disclosure, is a specific DNA sequence located upstream of a gene that serves as the binding site for RNA polymerase and other transcription factors. In one or more embodiments, a fifth enzyme is capable of driving expression of certain proteins under certain fermentation and metabolic conditions. In one or more embodiments, a fifth enzyme may facilitate the conversion of a carbon source into key precursor metabolites required for initiating the mevalonate pathway. For example, without limitation, the fifth enzyme may include pyruvate decarboxylase (PDC), which catalyzes the decarboxylation of pyruvate to form acetaldehyde. In other embodiments, the fifth enzyme may include alcohol dehydrogenase 2 (ADH2), which oxidizes ethanol to acetaldehyde. The produced acetaldehyde may subsequently be converted to acetyl-CoA, a key precursor molecule required for initiating the mevalonate pathway (MVA pathway) for isoprene biosynthesis. In one or more embodiments, the inclusion of fifth enzyme may enable more efficient routing of carbon flux towards the biosynthesis of acetyl-CoA, thereby enhancing the flux into the MVA pathway. Additional details would be apparent to a person of ordinary skill in the art upon reviewing the entirety of this disclosure.

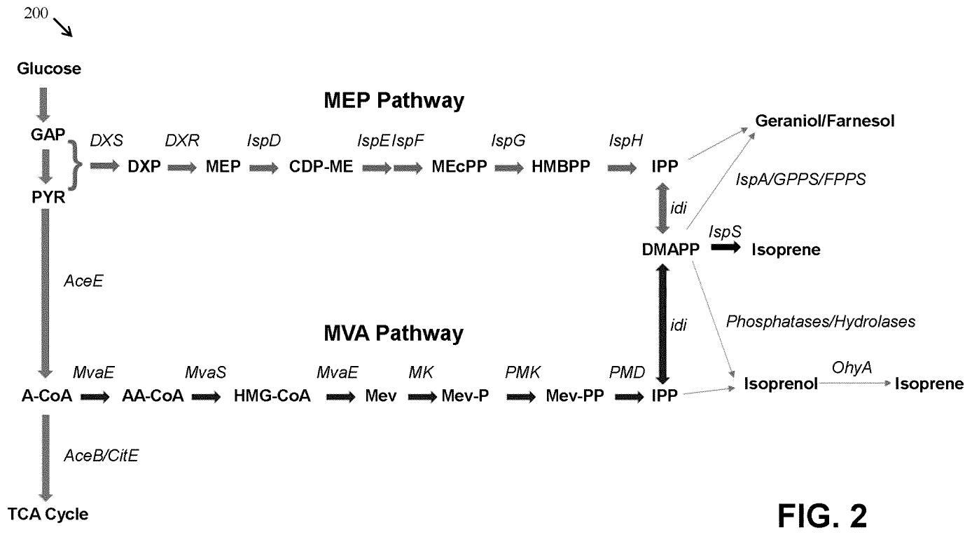

Referring now to FIG. 2, scheme 200 illustrates several metabolic pathways pertaining to biosynthesis of isoprene, highlighting the substrate, product, and/or enzyme (catalyst) associated with each step. Scheme 200 includes part of the central metabolic pathway, i.e., the cellular respiration pathway, wherein glucose is broken down to feed the tricarboxylic acid (TCA) cycle. Conversion of glucose involves several key steps. Initially, glucose (C₆H₁₂O₆) undergoes glycolysis by converting to two molecules of glyceraldehyde 3-phosphate (GAP). Such conversion is catalyzed by enzymes including hexokinase, phosphofructokinase, and aldolase. GAP is then further processed to form pyruvate (C₃H₃O₃⁻), producing a net gain of 2 ATP and 1 NADH. Such conversion is catalyzed by enzymes such as glyceraldehyde 3-phosphate dehydrogenase and pyruvate kinase. Pyruvate is further converted to acetyl coenzyme A (A-CoA), releasing NADH and CO₂. Such conversion is catalyzed by pyruvate dehydrogenase (PDH), which may include pyruvate dehydrogenase E1 component (encoded by gene AceE). A-CoA then enters the TCA cycle (not shown) and participates in reactions catalyzed by enzymes such as without limitation malate synthase (AceB) or ATP citrate lyase (CitE). It is worth noting that glucose is not the only viable carbon source for the invention described in this disclosure; alternatives such as sucrose, biomass, glycerol, ethanol, plant oils, or the like, may be used instead upon strategic engineering of metabolic pathways.

With continued reference to FIG. 2, scheme 200 includes methylerythritol phosphate (MEP) and mevalonate (MVA) pathways for isoprene production. Both the MEP and the MVA pathways are connected to the central metabolic pathway and may lead to production of isoprene, consistent with details described above. For the purposes of this disclosure, the methylerythritol phosphate (MEP) pathway, also known as the non-mevalonate pathway, is a metabolic route for isoprenoid biosynthesis in bacteria, algae, and/or plant plastids. It begins with the formation of 1-deoxy-D-xylulose-5-phosphate (DXP) from pyruvate and glyceraldehyde-3-phosphate (GAP), which is catalyzed by 1-deoxy-D-xylulose-5-phosphate synthase (DXS). This step is one of the rate-determining steps of the MEP pathway. DXP is then converted to 2-C-methyl-D-erythritol-4-phosphate (MEP), which is catalyzed by 1-deoxy-D-xylulose 5-phosphate reductase (DXR). MEP is then converted to 4-diphosphocytidyl-2-C-methyl-D-erythritol (CDP-ME), which is catalyzed by 4-diphosphocytidyl-2-C-methyl-D-erythritol synthase (IspD). CDP-ME is further converted via two steps to 2-C-methyl-D-erythritol-2,4-cyclodiphosphate (MEcPP), which is catalyzed by 4-diphosphocytidyl-2-C-methyl-Derythritol kinase (IspE) and 2-C-methyl-D-erythritol 2,4-cyclodiphosphate synthase (IspF). MEcPP is then reduced to 4-hydroxy-3-methylbut-2-enyl diphosphate (HMBPP), which is catalyzed by (E)-4-Hydroxy-3-methylbut-2-enyl pyrophosphate synthase (IspG). HMBPP is then converted to isopentenyl diphosphate (IPP) and dimethylallyl diphosphate (DMAPP), which is catalyzed by (E)-4-Hydroxy-3-methylbut-2-enyl pyrophosphate reductase (IspH or HMBPP reductase). IPP and DMAPP may establish a chemical equilibrium and interconvert with one another, which is catalyzed by isopentenyl diphosphate isomerase (idi, SEQ ID NO: 18). DMAPP may be converted to isoprene, which is catalyzed by isoprene synthase (IspS). IspS may be derived from plants, as many plants such as kudzu (the Asian vine, Pueraria montana), aspen (Populus tremuloides), and poplar (Populus), among others, may naturally produce isoprene by the catalytic elimination of pyrophosphate from DMAPP. It is worth noting that, in order to utilize ethanol as a carbon source and the MVA pathway, the MEP pathway may sometimes be suppressed, at least to some extent, in order to direct the flux towards the MVA pathway. Chemical structures of each reactant, intermediate, and product of the MEP pathway are illustrated in Scheme 1 below:

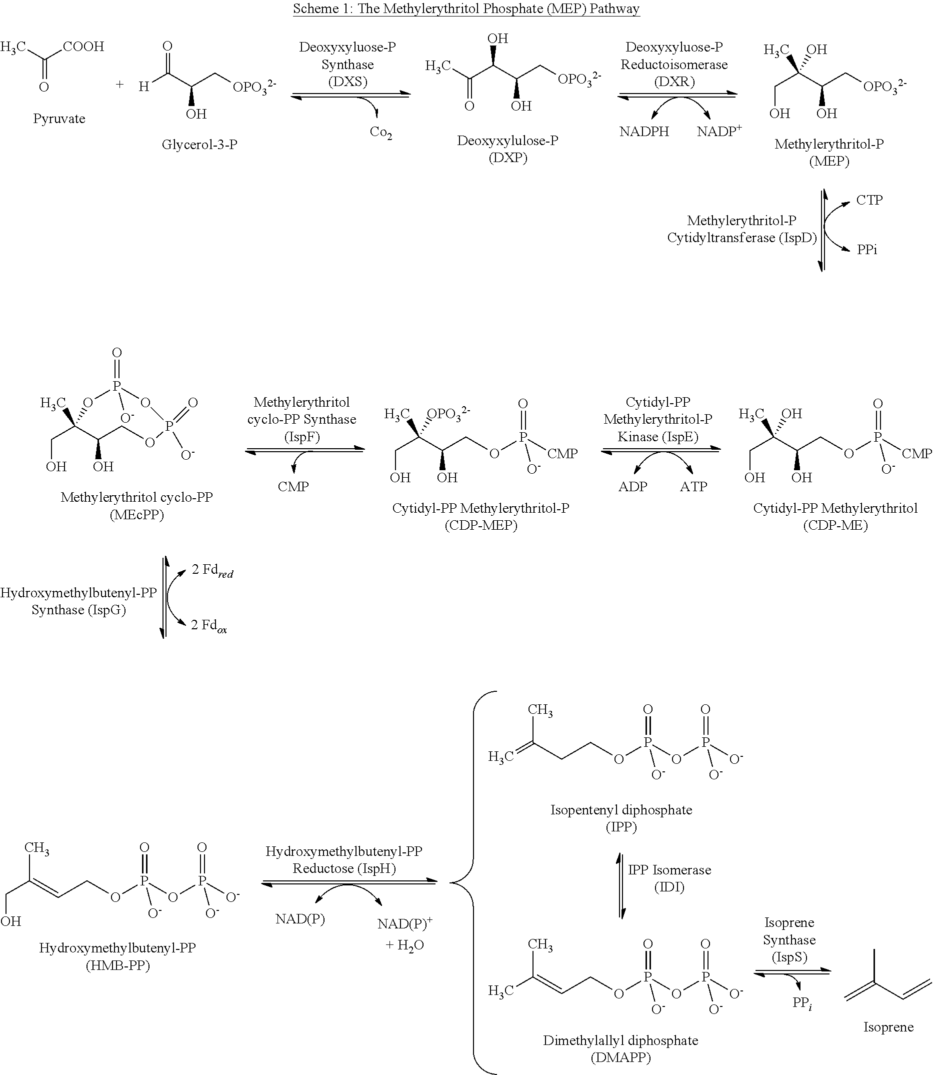

With continued reference to FIG. 2, for the purposes of this disclosure, the mevalonate (MVA) pathway is another metabolic route for isoprenoid biosynthesis, found in archaea, fungi, animals, and some bacteria. The MVA pathway starts with the conversion of A-CoA to acetoacetyl-coenzyme A (AA-CoA), which is catalyzed by acetoacetyl-coenzyme A thiolase or 3-hydroxy-3-methylglutaryl-coenzyme A reductase (MvaE, SEQ ID NO: 4, which may be encoded by gene ERG10, SEQ ID NO: 3). AA-CoA is then converted to 3-hydroxy-3-methylglutaryl-CoA (HMG-COA), which is catalyzed by HMG-COA synthase (MvaS, SEQ ID NO: 8, which may be encoded by gene ERG13, SEQ ID NO: 7). HMG-COA is then reduced to mevalonate (Mev). This step is catalyzed by MvaE, SEQ ID NO: 10, 12, which may be encoded by gene HMG1 (SEQ ID NO: 9), HMG2 (SEQ ID NO: 11), or HMG-COA reductase (HMGR). This step is the rate-determining step of the MVA pathway. Mev is phosphorylated to mevalonate-5-phosphate (Mev-P), which is catalyzed by mevalonate kinase (MK, SEQ ID NO: 6), which could be encoded by ERG12 (SEQ ID NO: 5). Mev-P is phosphorylated to form mevalonate-5-diphosphate (Mev-PP), which is catalyzed by phosphomevalonate kinase (PMK, SEQ ID NO: 2), which could be encoded by ERG8 (SEQ ID NO: 1). Mev-PP is subsequently decarboxylated to produce IPP, which is catalyzed by mevalonate-5-diphosphate decarboxylase or diphosphomevalonate decarboxylase (PMD, SEQ ID NO: 16), which may be encoded by MVD1 (SEQ ID NO: 15). IPP and DMAPP may establish a chemical equilibrium and interconvert with one another, which is catalyzed by idi (SEQ ID NO: 18), consistent with details described above. The idi enzyme may be encoded by IDI1 (SEQ ID NO: 17). DMAPP may be converted to isoprene, which is catalyzed by IspS, consistent with details described above. In some cases, the MVA pathway may be further categorized into the “upper pathway”, which converts A-CoA to MVA, and the “lower pathway”, which produces DMAPP from MVA. In some cases, the MVA pathway may be introduced (i.e., cloned) into bacteria such as E. coli (which only has a native MEP pathway) as two synthetic operons, using a combination of bacterial and yeast enzymes. As a nonlimiting example, the lower pathway operon may be integrated into the chromosome while the upper pathway operon may be expressed on one or more plasmids, optionally alongside an IspS-encoding gene or the like. Chemical structures of each reactant, intermediate, and product of the MVP pathway are illustrated in Scheme 2 below:

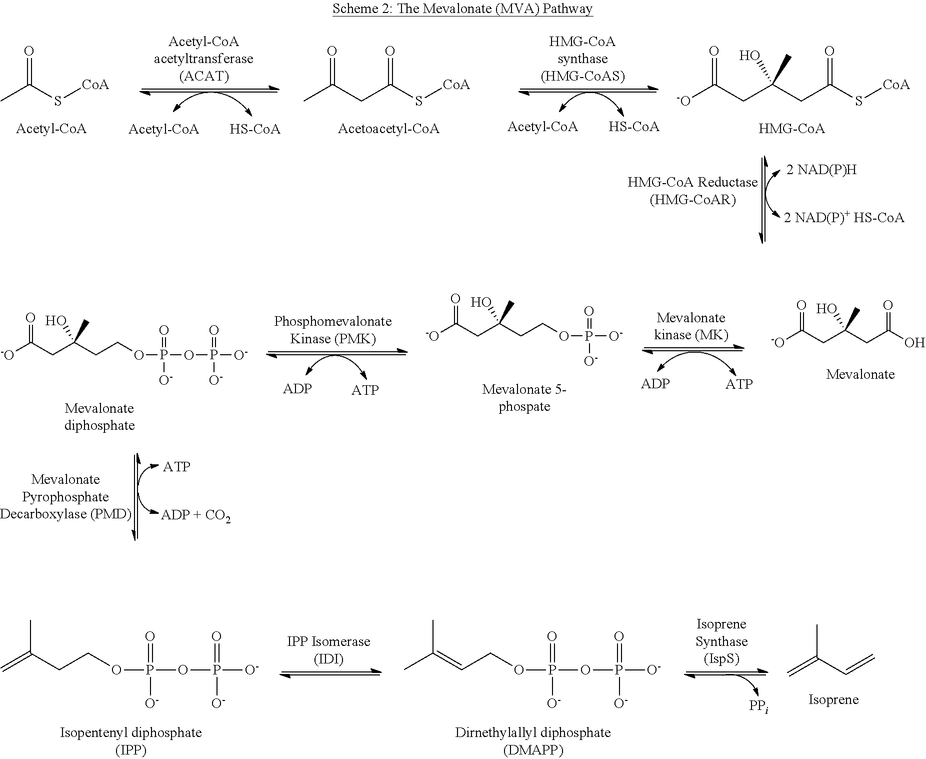

With continued reference to FIG. 2, scheme 200 includes a plurality of competing pathways that may limit the yield of isoprene production. IPP and/or DMAPP may be converted to geraniol and/or farnesol, which is catalyzed by enzymes such as without limitation geranyl diphosphate synthase (IspA), geranyl diphosphate synthase (GPPS), and/or farnesyl diphosphate synthase (FPPS). IPP and/or DMAPP may be converted to isoprenol, which is catalyzed by enzymes such as phosphatases and/or hydrolases including the Nudix hydrolase encoded by the NudB gene. As part of the central metabolic pathway, A-CoA may be converted to citrate to enter the tricarboxylic acid cycle, which is catalyzed by enzymes such as ATP citrate lyase (CitE) including AclY, Ac1B, and/or Ac1A. These pathways compete with the formation of isoprene. In some cases, to improve the yield of isoprene, byproduct isoprenol may be converted back to isoprene, which may be catalyzed by enzymes such as 3-methyl-3-buten-1-ol dehydroxylase (OhyA) or the like.

Referring now to FIGS. 3A-B, exemplary embodiments 300a-b of expanded metabolic pathways pertaining to isoprene biosynthesis are illustrated. FIGS. 3A-B include a plurality of potential gene targets. These gene targets and the activity thereof may be upregulated (for example, overexpressed), downregulated (for example, knocked down (KD)/knocked out (KO)), or otherwise modified, either individually or in combination, to increase the yield of isoprene.

With continued reference to FIGS. 3A-B, in one or more embodiments, the inherent activity of an enzyme may be improved by modifying the nucleic acid sequence that encodes the enzyme. Various techniques may be used to improve the inherent activity of an enzyme. As a nonlimiting example, directed evolution may be used to mimic natural selection in the lab by creating a large library of enzyme variants and selecting those with improved traits. As another nonlimiting example, site-directed mutagenesis may be used to introduce one or more specific mutations to enhance enzyme activity or stability. As another nonlimiting example, gene shuffling may be used to recombine segments of related genes to create new variants with enhanced properties. As another nonlimiting example, fusion proteins may be created by combining enzymes with other proteins to improve their function and/or stability. As a nonlimiting example, an enzyme such as OhyA or the like may be engineered to increase its inherent activity, thereby facilitating accumulation of isoprene while lifting the potentially toxic impact of isoprenol. As another nonlimiting example, an enzyme such MvaE or HMGR may be truncated from its naturally existing counterpart to improve its catalytic activity. As another nonlimiting example, one or more cofactors for one or more enzymes may be switched in order to improve the catalytic activity of an enzyme or enable fermentation conditions to be utilized that are more cost effective (e.g., with a lower oxygen requirement).

With continued reference to FIGS. 3A-B, in one or more embodiments, genes encoding enzymes such as 1-deoxy-D-xylulose-5-phosphate synthase (DXS), isoprene synthase (IspS), 3-hydroxy-3-methylglutaryl-coenzyme A reductase (HMG-COA reductase or HMGR), 3-methyl-3-buten-1-ol dehydroxylase (OhyA) or OhyA-like enzymes, (E)-4-hydroxy-3-methylbut-2-enyl diphosphate synthase (IspG), 1-deoxy-D-xylulose 5-phosphate reductase (DXR), (E)-4-hydroxy-3-methylbut-2-enyl diphosphate reductase (IspH or HMBPP reductase), acetyl-CoA C-acetyltransferase/acetoacetyl-coenzyme A thiolase (MvaE, encoded by gene ERG10), HMG-CoA Synthase (MvaS, encoded by gene ERG13), acylating acetaldehyde dehydrogenase (A-ALD), among others, may be modified to increase the inherent activity of the enzyme, consistent with details described in the rest of this disclosure.

With continued reference to FIGS. 3A-B, in one or more embodiments, the activity of an enzyme may be modulated by increasing or upregulating an expression of a nucleic acid construct that encodes the enzyme. In some cases, a coding sequence may be configured to upregulate one or more genes encoding enzymes such as phosphatase, hydrolase, 3-methyl-3-buten-1-ol dehydroxylase (OhyA), transcription factor (encoded by UPC2), acetyl-CoA C-acetyltransferase/acetoacetyl-coenzyme A thiolase (MvaE, encoded by gene ERG10), mevalonate kinase (MK, encoded by ERG12), HMG-COA synthase (MvaS, encoded by gene ERG13), pantothenate kinase (encoded by CAB1), dihydrolipoamide acetyltransferase component of the pyruvate dehydrogenase complex (encoded by LAT1), glucose-6-phosphate dehydrogenase (encoded by zwf), 6-phosphogluconolactonase (ybhE, encoded by pgl), phosphoketolase (encoded by pkl), glucose-6-phosphate isomerase (encoded by pgi), and phosphate acetyltransferase (encoded by pta), among others, as these genes encode enzymes that may contribute to the production and/or accumulation of isoprene. In some cases, regulation of a single gene may result in a regulation of multiple enzymes. As a nonlimiting example, when UPC2 is upregulated, ERG12 and ERG13 may also be overexpressed.

With continued reference to FIG. 3A-B, in one or more embodiments, in order to utilize ethanol as a carbon source for production of isoprene, the activity of an enzyme may be modulated by decreasing, attenuating, downregulating, or deleting the expression of a nucleic acid construct that encodes the enzyme. In some cases, a gene encoding an enzyme may be knocked down or knocked out. As nonlimiting examples, one or more genes encoding enzymes such as geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), pyruvate dehydrogenase (encoded by PDH) including pyruvate dehydrogenase E1 component (encoded by AceE), pyruvate oxidase (encoded by poxB), pyruvate decarboxylase (encoded by PDC), malate synthase (encoded by AceB or MLS1), citrate synthase (encoded by CIT1), ATP-citrate lyase (CitE) including AclY, Ac1B, and Ac1A, glycerol-producing enzymes including cGPDH and G3PP [41], ethanol-producing enzymes including invertase and zymase, alcohol dehydrogenase (encoded by ADH1), alcohol dehydrogenase enzyme (encoded by adhE), lactate-producing enzymes including lactate dehydrogenase such as lactate dehydrogenase A (encoded by IdhA) and D-lactate dehydrogenase (encoded by did), acetate-producing enzymes including acetyl coenzyme A (acetyl-CoA) hydrolases, acetate kinase (encoded by ackA), phosphatase, and hydrolase (for example, a hydrolase encoded by the NudB gene), among others, may be downregulated. However, it is worth noting that, in some cases, an enzyme may perform an essential function of a chassis and should not be completely knocked out or suppressed. As a nonlimiting example, farnesyl pyrophosphate synthetase encoded by ERG20 is essential to S. cerevisiae, and accordingly, specific sites within ERG20 may be mutated in order to downregulate (not eliminate) its function. As another nonlimiting example, a diploid strain may be used to knockout one copy of ERG20 gene in order to downregulate the activity of the enzyme it encodes. In some cases, an enzyme may not be essential to a chassis and may be knocked out without causing adverse effects. As a nonlimiting example, genes such as MLS1 and CIT1 may be knocked out in such manner.

With continued reference to FIG. 3A-B, in some cases, multiple upregulations and downregulations may be implemented simultaneously to achieve a synergic effect. As a nonlimiting example, gene targets such as zwf, pgl, and/or pkl may be upregulated simultaneously to utilize the Entner-Doudoroff pathway in addition to the Embden-Meyerhof pathway regulated by pgi and pkl, thereby increasing the carbon flux of biosynthesis and increasing the yield of acetylphosphate (see FIG. 3A, embodiment 300a). As another nonlimiting example, gene targets such as ackA-pta, poxB, and/or ldhA may be knocked down or knocked out simultaneously to suppress competing reactions that form acetate, lactate, and/or ethanol (see FIG. 3B, embodiment 300b). As another nonlimiting example, simultaneous upregulation of ALD5 and downregulation of ADH1 may be combined to prevent acetyl-CoA from converting to ethanol via fermentation. As another nonlimiting example, PEX10 may be deleted to redirect carbon flux towards isoprene by impairing peroxisomal function and increasing cytosolic lipid and acetyl-CoA availability. Table 1 summarizes exemplary gene targets for upregulation and downregulation for S. cerevisiae.

TABLE 1
Gene Targets for Improving Isoprene Production in S. cerevisiae.

| | | Type of Genetic | |
|---|---|---|---|
| | | Modification | |
| | | Note: | |
| | | Knockout (KO) | |
| | | Knockdown (KD) | |
| Organism | Gene | Overexpress (OE) | Rationale |
| S. cerevisiae | ERG20: farnesyl | KD or mutate specific | Essential Gene; increase |
| | pyrophosphate synthetase | sites | DMAPP |
| S. cerevisiae | BTS1: geranylgeranyl | KO | Increase DMAPP |
| | diphosphate synthase | | |
| S. cerevisiae | UPC2: transcription factor | OE | Increase products of |
| | | | MVA pathway |
| S. cerevisiae | ERG10: acetyl-CoA C- | OE | Increase products of |
| | acetyltransferase | | MVA pathway |
| S. cerevisiae | ERG12: Mevalonate kinase | OE | Increase products of |
| | | | MVA pathway |
| S. cerevisiae | ERG13: HMG-CoA synthase | OE | Increase products of |
| | | | MVA pathway |
| S. cerevisiae | ALD5: aldehyde | OE | Increase flux from |
| | dehydrogenases | | acetaldehyde to acetyl- |
| | | | CoA instead of ethanol |
| | | | production. Could also |
| | | | work with fed ethanol to |
| | | | push towards acety-CoA |
| S. cerevisiae | ADH1: alcohol dehydrogenase | KO | Increase flux from |
| | | | acetaldehyde to acetyl- |
| | | | CoA instead of ethanol |
| | | | production. Could also |
| | | | work with fed ethanol to |
| | | | push towards acety-CoA |
| S. cerevisiae | ACS1 and ACS2: acetyl-CoA | OE | Increase flux from |
| | synthetase | | acetaldehyde to acetyl- |
| | | | CoA instead of ethanol |
| | | | production. Could also |
| | | | work with fed ethanol to |
| | | | push towards acety-CoA |
| S. cerevisiae | MLS1: Malate synthase | KO | Increase acetyl CoA pool |
| S. cerevisiae | CIT1: Citrate synthase | KO | Increase acetyl CoA pool |
| S. cerevisiae | CAB1: Pantothenate kinase | OE | Increase acetyl CoA pool |
| S. cerevisiae | LAT1: dihydrolipoamide | OE | Increase acetyl CoA pool |
| | acetyltransferase component | | |
| | of the pyruvate dehydrogenase | | |
| | complex | | |
| S. cerevisiae | PEX10: | KO | Redirect carbon flux |
| | Peroxisomal membrane E3 | | towards isoprene |
| | ubiquitin ligase | | |
| E. coli | zwf: glucose-6-phosphate | OE | Increase carbon flux to |
| | dehydrogenase | | acetylphosphate |
| | | | (overexpression of the |
| | | | Entner-Doudoroff |
| | | | pathway) |
| E. coli | acsA: acetyl-coenzyme A | OE | Increase carbon flux |
| | synthetase | | from acetate to acetyl- |
| | | | CoA |
| E. coli | pgl: 6- | OE | Increase carbon flux to |
| | phosphogluconolactonase | | acetylphosphate |
| | (ybhE) | | (overexpression of the |
| | | | Entner-Doudoroff |
| | | | pathway) |
| E. coli | pkl: phosphoketolase | OE | Increase carbon flux to |
| | | | acetylphosphate |
| | | | (overexpression of the |
| | | | Entner-Doudoroff |
| | | | pathway) |
| E. coli | pgi: glucose-6-phosphate | OE | Increase carbon flux |
| | isomerase | | from G6P to F6P |
| E. coli | pta: phosphate | OE | Increase acetyl-CoA |
| | acetyltransferase | | from acetylphosphate |
| E. coli | ldhA: lactate dehydrogenase A | KO/KD | Reduce lactate |
| | | | production from |
| | | | pyruvate |
| E. coli | poxB: pyruvate oxidase | KO/KD | Reduce acetate |
| | | | production from |
| | | | pyruvate |
| E. coli | ackA: acetate kinase | KO/KD | Reduce acetate |
| | | | production from |
| | | | acetylphosphate |
| E. coli | adhE: alcohol dehydrogenase | KO/KD | Reduce ethanol |
| | enzyme | | production from acetyl- |
| | | | CoA |
| E. coli | did: D-lactate dehydrogenase | KO/KD | Reduce lactate |
| | | | production from |
| | | | pyruvate |
| E. coli | aceE: pyruvate dehydrogenase | KD | Increase carbon flux to |
| | | | MEP pathway from |
| | | | pyruvate |
| E. coli | ispA: geranyl diphosphate | KD | Increase carbon flux to |
| | | | DMAPP from IPP and |
| | | | reduce it to GPP from |
| | | | IPP |

With continued reference to FIGS. 3A-B, in one or more embodiments, promoter engineering may be used to improve the translational level of a gene. Promoter engineering modifies the promoter region to increase gene expression levels, thereby improving enzyme production and activity. In one or more embodiments, codon optimization may be used to improve the translational efficiency of a gene. For the purposes of this disclosure, “codon optimization” is a technique used in genetic engineering to improve the expression of a gene in a particular host organism. Codon optimization involves altering the DNA sequence of a gene to use codons that are more frequently preferred by the host organism's translational machinery. A codon optimization process may take into account a codon bias of the host, ensuring that a synthetic gene sequence is translated more efficiently into the desired protein. Codon optimization may enhance the yield and function of a protein, which may be crucial for various applications in biotechnology and synthetic biology. It is worth noting that promoter engineering and codon optimization are distinct yet complementary techniques in genetic engineering. Promoter engineering involves modifying the promoter region of a gene to enhance its expression by improving the binding efficiency of transcriptional machinery. Codon optimization, on the other hand, focuses on altering the coding sequence of a gene to use preferred codons of the host organism, thereby improving translation efficiency. While both aim to increase protein production, promoter engineering targets transcriptional levels, and codon optimization targets translational efficiency. Combining both techniques may in some cases synergistically enhance an overall gene expression.

With continued reference to FIGS. 3A-B, in some cases, a gene encoding an enzyme may be silenced using Hfq-mediated RNA silencing. For the purposes of this disclosure, “Hfq-mediated RNA silencing” is a process wherein the Hfq protein facilitates a regulation of gene expression through RNA interactions, by binding to small regulatory RNAs (sRNAs) and messenger RNAs (mRNAs) to promote a formation of RNA duplexes. This interaction may enhance or inhibit a translation of target mRNAs, leading to gene silencing. In some cases, this process may be useful for post-transcriptional regulation and may be harnessed for genetic engineering, therapeutic interventions, and synthetic biology applications. As nonlimiting examples, to facilitate accumulation of isoprene, Hfq-mediated RNA silencing may be used against geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), malate synthase (encoded by AceB or MLS1), ATP-citrate lyase (CitE) including AclY, Ac1B, and Ac1A, and/or pyruvate dehydrogenase (encoded by PDH) including pyruvate dehydrogenase E1 component (encoded by AceE), among others, to downregulate its activity, consistent with details described above.

With continued reference to FIGS. 3A-B, in some cases, one or more pathways within schemes of embodiments 300a or 300b may be condensed, inhibited, rearranged, or otherwise modified. As a nonlimiting example, one or more genes encoding one or more enzymes may be modified to provide a phosphoketolase workaround, such as by upregulating glycolysis, the pentose phosphate pathway, or the Entner-Doudoroff pathway. As another nonlimiting example, one or more enzymes may be co-localized to create multiple reaction centers in tandem, thereby bypassing one or more competing side reactions, consistent with details described above and disclosed in U.S. Pat. App. Ser. No. 63/713,890 (attorney docket number 1656-002USP1), filed on Oct. 30, 2024, entitled “GENETICALLY ENGINEERED PEPTIDE SUPERSTRUCTURE AND MICROBE FOR PRODUCTION OF ISOPRENE AND METHOD OF PRODUCTION THEREOF”, the entirety of which is incorporated herein by reference.

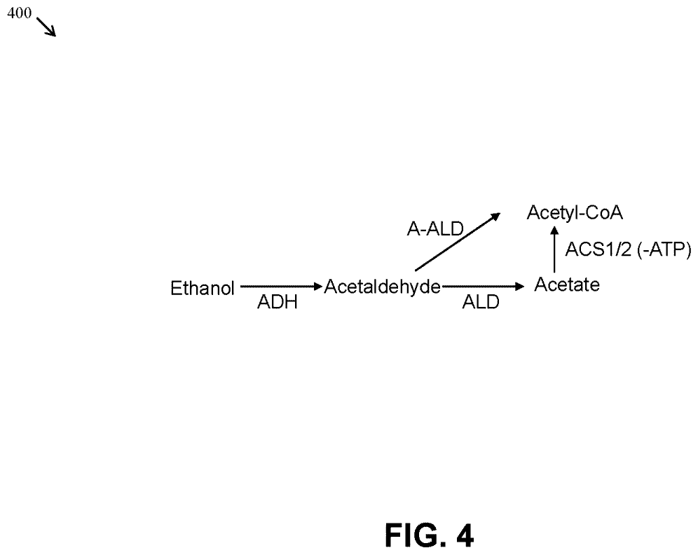

Referring now to FIG. 4, scheme 400 illustrates an exemplary embodiment of an engineered metabolic pathway pertaining to biosynthesis of isoprene. To maximize production of isoprene from ethanol, one or more alternative metabolic pathways may be constructed. In some cases, expression of genes encoding certain enzymes such as ACS and ERG10 (the entry points for terpene pathways such as the MVA pathway) may be controlled. In some cases, a heterologous nucleic acid construct including an A-ALD gene may be introduced to a genetically engineered microbe to produce A-ALD at least partially in place of ACS and/or acetaldehyde dehydrogenase (ALD). As nonlimiting examples, native ACS1 and/or ACS2 genes may be eliminated. By removing ACS activity while culturing a genetically engineered microbe on ethanol, a competing, less efficient pathway that converts acetaldehyde to acetyl-CoA may be suppressed or eliminated. The rationale for eliminating ACS in favor of A-ALD is to have a route to acetyl-CoA in the cytosol that does not require ATP. ACS requires ATP, but A-ALD doesn't. This design may improve yield of isoprene and/or terpenes and likely make the genetically engineered microbe more robust i.e., capable of replicating and/or producing isoprene and/or terpenes at a faster speed. This may be achieved by controlling the expression of a gene encoding ACS and/or the activity of ACS with an HXT1promoter driving ACS1, which is induced by glucose and repressed in its absence. Conversely, A-ALD expression may be repressed in the presence of glucose and induced in its absence, such as via an ADH2 promoter, allowing for the conversion of acetaldehyde directly to A-CoA. ERG10 may be expressed using the same ADH2 promoter, ensuring a high level of its expression with ethanol as the carbon source and a high concentration of cytosolic A-CoA to initiate downstream isoprene/terpene production.

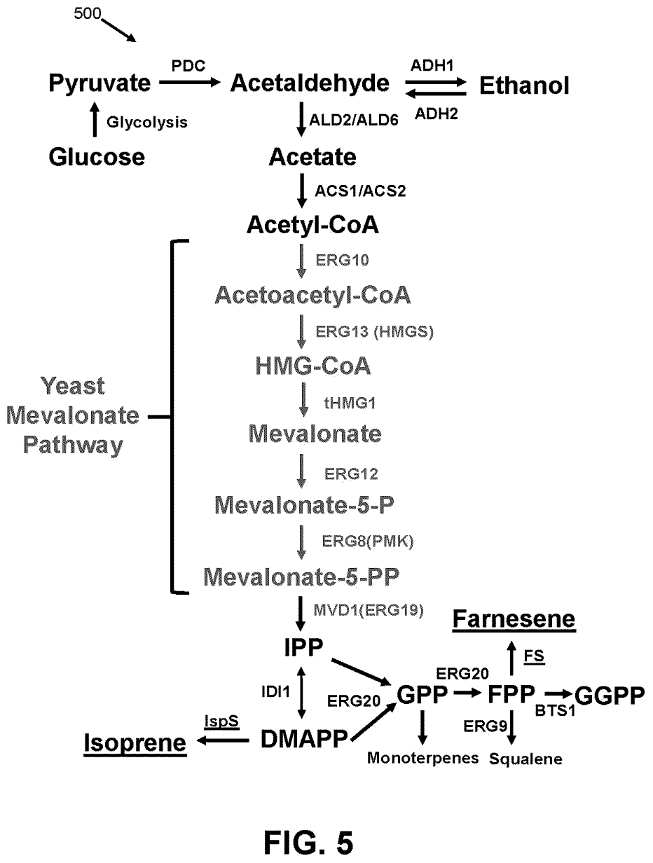

Referring now to FIG. 5, scheme 500 illustrates an exemplary embodiment of yeast mevalonate pathway and downstream pathways for conversion to isoprene or farnesene by introduction of IspS or farnesene synthase (FS) respectively. In one or more embodiments, a genetically engineered yeast cell may metabolize a carbon source such as glucose or ethanol into isoprenoid precursors via endogenous enzymatic processes. In an embodiment, glucose may be first metabolized through glycolysis to form pyruvate, which may be converted into acetaldehyde by pyruvate decarboxylase (PDC). Acetaldehyde may be reduced to ethanol via alcohol dehydrogenase 1 (ADH1) or may be oxidized to acetate by aldehyde dehydrogenase isoforms ALD2 or ALD6. Acetate may be converted to acetyl coenzyme A (acetyl-CoA) via acetyl-CoA synthetase (ACS1 or ACS2). In an embodiment, ethanol may be first metabolized through alcohol dehydrogenase 2 (ADH2) to form acetaldehyde. Acetaldehyde may be reduced to ethanol via alcohol dehydrogenase 1 (ADH1) or may be oxidized to acetate by aldehyde dehydrogenase isoforms ALD2 or ALD6. Acetate may be converted to acetyl coenzyme A (acetyl-CoA) via acetyl-CoA synthetase (ACS1 or ACS2). Acetyl-CoA may serve as a precursor for the yeast mevalonate pathway.

With continued reference to FIG. 5, yeast mevalonate pathway may include a series of enzymatic conversions. In one or more embodiments, acetyl-CoA may be converted into Acetoacetyl-CoA by acetyl-CoA C-acetyltransferase (ERG10). Acetoacetyl-CoA may be followed by conversion to 3-hydroxy-3-methylglutaryl coenzyme A (HMG-COA) via hydroxymethylglutaryl-CoA synthase (ERG13, also referred to as HMGS). HMG-COA may be subsequently converted into mevalonate by HMG-COA reductase (HMG1). In some embodiments, HMG1 may be a rate-limiting step in the yeast mevalonate pathway. In some embodiments, a truncated form of HMG-COA reductase (tHMG1), lacking a portion of the N-terminal regulatory domain, may be used in yeast mevalonate pathway. Mevalonate may be subsequently converted to mevalonate-5-phosphate (Mevalonate-5-P) by mevalonate kinase (ERG12). Mevalonate-5-P may be converted into mevalonate-5-pyrophosphate (Mevalonate-5-PP) via phosphomevalonate kinase (ERG8, also referred to as PMK). Mevalonate-5-PP may be subsequently converted into isopentenyl pyrophosphate (IPP) via diphosphomevalonate decarboxylase (MVD1, also referred to as ERG19).

With continued reference to FIG. 5, in one or more embodiments, IPP may be converted to dimethylallyl pyrophosphate (DMAPP) by IPP isomerase (IDI1). In an embodiments, IPP and DMAPP may be converted by farnesyl pyrophosphate synthase (ERG20) to generate geranyl pyrophosphate (GPP). GPP may be subsequently converted into farnesyl pyrophosphate (FPP) via ERG20. In one or more embodiments, FPP may follow different downstream branches: (i) FPP may be converted into squalene by squalene synthase (ERG9), or (ii) FPP may be converted into geranylgeranyl pyrophosphate (GGPP) by GGPP synthase (BTS1), or (iii) FPP may be converted into farnesene via farnesene synthase (FS). In one or more embodiments, an expression of isoprene synthase (IspS) may converted DMAPP into isoprene. In some embodiments, IspS may be codon-optimized or modified for amino acids substitution or or truncations such as exemplary IspS modifications: IspSm or IspSf. As shown in FIG. 5, non-native enzymatic steps, including IspS or FS mediated conversions, are depicted in outlined font.

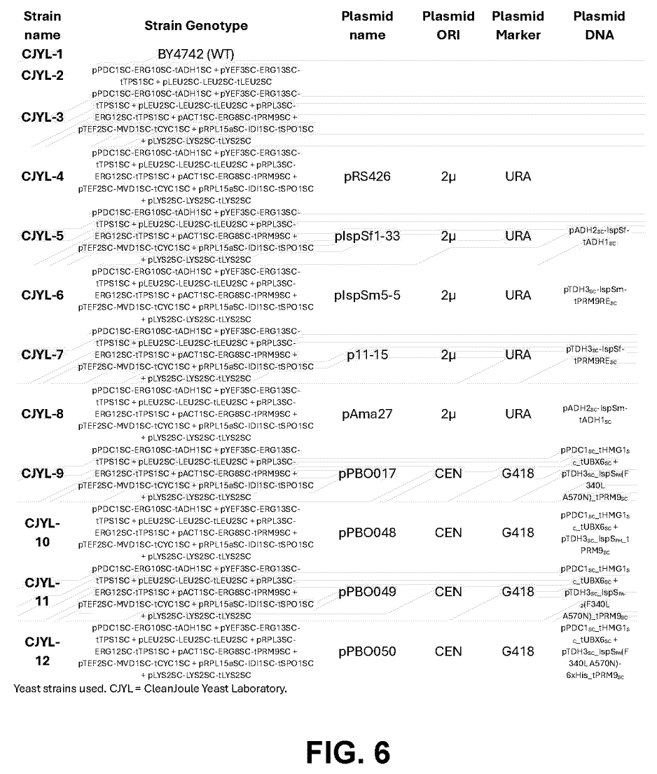

Referring now to FIG. 6, shown is a table of constructed strains of yeast with genetically modification. In one or more embodiments, a strain of yeast may be constructed with two chromosomal integrations: pYI33 at the Delta22 and site, and pY131 and pY132 at the Delta 15 site. pY133 may introduce a single copy each of pPDC1SC-ERG10SC-tADH1SC, pYEF3SC-ERG13SC-tTPS1SC, and pLEU2SC-LEU2SC-tLEU2SC. pYI31 and pYI32 may introduce a single copy each of pRPL3SC-ERG12SC-tTPS1SC, pACT1SC-ERG8SC-tPRM9SC, pTEF2SC-MVD1SC-tCYC1SC, pRPL15aSC-IDI1SC-tSPO1SC, and pLYS2SC-LYS2SC-tLYS2SC (using a split LYS2 fragment on both DNA fragments for their simultaneous integration). This base strain may then transformed with 2μ URA plasmids expressing a variant of Populus alba IspS (either F340L & A570N version, “IspSm”, or a truncated version of the mutant with a 52 amino acid N-terminal portion removed and two different mutations S339C and G542S, “IspSf”) under control of either the ADH2 or TDH3 promoter, or the negative control pRS426 plasmid. Notably, these strains may not require to have overexpression of HMG1.

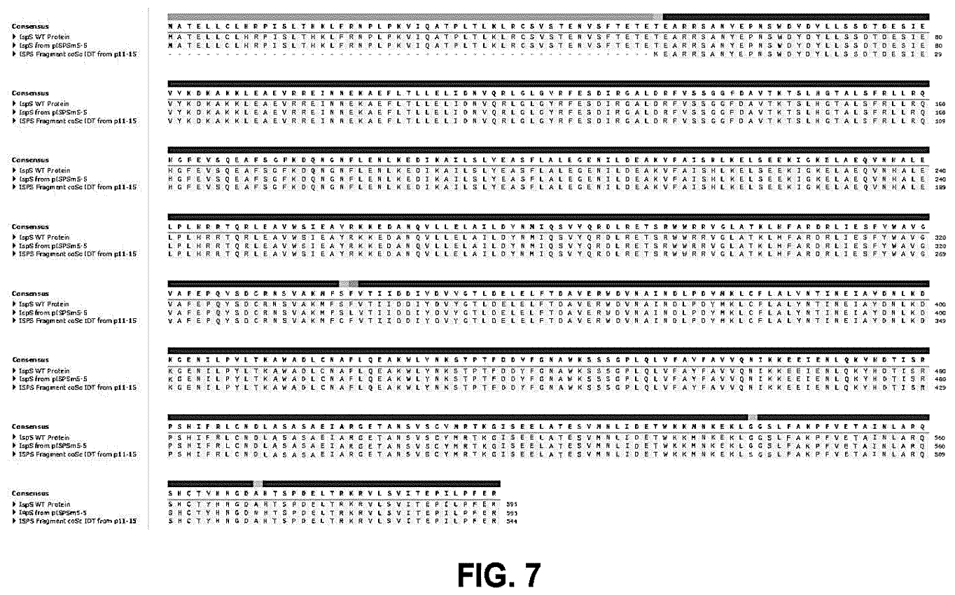

Referring now to FIG. 7, shown is an alignment of the ISPS protein sequences against the wild type ISPS sequence from P. alba. In one or more embodiments, the base strain may be transformed with CEN G418 plasmids expressing both truncated yeast HMG1 (tHMG1) and various IspS sequences: P. alba IspS with two mutations (F340L and A570N) (pPBO017). In one or more embodiments, the same IspS codon may be optimized differently (pPBO049) or His-tagged (pPBO050), and an IspS from the Kudzu vine Pueraria montana (pPBO048).

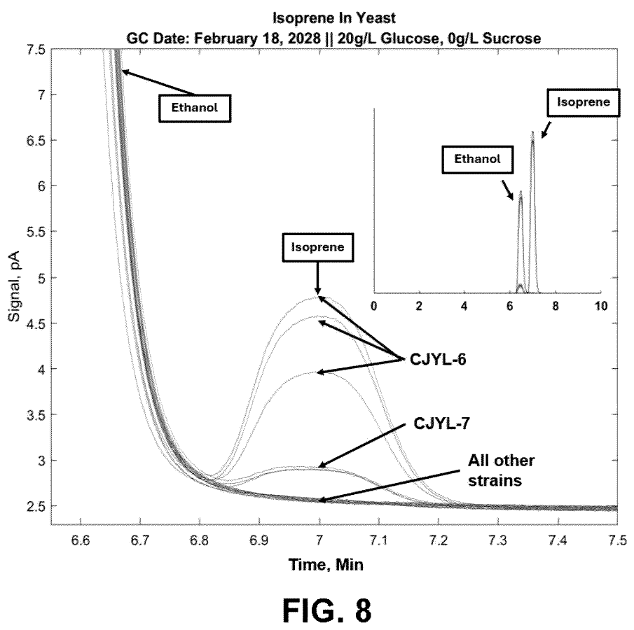

Referring now to FIG. 8, shown is a raw gas chromatography (GC) traces of samples from headspace of all Yest Isoprene Production Screen 2 (YIPS_2) cultures, zoomed in on the retention time expected for isoprene based on standards which is inlayed in the top right corner. In one or more embodiments, a panel of genetically engineered yeast strains, as listed in FIG. 6, were evaluated across five independent isoprene biosynthesis screening experiments, designated as Yest Isoprene Production Screen (YIPS_2 through YIPS_6). In YIPS_2, strains harboring high copy number (2-micron origin) URA3-based plasmids, including strains CJYL-4 through CJYL-8, were grown in synthetic defined media lacking uracil (SD-URA) and supplemented with 20 g/L glucose as the carbon source. CJYL-1 strain was grown in synthetic defined media (SD). In certain embodiments, a yeast fermentation process may result in endogenous ethanol product, which required adjustments of GC parameters to resolve closely eluting two separate compounds (ethanol and isoprene), thereby a longer GV run time may be employed. Under this GC protocol, an ethanol peak may elute at approximately 6.5 minutes, while a isoprene peak may be elute at approximately 7.0 minutes.

With continued reference to FIG. 8, shown is a GC obtained from analysis of yeast culture samples. The x-axis of the chromatogram represents the retention time, measured in minutes. The y-axis represents the detector signal, measured in picoamperes (pA), corresponding to the ionization response generated by the flame ionization detector (FID). In one or more embodiments, a distinct peak observed at approximately 7.0 minutes corresponds to isoprene. The amplitude of the signal in pA unites reflects the relative abundance of isoprene in the sample. Strains CJYL-6 and CJYL-7 exhibited a distinct GC peak at approximately 7.0 minutes, corresponding to the retention time of isoprene. CJYL06 contains plasmid pIspSm5-5 and CJYL-7 contains plasmid p11-15. Both strains encode engineered isoprene synthase (IspS) variants under yeast-compatible expression system. All other strains did not exhibit distinguishable peak at approximately 7.0 minutes for isoprene.

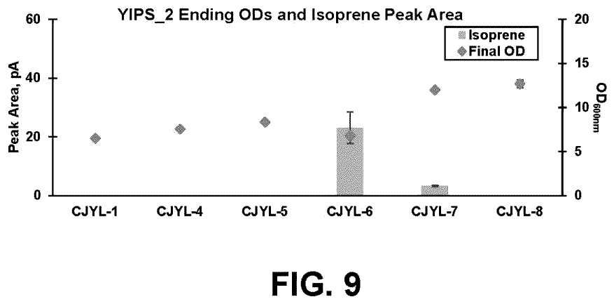

Referring now to FIG. 9, shown is a bar graph of final optical density (OD600 nm) and corresponding isoprene peak area (pA) for various engineered yeast strains following the YIPS_2 assay. Error bars represent standard deviations of biological triplicates. The x-axis indicates the tested strains, including CJYL-1, CJYL-4, CJYL-5, CJYL-6, CJYL-7, CJYL-8. The left y-axis indicates the isoprene peak area measured in picoamperes (pA), as detected by gas chromatograph. The right y-axis indicates the optical density of 600 nanometers (OD600 nm), representing the final culture density at the end of fermentation. Isoprene is represented by bar with solid fill pattern and final OD is represented by rhombus with solid fill pattern. As shown in FIG. 9, strains CJYL-6 and CJYL-7 exhibited both growth and an elevated isoprene peak relative to other strains with no detectable and quantifiable isoprene (CJYL-1, CJYL-4, CJYL-5, CJYL-8).

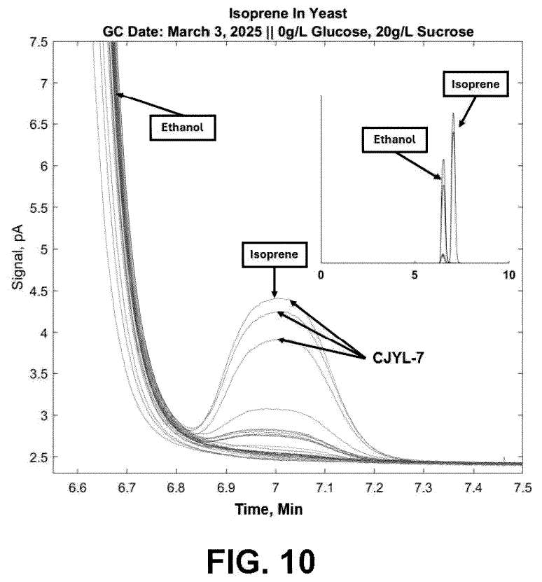

Referring now to FIG. 10, shown is a raw gas chromatography (GC) traces of samples from headspace of all Yest Isoprene Production Screen 3 (YIPS_3) cultures, zoomed in on the retention time expected for isoprene based on standards which is inlayed in the top right corner. In one or more embodiments, a panel of genetically engineered yeast strains, as listed in FIG. 6, were evaluated across five independent isoprene biosynthesis screening experiments, designated as Yest Isoprene Production Screen (YIPS_2 through YIPS_6). In YIPS_3, strains harboring high copy number (2-micron origin) URA3-based plasmids, including strains CJYL-4 through CJYL-8, were grown in synthetic defined media lacking uracil (SD-URA) and supplemented with 20 g/L glucose as the carbon source. CJYL-1 strain was grown in synthetic defined media (SD) and supplemented with 20 g/L glucose as the carbon source. CJYL-9 through CJYL-12, were grown in synthetic defined minimal media without leucine or lysine and with G418 supplementation. In certain embodiments, a yeast fermentation process may result in endogenous ethanol product, which required adjustments of GC parameters to resolve closely eluting two separate compounds (ethanol and isoprene), thereby a longer GV run time may be employed. Under this GC protocol, an ethanol peak may elute at approximately 6.5 minutes, while an isoprene peak may be eluted at approximately 7.0 minutes.

With continued reference to FIG. 10, shown is a GC obtained from analysis of yeast culture samples. The x-axis of the chromatogram represents the retention time, measured in minutes. The y-axis represents the detector signal, measured in picoamperes (pA), corresponding to the ionization response generated by the flame ionization detector (FID). In one or more embodiments, a distinct peak observed at approximately 7.0 minutes corresponds to isoprene. The amplitude of the signal in pA unites reflects the relative abundance of isoprene in the sample. Strains CJYL-6 and CJYL-7 exhibited a distinct GC peak at approximately 7.0 minutes, corresponding to the retention time of isoprene. CJYL06 contains plasmid plspSm5-5 and CJYL-7 contains plasmid p11-15. Additionally, strains CJYL-10 (with the P. montana IspS) and CJYL-11 (with new codon optimization of mutant P. alba IspS) also produced measurable isoprene at similar traced level to CJYL-6. All other strains did not exhibit distinguishable peak at approximately 7.0 minutes for isoprene.

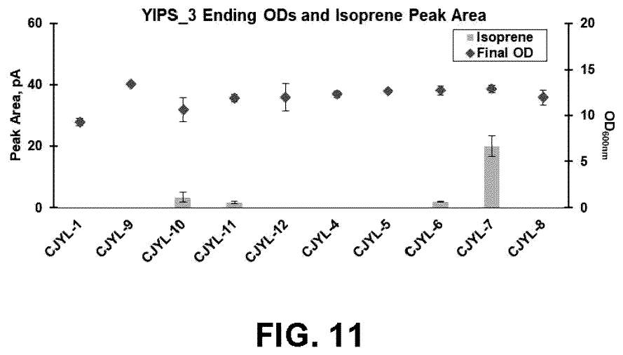

Referring now to FIG. 11, shown is a bar graph of final optical density (OD₆₀₀ₙₘ) and corresponding isoprene peak area (pA) for various engineered yeast strains following the YIPS_3 assay. Error bars represent standard deviations of biological triplicates. The x-axis indicates the tested strains, including CJYL-1, CJYL-4, CJYL-5, CJYL-6, CJYL-7, CJYL-8, CJYL-9, CJYL-10, CJYL-11, CJYL-12. The left y-axis indicates the isoprene peak area measured in picoamperes (pA), as detected by gas chromatograph. The right y-axis indicates the optical density of 600 nanometers (OD600 nm), representing the final culture density at the end of fermentation. Isoprene is represented by bar with solid fill pattern and final OD is represented by rhombus with solid fill pattern. As shown in FIG. 11, strains CJYL-6, CJYL-7, CJYL-10, CJYL-11 exhibited both growth and an elevated isoprene peak relative to other strains with no detectable and quantifiable isoprene (CJYL-1, CJYL-4, CJYL-5, CJYL-8, CJYL-9, CJYL-12).

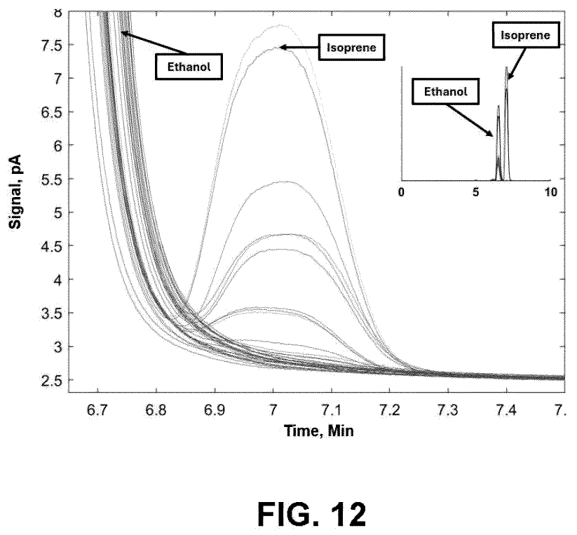

Referring now to FIG. 12, shown is a raw gas chromatography (GC) traces of samples from headspace of all Yest Isoprene Production Screen 4 (YIPS_4) cultures, zoomed in on the retention time expected for isoprene based on standards which is inlayed in the top right corner. In one or more embodiments, a panel of genetically engineered yeast strains, as listed in FIG. 6, were evaluated across five independent isoprene biosynthesis screening experiments, designated as Yest Isoprene Production Screen (YIPS). In YIPS_4, strains harboring high copy number (2-micron origin) URA3-based plasmids, including strains CJYL-4, CJYL-6, and CJYL-7, were grown in synthetic defined media lacking uracil (SD-URA). CJYL-1 strain was grown in synthetic defined media (SD) and supplemented with 20 g/L glucose as the carbon source. CJYL-9 and CJYL-10, were grown in synthetic defined minimal media without leucine or lysine and with G418 supplementation. There were two type of carbon source for growth: (i) media was supplemented with 50 g/L glucose; (ii) media was supplemented with 4% ethanol and 3% (wt/vol) glycerol. In certain embodiments, a yeast fermentation process may result in endogenous ethanol product, which required adjustments of GC parameters to resolve closely eluting two separate compounds (ethanol and isoprene), thereby a longer GV run time may be employed. Under this GC protocol, an ethanol peak may elute at approximately 6.5 minutes, while an isoprene peak may be elute at approximately 7.0 minutes.

With continued reference to FIG. 12, shown is a GC obtained from analysis of yeast culture samples. The x-axis of the chromatogram represents the retention time, measured in minutes. The y-axis represents the detector signal, measured in picoamperes (pA), corresponding to the ionization response generated by the flame ionization detector (FID). In one or more embodiments, a distinct peak observed at approximately 7.0 minutes corresponds to isoprene. The amplitude of the signal in pA unites reflects the relative abundance of isoprene in the sample. Strains CJYL-6 exhibited a distinct GC peak at approximately 7.0 minutes in glucose only as carbon source, corresponding to the retention time of isoprene. Strains CJYL-7 exhibited a distinct GC peak at approximately 7.0 minutes in both glucose and ethanol/glycerol as carbon source, corresponding to the retention time of isoprene. CJYL06 contains plasmid pIspSm5-5 and CJYL-7 contains plasmid p11-15. All other strains did not exhibit distinguishable peak at approximately 7.0 minutes for isoprene.

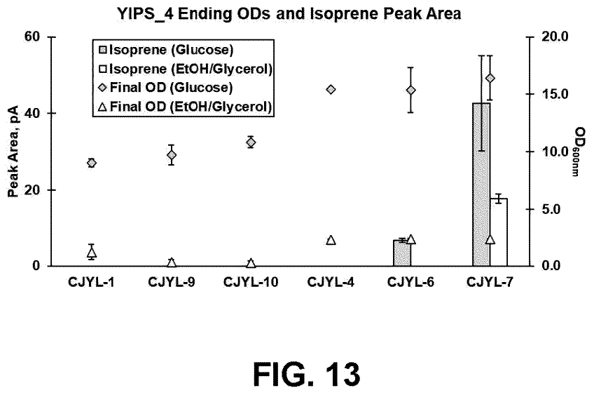

Referring now to FIG. 13, shown is a bar graph of final optical density (OD600 nm) and corresponding isoprene peak area (pA) for various engineered yeast strains following the YIPS_4 assay. Error bars represent standard deviations of biological triplicates. The x-axis indicates the tested strains, including CJYL-1, CJYL-4, CJYL-6, CJYL-7, CJYL-9, CJYL-10. The left y-axis indicates the isoprene peak area measured in picoamperes (pA), as detected by gas chromatograph. The right y-axis indicates the optical density of 600 nanometers (OD600 nm), representing the final culture density at the end of fermentation. Isoprene (Glucose) is represented by bar with solid fill pattern, isoprene (EtOH/Glycerol) is represented by bar with no fill pattern, final OD (glucose) is represented by rhombus with solid fill pattern, final OD (EtOH/Glycerol) is represented by triangle with no fill. In general, growth of strains was lower in ethanol/glycerol supplemented media compared to glucose supplemented media As shown in FIG. 13, strains CJYL-6 (glucose only), CJYL-7 (glucose and ethanol/glycerol), exhibited both growth and an elevated isoprene peak relative to other strains with no detectable and quantifiable isoprene (CJYL-1, CJYL-4, CJYL-9, CJYL-10).

Referring now to FIG. 13 shown is a raw gas chromatography (GC) traces of samples from headspace of all Yest Isoprene Production Screen 5 (YIPS_5) cultures. In one or more embodiments, a panel of genetically engineered yeast strains, as listed in FIG. 6, were evaluated across five independent isoprene biosynthesis screening experiments, designated as Yest Isoprene Production Screen (YIPS). In YIPS_5, strains harboring high copy number (2-micron origin) URA3-based plasmids, including strains CJYL-4, CJYL-6, and CJYL-7, were grown in synthetic defined media lacking uracil (SD-URA) with an adaptation step for 3% (wt/vol) glycerol. CJYL-1 strain was grown in Yeast Peptone Dextrose (YPD) and supplemented with Yeast Peptone Glycerol (YPG) which contains 3% (wt/vol) glycerol or YPEG supplement which is a yeast extract, peptone medium supplemented with 3% (wt/vol) ethanol and 3% (wt/vol) glycerol as the carbon source. CJYL-9 and CJYL-10, were grown in YPG medium with G418 supplementation. There were six types of carbon source for growth: (i) CJYL-1 was grown in either YPG or YPEG medium; (ii) CJYL-4, CJYL-6, CJYL-7 were grown in either SD-URA with 3% (wt/vol) glycerol or 3% (wt/vol) ethanol and 3% (wt/vol) glycerol; (iii) CJYL-9, CJYL-10 were grown in either YPG with G418 or YPEG with G418. In certain embodiments, a yeast fermentation process may result in endogenous ethanol product, which required adjustments of GC parameters to resolve closely eluting two separate compounds (ethanol and isoprene), thereby a longer GV run time may be employed. Under this GC protocol, an ethanol peak may elute at approximately 6.5 minutes, while an isoprene peak may be elute at approximately 7.0 minutes.

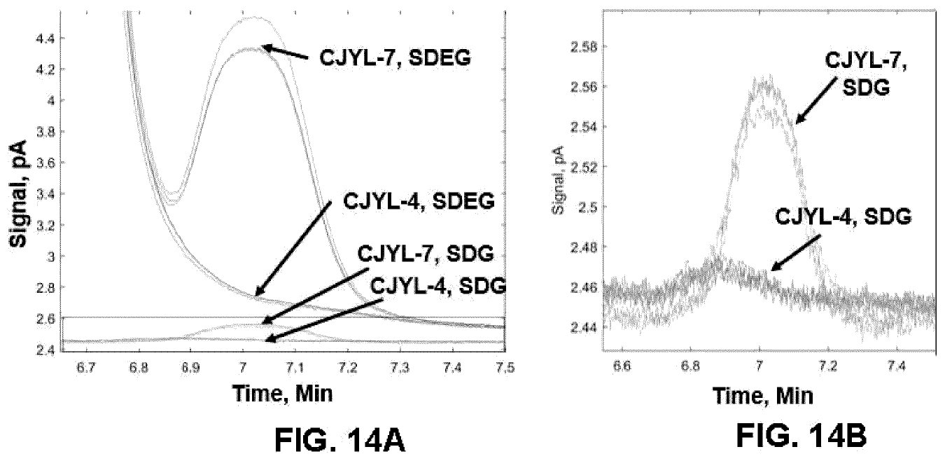

With continued reference to FIGS. 14A-B, FIG. 14A shown is a GC obtained from analysis of yeast culture samples. The x-axis of the chromatogram represents the retention time, measured in minutes. The y-axis represents the detector signal, measured in picoamperes (pA), corresponding to the ionization response generated by the flame ionization detector (FID). In one or more embodiments, a distinct peak observed at approximately 7.0 minutes corresponds to isoprene. The amplitude of the signal in pA unites reflects the relative abundance of isoprene in the sample. Strains CJYL-7 exhibited a distinct GC peak at approximately 7.0 minutes in both glycerol and ethanol/glycerol as carbon source, corresponding to the retention time of isoprene. All other strains did not exhibit distinguishable peak at approximately 7.0 minutes for isoprene. FIG. 14B shown is zoom in GC traces on the glycerol only condition in CJYL-4 and CJYL-7.

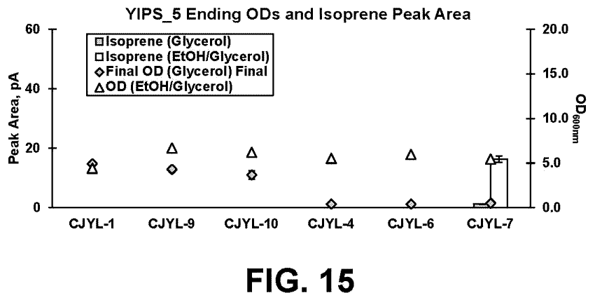

Referring now to FIG. 15, shown is a bar graph of final optical density (OD600 nm) and corresponding isoprene peak area (pA) for various engineered yeast strains following the YIPS_5 assay. Error bars represent standard deviations of biological triplicates. The x-axis indicates the tested strains, including CJYL-1, CJYL-4, CJYL-6, CJYL-7, CJYL-9, CJYL-10. The left y-axis indicates the isoprene peak area measured in picoamperes (pA), as detected by gas chromatograph. The right y-axis indicates the optical density of 600 nanometers (OD600 nm), representing the final culture density at the end of fermentation. Isoprene (Glucose) is represented by bar with solid fill pattern, isoprene (EtOH/Glycerol) is represented by bar with no fill pattern, final OD (glucose) is represented by rhombus with solid fill pattern, final OD (EtOH/Glycerol) is represented by triangle with no fill. There was little growth for CJYL-4, CJYL-6, CJYL-7 strains in the glycerol only condition. There was a detectable amount of isoprene in the glycerol only condition and ethanol/glycerol condition for CJYL-7. Other strains had no detectable and quantifiable isoprene (CJYL-1, CJYL-4, CJYL-9, CJYL-10, CJYL-6).

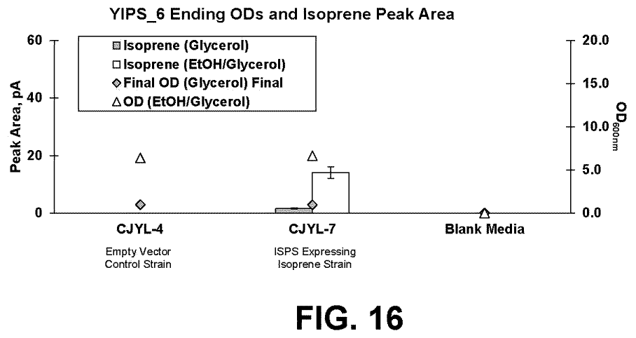

Referring now to FIG. 16, shown is a bar graph of final optical density (OD₆₀₀ₙₘ) and corresponding isoprene peak area (pA) for various engineered yeast strains following the YIPS_6 assay. Error bars represent standard deviations of biological triplicates. The x-axis indicates the tested strains, including CJYL-4, CJYL-7. The left y-axis indicates the isoprene peak area measured in picoamperes (pA), as detected by gas chromatograph. The right y-axis indicates the optical density of 600 nanometers (OD600 nm), representing the final culture density at the end of fermentation. Isoprene (Glucose) is represented by bar with solid fill pattern, isoprene (EtOH/Glycerol) is represented by bar with no fill pattern, final OD (glucose) is represented by rhombus with solid fill pattern, final OD (EtOH/Glycerol) is represented by triangle with no fill. There was little growth for CJYL-4, CJYL-7 strains in the glycerol only condition. There was a detectable amount of isoprene in the glycerol only condition and ethanol/glycerol condition for CJYL-7. In CJYL-7, ethanol/glycerol growth condition had significantly more isoprene production than glycerol only condition. CJYL-4 strains had no detectable and quantifiable isoprene. CJYL-4 was used as a control strain with no vector for expression. Blank media was used as control for isoprene detection.

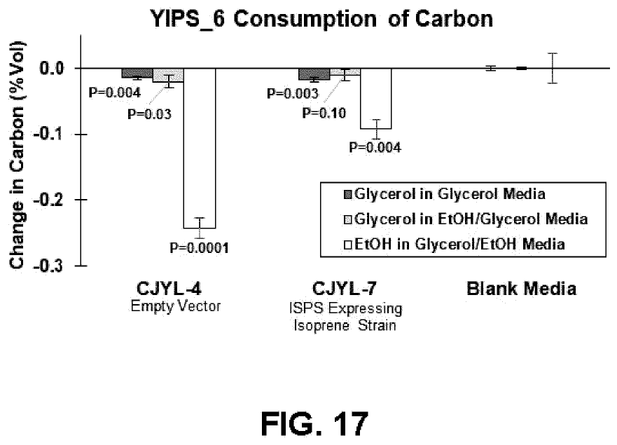

Referring now to FIG. 17, shown is a bar graph of change in carbon (% Vol) for various engineered yeast strains following the YIP_6 assay. Consumption of carbon during YIPS_6 assay were quantified by high pressure liquid chromatograph (HPLC) analysis of samples at the experiment compared to technical triplicates of blank media processed identically to cultures. Error bars represent standard deviation of biological or technical triplicates. P-values were calculated using a two-tailed type-II student's T-test. The x-axis represents samples used for YIP_6 assay. The y-axis represents change in carbon as represent by decrease in % volume. Glycerol in glycerol media is represented by bar with darkest filling. Glycerol in ethanol/glycerol media is represented by bar with medium shade filling. Ethanol in glycerol/ethanol media is represented by bar with no filling. CJYL-7 and CJYL-4 strains showed significant decrease in ethanol in glycerol/ethanol media

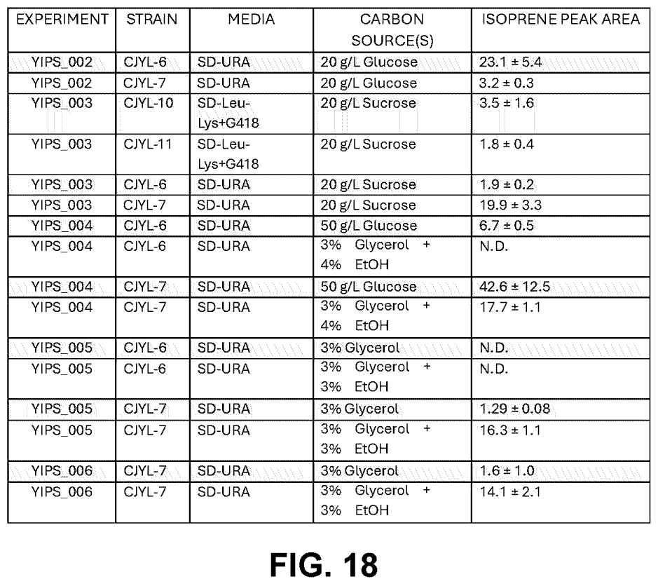

Referring now to FIG. 18, shown is a table of a compilation of isoprene peak areas identified across experimental assays from YIPS_002 to YIPS_006. N.D. stands for not detected.

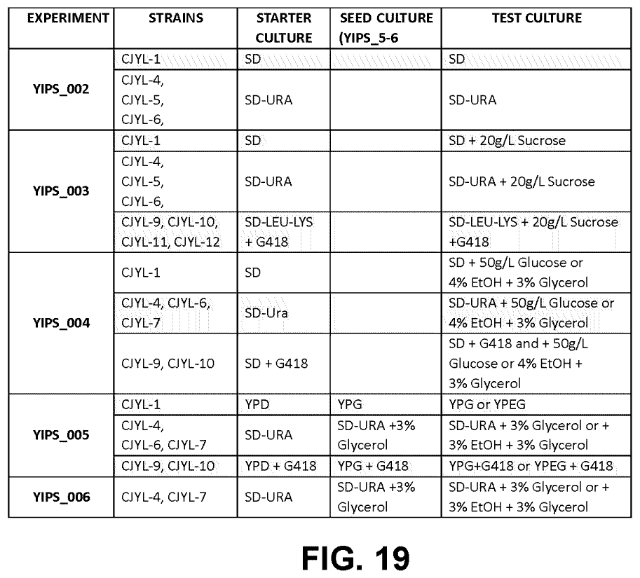

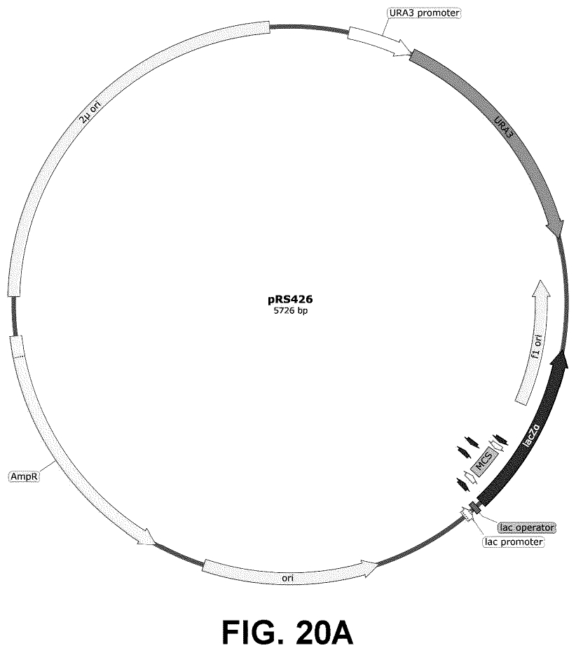

Referring now to FIG. 19, is a table of strains and conditions evaluated in experiments YIPS_2-6. Selective markers such as the necessary antibiotics or auxotrophic markers are not indicated in the media listed above.

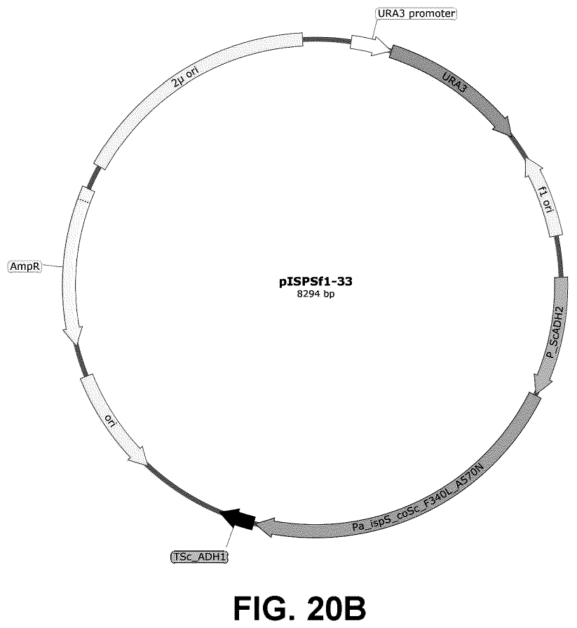

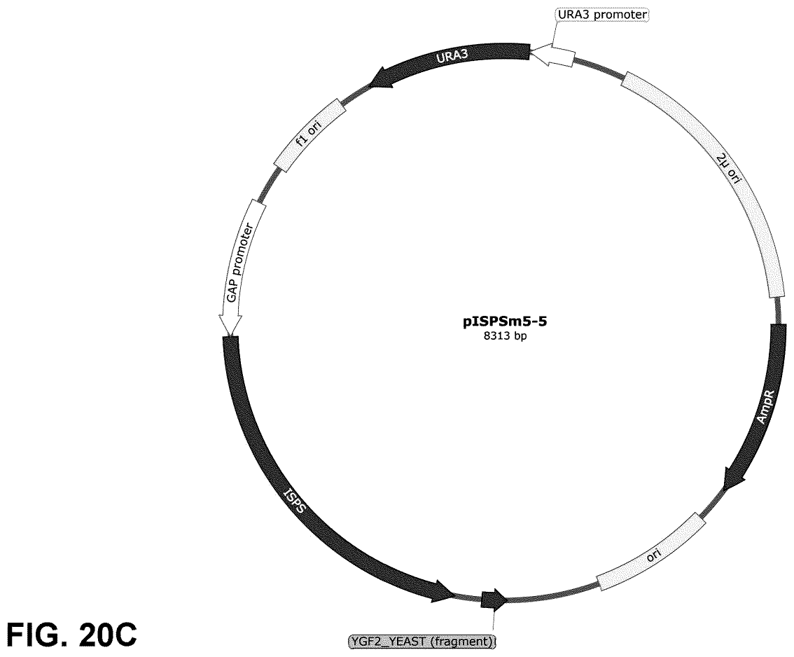

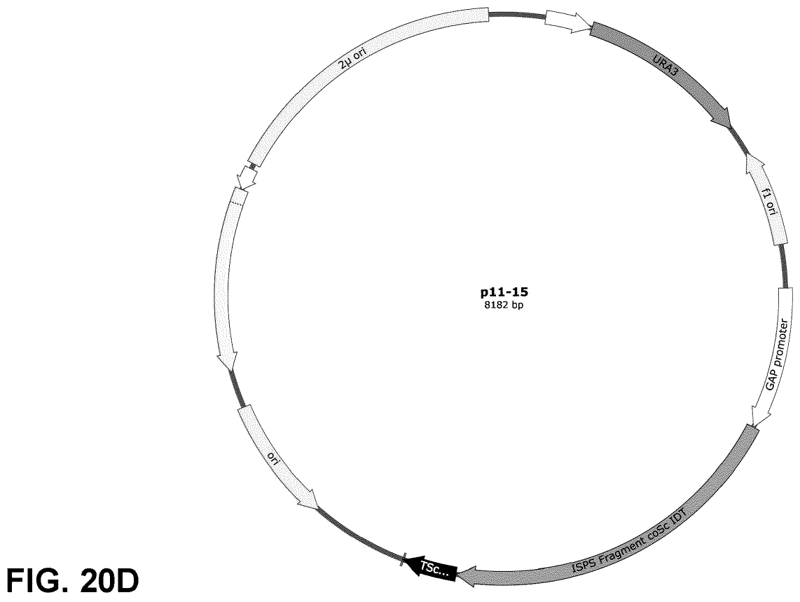

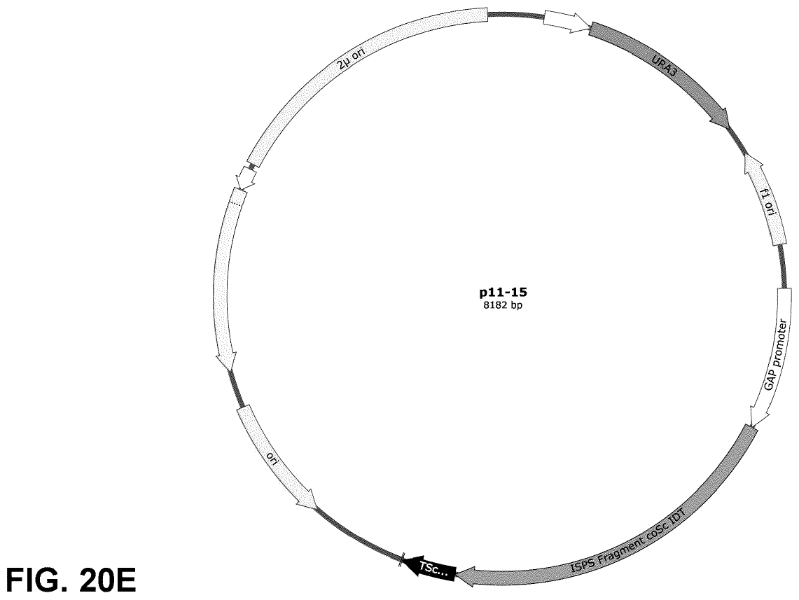

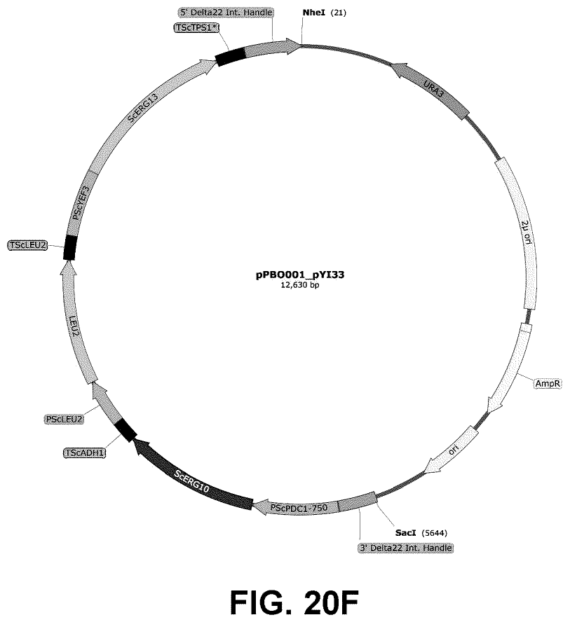

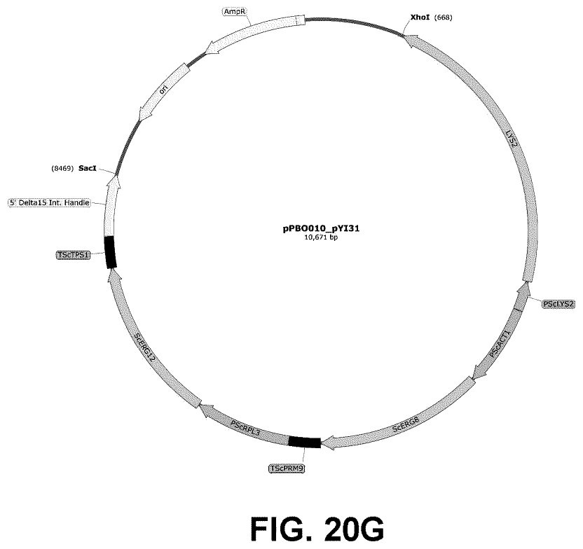

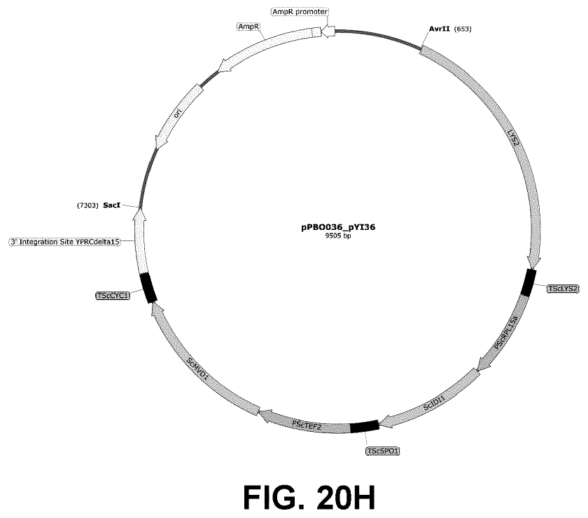

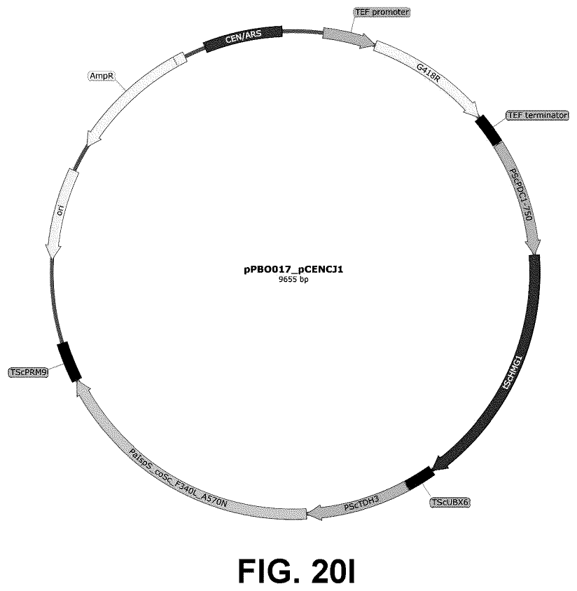

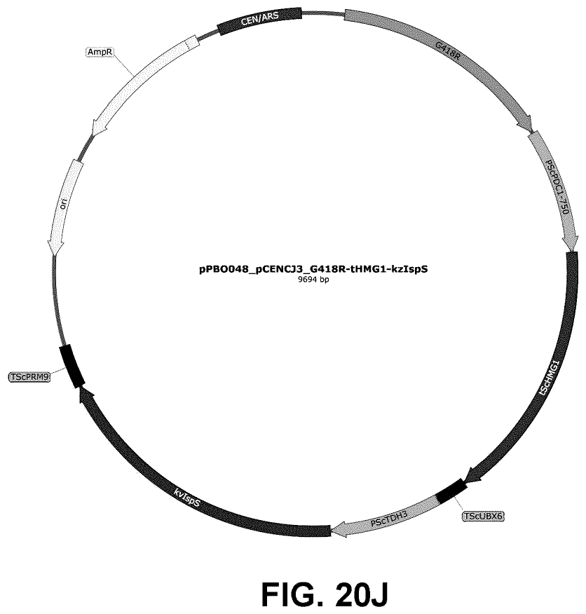

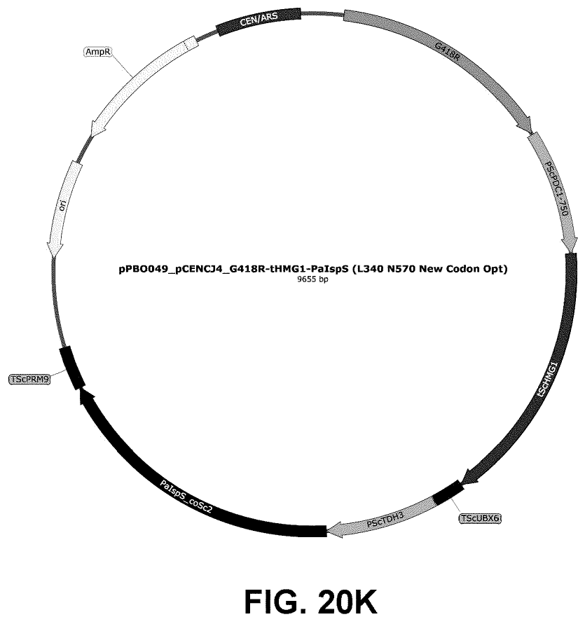

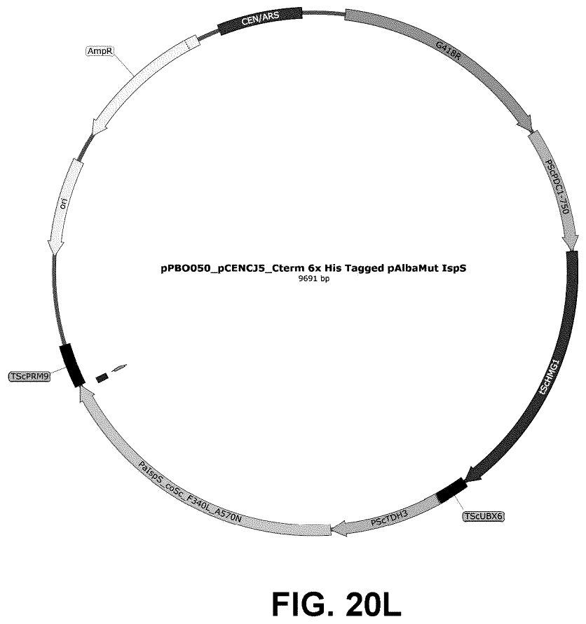

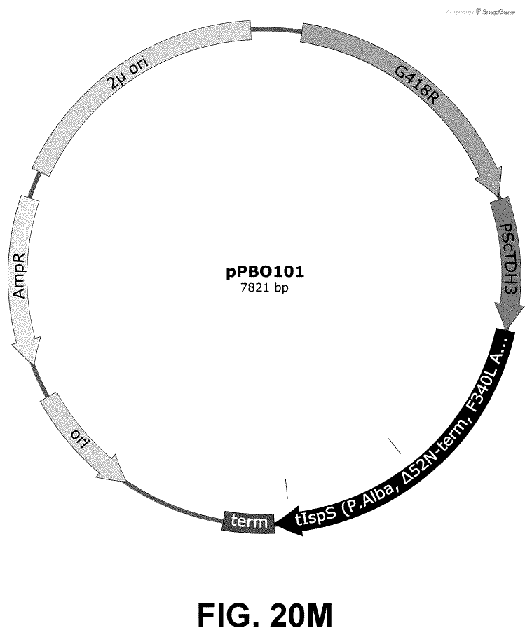

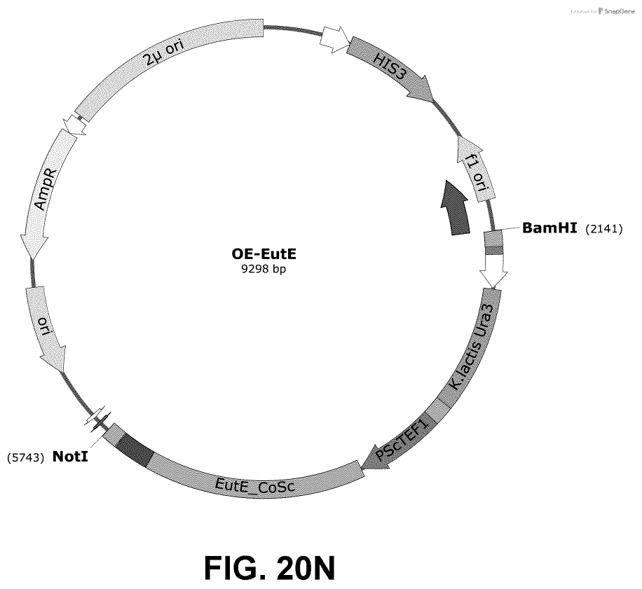

Referring now to FIGS. 20A-N, are exemplary embodiments of plasmid vector maps used for experiments.

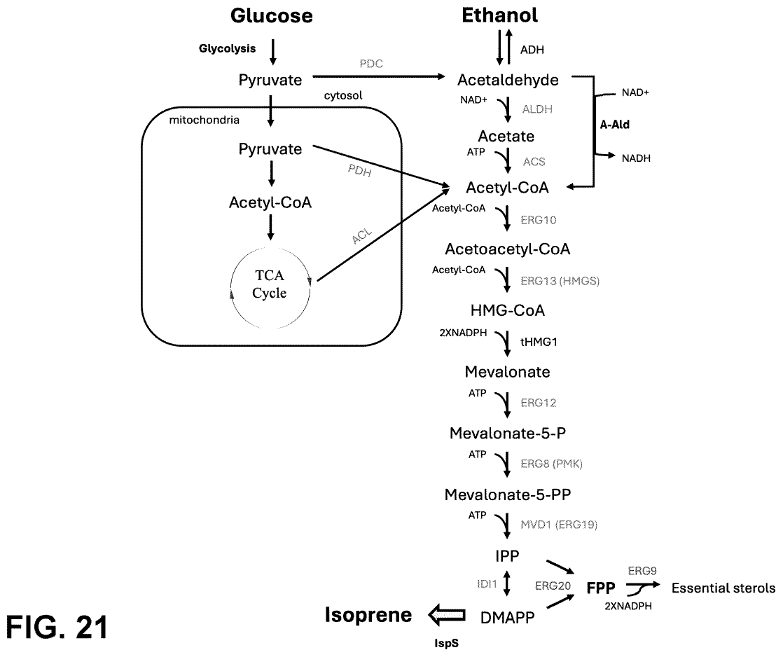

Referring now to FIG. 21, a scheme of an exemplary embodiment of a Native mevalonate (MVA) pathway in yeast with the upstream pathways for synthesis of Acetyl-CoA and downstream pathways for conversion to isoprene by introduction of IspS is illustrated. Native flux to essential sterols with Erg20 and Erg9 is also shown. tHMG1=truncated HMG1, which is indicated as the rate limiting step for MVA flux in yeast and is improved by truncating an N-terminal portion of the protein.

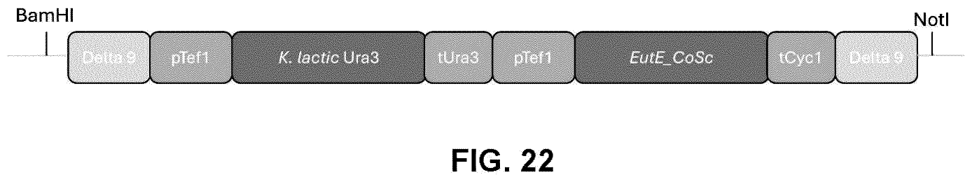

Referring now to FIG. 22 an exemplary embodiment of a sequence construct for EutE expression in a yeast strain with a recyclable K. lactic Ura3 is illustrated. Strain CJYL-145, the base strain with EutE heterologous expression was constructed by standard lithium acetate transformation with PCR amplification of the construct shown in FIG. 22 from plasmid OE-EutE with primers PBO_Prim_264 (CAGTGTAAAATGACGACATAATTGATGGGAAACAG) and PBO_Prim_265 (ACAGAATGTACCAAAAGTTATCCTGTAGGATATAGG) and transforming via LiAc transformation into CJYL-53. Transformants were selected on SD-URA. The resulting strain (CJYL-166) was transformed with pPBO101 to add the final step in the MVA pathway required for isoprene production. Transformants were selected on SD-URA+G418.

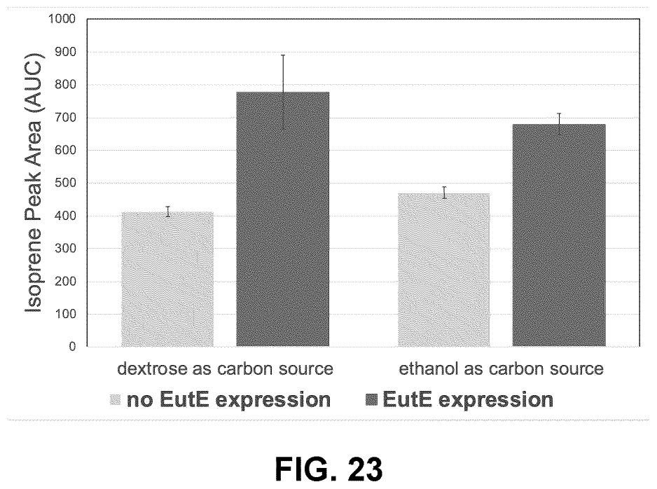

Referring now to FIG. 23, an exemplary experiment results of production of strains with or without EutE expression with dextrose or ethanol as the carbon source is illustrated. The x-axis represents yeast strains with or without EutE expression, with both strain types cultured on dextrose or ethanol as the carbon source. The y-axis represents isoprene production, measured by peak area (AUC). Heterologous expression of E. coli EutE under the control of the pTef1 promoter enhances isoprene production by approximately two- to four-fold on both dextrose and ethanol carbon sources. Additionally, the yeast strains are able to utilize ethanol as a carbon source and produce isoprene at levels comparable to those observed with dextrose.

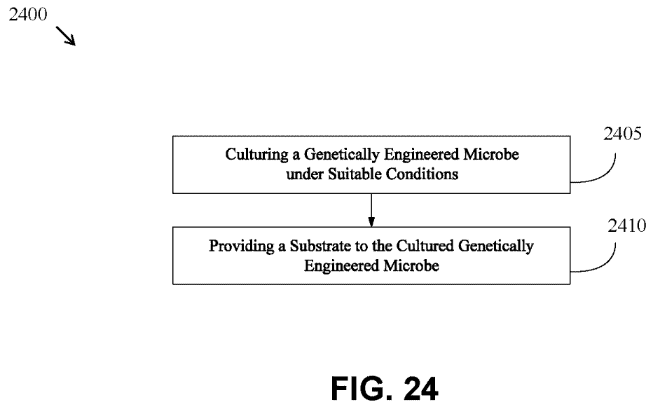

Referring now to FIG. 24, FIG. 24 includes a scheme of a method 2400 for producing isoprene using a genetically engineered microbe. Method 2400 may utilize ethanol as a carbon source. Method 2400 may utilize glucose as a carbon source. Method 2400 may utilize sucrose as a carbon source. Method 2400 may utilize a combination of ethanol and glycerol as a carbon source. At step 2405, method 2400 includes culturing a genetically engineered microbe under suitable conditions, consistent with details described above without limitation. For the purposes of this disclosure, a “suitable condition” is an environmental condition or factor suitable for the growth and/or replication of a microbe. In some cases, a suitable condition may vary from one type of microbe to another. A suitable condition may include without limitation a suitable temperature/temperature range, a suitable pressure/pressure range, a suitable pH or pH range, a suitable ionic strength/range of ionic strength, a suitable osmotic pressure/range of osmotic pressure, a suitable concentration/concentration range of one or more nutrients, a suitable level of metabolic waste/metabolites, among others. A person of ordinary skill in the art, upon reviewing the entirety of this disclosure, will be able to identify suitable conditions specific to one or more microbes described herein.

With continued reference to FIG. 24, at step 2410, method further includes providing substrate to genetically engineered microbe to produce isoprene, consistent with details described above without limitation. Specifically, genetically engineered microbe includes first nucleic acid sequence encoding first an enzyme, wherein the first enzyme is configured to catalyze one or more steps of a conversion from ethanol to A-CoA, a second nucleic acid sequence encoding a second enzyme of the MVA pathway, and a heterologous nucleic acid sequence encoding a third enzyme, wherein the third enzyme is configured to catalyze an isoprene-producing chemical reaction. In some cases, produced isoprene may be isolated and/or purified using an absorbing/stripping apparatus, consistent with details disclosed in U.S. patent application Ser. No. 18/928,691 (attorney docket number 1656-001USU1), filed on Oct. 28, 2024, entitled “SYSTEM AND METHOD FOR PRODUCING A DIMETHYLCYCLOOCTANE-BASED AVIATION FUEL FROM ISOPRENE”, the entirety of which is incorporated herein by reference.

### EXAMPLES

### Example 1: Create a New Strain of S. cerevisiae Capable of Producing Isoprene with Ethanol as the Carbon Source

### Significance

Lab strain of yeast (CJYL-7) engineered to reproducibly produce isoprene from sugars and ethanol.

### Background

Ethanol could be utilized as a carbon source (as opposed to a by-product of glucose metabolism) to shunt carbon to acetyl-CoA (and therefore isoprene). While E. coli has been engineered to utilize ethanol as a carbon source, the bacteria cannot natively utilize ethanol as a sole carbon source, nor are engineered strains particularly robust in consuming the alcohol (Cao et al 2020). On the other hand, the yeast Saccharomyces cerevisiae is naturally capable of undergoing a diauxic shift in the absence of glucose to consume ethanol as a primary carbon source (Galdieri et al 2010). Glycerol, a natural byproduct that yeast generates during fermentation of sugars into ethanol, is often included alongside ethanol to maintain osmotic equilibrium. Glycerol is less preferentially consumed by S. cerevisiae, with many variants unable to timely consume it as the sole carbon source (Swinnen et al 2013).

This report details CleanJoule's onboarding of isoprene production into a lab strain of yeast (BY4742), and subsequent demonstration of successful production of isoprene utilizing ethanol as the primary carbon source.

### Results

A strain of yeast was first constructed with two chromosomal integrations: pYI33 at the Delta22 site, and pYI31 and pYI32 at the Delta 15 site. pYI33 introduced a single copy each of pPDC1SC-ERG10SC-tADH1SC, pYEF3SC-ERG13SC-tTPS1SC, and pLEU2SC-LEU2SC-tLEU2SC. pY131 and pY132 introduced a single copy each of pRPL3SC-ERG12SC-tTPS1SC, pACT1SC-ERG8SC-tPRM9SC, pTEF2SC-MVD1SC-tCYC1SC, pRPL15aSC-IDI1SC-tSPO1SC, and pLYS2SC-LYS2SC-tLYS2SC (using a split LYS2 fragment on both DNA fragments for their simultaneous integration). This base strain was then transformed with 2μ URA plasmids expressing a variant of Populus alba IspS (either with F340L & A570N mutations relative to the wild type sequence, “IspSm”, or a truncated version of the wild type sequence with a 52 amino acid N-terminal portion removed and two different mutations S339C and G532S, “IspSf”) under control of either the ADH2 or TDH3 promoter, or the negative control pRS426 plasmid (FIG. 6). Notably, these strains do not have overexpression of HMG1. An alignment of the IspS protein sequences is shown in FIG. 7. Separately, the base strain was transformed with CEN G418 plasmids expressing both truncated yeast HMG1 (tHMG1) and various IspS sequences: P. alba IspS with two mutations (F340L and A570N) (pPBO017), the same IspS codon optimized differently (pPBO049) or His-tagged (pPBO050), and an IspS from the Kudzu vine Pueraria montana (pPBO048). All strains are outlined in FIG. 6.

Strains listed in FIG. 6 were assayed in five different Yeast Isoprene Production Screen (or YIPS) experiments (YIPS_2 to YIPS_6, with YIPS_1 being a previous MVA supplementation test). In YIPS_2, only the 2μ URA plasmids were assayed, (CJYL-4 through 8) using SD-URA minimal media supplemented with 20 g/L glucose. As our yeast produces ethanol, a longer GC protocol was used to separate the two peaks. Using this protocol, the ethanol peak appears at 6.5 minutes while the isoprene peak appears around 7.0 minutes.

FIG. 8 demonstrates CJYL-6 (the strain harboring pIspSm5-5) and CJYL-7 (the strain harboring p11-15) produced a consistent and measurable amount of a compound that aligns precisely with the expected peak for isoprene. As this peak does not appear in the negative control, the likeliest explanation is that this peak represents isoprene produced from yeast.

Additionally, strains CJYL-5 and CJYL-8 utilize the ADH2 promoter to drive IspS expression. It is expected that this promoter will be repressed in the presence of glucose and activated in the presence of ethanol. Therefore, it is not surprising that these strains did not produce isoprene in this experiment.

In YIPS_3, the carbon source was switched from glucose to 20 g/L sucrose. Additionally, the CEN G418 plasmids were also included, using SD minimal media without leucine or lysine and with G418 supplementation. In this run, measurable isoprene was again detected in CJYL-6 and CJYL-7, with the latter this time producing the most (FIG. 10). Additionally, strains CJYL-10 (with the P. montana IspS) and CJYL-11 (with new codon optimization of the mutant P. alba IspS) also produced measurable isoprene at similar trace levels to CJYL-6.

The final ODs were more consistent this time, indicative of cultures reaching saturation (FIG. 11). Peak areas were roughly equivalent to the previous run. Notably CJYL-10 and CJYL-11 with CEN G418 plasmids produced much less isoprene than CJYL-7, despite only the former strains having tHMG1 overexpression on their plasmids.

In YIPS_4, the carbon source was switched to either 50 g/L glucose, or ethanol/glycerol (at 4% and 3% volume respectively). Synthetic defined media was still used for all samples. Likely due to the higher sugar content and spiked ethanol, the final ethanol peaks were larger and bled more into the isoprene peak, necessitating a manual peak-skimming integration to correctly quantify peaks. As FIG. 12 shows, isoprene was still detectable in many samples.

A substantial isoprene peak was detected in CJYL-7 strain. This is indicative that isoprene was likely being produced from ethanol, as it is preferentially consumed over glycerol (FIG. 13).

To confirm that isoprene could be produced in ethanol+glycerol mixtures, the experiment was repeated in YIPS_5. In this experiment, strains with the WT and CEN G418 plasmids were grown in YP instead of SD minimal media (strains with the 2μ URA plasmids were kept in SD-Ura to maintain selective pressure on the plasmids). Additionally, the seed train included an adaptation step to 3% glycerol before dilution into either 3% glycerol or 3% glycerol+3% ethanol media in sealed GC vials (FIGS. 14A-B).

In this case, only CJYL-7 produced detectable isoprene. There was little growth of strains with the 2μ plasmids (CJYL-4, 6, 7) in the glycerol only condition, suggesting that these strains were unable to consume glycerol as the primary carbon source when grown in minimal media (as the strains grew in YPEG). There was detectable isoprene in the glycerol only condition for CJYL-7. Conversely, all strains grew to similar ODs in the ethanol/glycerol condition (in either YP or SD media). Furthermore, the isoprene peak of CJYL-7 was more substantial, suggesting that the yeast was consuming ethanol and converting it into isoprene (FIG. 15).

To confirm that CJYL-7 utilized ethanol rather than glycerol to produce isoprene, YIPS_6 was executed to replicate YIPS_5 with the addition of HPLC analysis of final culture to quantify final glycerol and ethanol concentrations. An additional day was included for growth of starter cultures (in glucose media, from 1 day to 2 days), as well as an additional day for growth of seed cultures (in glycerol media, from 2 days to 3 days). Final production cultures were sampled a day early (from 4 days to 3 days) as no significant growth was observed on the fourth day in YIPS_5 (from qualitative visual inspection). Further, a triplicate blank media was included to serve as process controls for glycerol and ethanol concentrations over time without consumption. Again, more than 10-fold less isoprene was produced in the glycerol only media (FIG. 16).

After isoprene titers and ODs were measured, culture media was collected, filter sterilized to remove residual cells and analyzed via HPLC to determine final glycerol and ethanol levels. Standard curves for Glycerol and Ethanol produced a strong correlation across the range evaluated (0.375%-6.0% Vol, R²=0.99999 and R²=0.99996 for Glycerol and Ethanol respectively). The change in glycerol was the same for both strains in both media, while more ethanol appeared to be consumed than glycerol in the mixed carbon source media. This low consumption of glycerol relative to ethanol coupled with the >10-fold isoprene titers in ethanol/glycerol media strongly suggests that ethanol is the major carbon source for CJYL-7's production of isoprene in YIPS_4-6 (FIG. 17). A compilation of all Isoprene peak areas identified across these experiments is reported in FIG. 18.

### Conclusion

Yeast strains were engineered with genomic integrations of key overexpression steps of the MVA pathway and were subsequently demonstrated to produce isoprene in various media. While multiple strains produced isoprene, the best strain in this set of experiments is CJYL-7, a strain harboring a 2μ plasmid with a URA marker and expression of a truncated P. alba IspS under the TDH3 promoter. This strain demonstrated production in SD media supplemented with glucose or ethanol and glycerol, but not in glycerol alone media. While the strain was able to grow in SD glycerol in the seed train in culture tubes with oxygen exposure, they were unable to grow in the same media in capped GC-vials in SD glycerol media.

These results demonstrate that isoprene can be produced from ethanol in yeast grown with ethanol+glycerol as the carbon source.

### Materials and Methods

### Cloning

Plasmid pYI31 was synthesized in house via Gibson assembly (as pPBO001). Plasmid pRS426 was purchased from ATTC. Plasmid pYI32 (AKA pPBO010), pYI33 (AKA pPBO012), pPBO017, and pPBO048-050 were synthesized by Genscript. The four 2μ URA plasmids were built internally using traditional and Gibson cloning methods from pRS426.

### Strain Construction

DNA for integration into the yeast genome was digested and gel purified to recover only the desired DNA for insertion. This DNA or DNA for plasmid expression was then transformed using standard lithium acetate protocols into BY4742 (or in-house strains of its lineage), selected on the appropriate selective media, restruck at least once on fresh selective media, grown in 3-5 mL minimal media with appropriate selection, and saved as glycerol stocks. Glycerol stocks were struck onto either YPD with G418 selection, SD, or SD-URA plates to begin isoprene production screens. All growth was performed at 30° C. (with 250 rpm when appropriate).

### Isoprene Production

For YIPS_2-5 (Yeast Isoprene Production Screen), strains were struck from glycerol stocks onto selective media and 3 individual colonies were grown in the appropriate liquid starter culture media at 30° C. and 250 RPM for approximately 48 hours. At the end of the culture period, ODs were typically between 10-15 in rich media and 5-10 in minimal media. For YIPS 5, an additional seed culture step was included. At this point, cells were diluted into the appropriate seed culture media as to a starting OD of 0.05 (YIPS_2 and YIPS_3) or a 1:100 dilution (YIPS_4 and YIPS_5) in a final volume of 3 mL in a GC Autosampler vial. The vials were sealed and incubated at 30° C. and 250 RPM for 2-4 days, at which point the headspace from the tubes was automatically sampled and analyzed by GC-FID with the HS_Isoprene_Ethanol_Long method (see below for details). Controls include pure isoprene, pure ethanol, and a mix of isoprene and ethanol in the same vial. The isoprene and ethanol controls were prepared by sampling the cold liquid phase of each directly via a 1 μL glass capillary. The capillaries were deposited into the associated control GC vial and sealed immediately. After the run, the vials were opened with the de-crimper tool and the final ODs reported were measured via spectrophotometer. FIG. 19 details all YIPS experiments and their associated experimental conditions, including strains evaluated and carbon sources for both starter and seed cultures. SD=synthetic defined complete (20 g/L glucose unless stated otherwise) and noted if amino acids are excluded, YPD=yeast peptone 20 g/L glucose, YPG=yeast peptone 3% (wt/vol) glycerol, YPEG=yeast peptone 3% (wt/vol) ethanol and 3% (wt/vol) glycerol.

### GC Analysis

Headspace gas chromatography analysis was performed using the HS_Isoprene_Ethanol_Long method under the following conditions. The autosampler oven was maintained at 35° C., with the sample loop and transfer line held at 40° C. and 45° C., respectively. Samples were introduced in “fill to pressure” mode, targeting a pressure of 15 psi. The gas chromatograph oven was initially held isothermally at 30° C. for 7.5 minutes, followed by a temperature ramp to 100° C. over 1.4 minutes, and subsequently held isothermally at 100° C. for an additional 1.1 minutes. The total run time was 10 minutes, with a constant carrier gas flow rate of 0.5 mL/min maintained throughout the analysis.

### HPLC Analysis

Samples were analyzed using an Agilent HPLC 1260 Infinity II system equipped with an Aminex™ HPX-87H column and using 5 mM H2SO4 as the mobile phase. The column was maintained at 60° C. while the RID module was maintained at 35° C. with a flow rate of 0.6 mL/min and total runtime of 30 minutes between samples. For standards, 6% glycerol and 6% ethanol was prepared in H₂O and used to create 5 serial dilutions. Samples were collected after GC analysis, spun to remove cells, filter sterilized, and 5 μL injected.

### Example 2: Improve S. cerevisiae's Isoprene Production Using Ethanol as a Carbon Source with Heterologous Expression of Acetaldehyde Dehydrogenase (A-ALD)

### Significance

Introduction of A-ALD into yeast by heterologous expression of E. coli A-ALD (EutE) gene improved isoprene production by 2-4 fold in comparison to the base strain. Additionally all strains can utilize ethanol as a carbon source for isoprene production, and expression of E. coli A-ALD (EutE) improved isoprene with ethanol as the carbon source as well.

### BACKGROUND

Isoprene biosynthesis via the mevalonate pathway depends on acetyl-coenzyme A (acetyl-CoA) as a major precursor. In Saccharomyces cerevisiae, previous studies have shown that increasing cytosolic acetyl-CoA availability enhances the production of isoprenoids. The native pathway for cytosolic acetyl-CoA synthesis in yeast is via the pyruvate dehydrogenase bypass, which involves three steps (see FIG. 21):
Pyruvate→Acetaldehyde (via pyruvate decarboxylase, PDC)
Acetaldehyde→Acetate (via acetaldehyde dehydrogenases, ALDH)
Acetate→Acetyl-CoA (via acetyl-CoA synthetases, ACS)

This final step, catalyzed by ACS1 and ACS2, requires ATP, making it energetically costly. Therefore, ATP-independent alternative pathways could improve the energy efficiency of acetyl-CoA production.

One such alternative is the use of acetylating acetaldehyde dehydrogenase (A-ALD)—an enzyme found in many prokaryotes that directly converts acetaldehyde to acetyl-CoA in an ATP-independent manner. Prior work has shown that heterologous expression of bacterial A-ALD enzymes in yeast can replace or complement the native ACS-based pathway.

In yeast another pathway to enter the mevalonate pathway is through utilizing ethanol as a carbon source. The reaction from ethanol to acetyl-CoA in yeast is catalyzed by 3 enzymatic steps (see FIG. 21):
Alcohol→Acetaldehyde (via alcohol dehydrogenase, ADH)
Acetaldehyde→Acetate (via acetaldehyde dehydrogenases, ALDH)
Acetate→Acetyl-CoA (via acetyl-CoA synthetases, ACS)

The introduction of A-Ald using ethanol as an alternative carbon source can also be leveraged with the following pathway (see FIG. 21):
Ethanol→Acetaldehyde→Acetyl-CoA, to further support ATP-efficient acetyl-CoA synthesis.

Therefore, heterologous expression of EutE was introduced via an A-ALD from Escherichia coli to increase the acetyl-CoA pool and consequently increase isoprene production in S. cerevisiae. Subsequent experiments demonstrate again that ethanol can be utilized as a carbon source to synthesize isoprene.

### Results

TABLE 2
Yeast strains used in this study.
CJYL = CleanJoule Yeast Laboratory.

| | | | | Plasmid |
|---|---|---|---|---|
| Strain | Genotype | Plasmid | Marker | DNA |
| CJYL-53 | ↑MVA + 2xtHMG1 | NONE | NONE | NONE |
| CJYL-107 | ↑MVA + 2xtHMG1 | pPBO101 | G418 | IspS(LN) |
| CJYL-145 | ↑MVA + 2xtHMG1 + EutE | NONE | NONE | NONE |
| CJYL-166 | ↑MVA + 2xtHMG1 + EutE | pPBO101 | G418 | IspS(LN) |

* ↑MVA base strain has the following genomic modifications in BY4742 parental strain: pPDC1SC-ERG10SC-tADH1SC + pYEF3SC-ERG13SC-tTPS1SC + pLEU2SC-LEU2SC-tLEU2SC + pRPL3SC-ERG12SC-tTPS1SC + pACT1SC-ERG8SC-tPRM9SC + pTEF2SC-MVD1SC-tCYC1SC + pRPL15aSC-IDI1SC-tSPO1SC + pLYS2SC-LYS2SC-tLYS2SC

Strain CJYL-145, the base strain with EutE heterologous expression was constructed by standard lithium acetate transformation with PCR amplification of the construct shown in FIG. 22 from plasmid OE-EutE with primers PBO_Prim 264 (CAGTGTAAAATGACGACATAATTGATGGGAAACAG) and PBO_Prim_265 (ACAGAATGTACCAAAAGTTATCCTGTAGGATATAGG) and transforming via LiAc transformation into CJYL-53. Transformants were selected on SD-URA. The resulting strain (CJYL-166) was transformed with pPBO101 to add the final step in the MVA pathway required for isoprene production. Transformants were selected on SD-URA+G418.

Whole genome sequencing (Oxford Nanopore long-read sequencing) was used to confirm heterologous expression of E. coli EutE in S. cerevisiae. Genomic DNA was prepared using the Zymo YeaStar Genomic DNA Kit.

Heterologous expression of E. coli EutE under the control of pTef1 enhances isoprene production between two to four-fold on dextrose or ethanol as the carbon source. Additionally, the yeast strains can utilize ethanol as a carbon source and produce similar amounts of isoprene as with dextrose (FIG. 23).

### Materials and Methods

### Cloning

The plasmid containing OE::EutE was constructed at Genscript. Briefly, EutE sequence from E. coli was codon optimized for yeast using IDT's codon optimization tool driven by Tef1 promoter and Cyc1 terminator of S. cerevisiae. Additionally, a URA3 recyclable marker from Kluyveromyces lactis driven by Ura3 (p) was constructed to be integrated into the delta 9 site.

### Strain Construction

DNA for integration into the yeast genome was generated by PCR. This DNA or DNA for plasmid expression was then transformed using standard lithium acetate protocols into (or in-house strains of its lineage), selected on the appropriate selective media, restruck at least once on fresh selective media, grown in 3-5 mL minimal media with appropriate selection, and saved as glycerol stocks. Glycerol stocks were struck onto either YPD with G418 selection and SD-URA+G418 plates to begin isoprene production screens. All growth was performed at 30° C. (with 250 rpm when appropriate).

### Isoprene Production

To test for isoprene production, a 10 uL loop of the respective yeast strains were inoculated into 50 mL of Yeast Peptone Dextrose (20 g/L)+G418 as the preculture. These precultures were grown in 250 mL baffled flask 30° C. and 250 RPM for approximately 24 hours. The cells were spun down, washed twice in sterile DI H20, and an OD600 of 5 were inoculated into the appropriate production culture media* in a final volume of 2 mL in a GC Autosampler vial. The vials were sealed and incubated at 30° C. and 250 RPM for 2 days, at which point the headspace from the tubes was automatically sampled and analyzed by GC-FID with the HS_Isoprene_method (see below for details). * The production culture media were YPD+G418 and YPEG (Yeast Peptone Glycerol (4% glycerol) Ethanol (3% ethanol))+G418.

### GC Analysis

Analysis was performed using a ResTEK Rxi-624Sil column installed on an Agilent 8890 GC System equipped with a 7697A Headspace Autosampler, following the method designated “2025 Jul. 29 Isoprene Ethanol ResTEK.” The autosampler oven temperature was maintained at 100° C., the sample loop temperature at 100° C., and the transfer line temperature at 115° C., with vial shaking set to 18 shakes per minute. The autosampler operated in fill-to-pressure mode at 15 psi. The GC inlet temperature was set to 300° C., with an inlet pressure of 11.324 psi and operated in split mode at a ratio of 100:1 with a total flow of 500 mL/min. The column flow was maintained at 5 mL/min. The GC oven was held at 60° C., with the program running for a total of 4 minutes, including an isothermal hold at 60° C. for 3 minutes, with a total runtime of 3 minutes. The detector was set at 300° C. with air flow at 400 mL/min, hydrogen flow at 40 mL/min, and nitrogen makeup flow at 30 mL/min.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A genetically engineered microbe capable of producing isoprene from a carbon source, the genetically engineered microbe comprising:
a first nucleic acid sequence encoding a first enzyme, wherein the first enzyme is configured to catalyze one or more steps of a conversion from ethanol to acetyl coenzyme A (A-CoA);
a second nucleic acid sequence encoding a second enzyme of a mevalonate (MVA) pathway; and
a heterologous nucleic acid sequence encoding a third enzyme, wherein the third enzyme is configured to catalyze an isoprene-producing chemical reaction.

2. The genetically engineered microbe of claim 1, wherein the first nucleic acid sequence includes one or more members selected from a group consisting of ADH (alcohol dehydrogenase) gene, ALD (acetaldehyde dehydrogenase) gene, ACS (acetyl coenzyme A synthetase) gene, and (acylating acetaldehyde dehydrogenase) A-ALD gene.

3. The genetically engineered microbe of claim 1, wherein the first enzyme includes one or more members selected from a group consisting of alcohol dehydrogenase (ADH), acetaldehyde dehydrogenase (ALD), acetyl coenzyme A synthetase (ACS), and acylating acetaldehyde dehydrogenase (A-ALD).

4. The genetically engineered microbe of claim 1, wherein the first nucleic acid sequence is A-ALD (acylating acetaldehyde dehydrogenase) gene and an expression of native ACS1 and ACS2 genes is eliminated.

5. The genetically engineered microbe of claim 1, wherein the first enzyme is an acylating acetaldehyde dehydrogenase, wherein a native acetyl coenzyme A synthetase activity is eliminated.

6. The genetically engineered microbe of claim 1, wherein the second nucleic acid sequence includes one or more members selected from a group consisting of ERG10 gene, ERG13 gene, HMG1, a truncated HMG1 gene (tHMG1), HMG2 gene, a truncated HMG2 gene (tHMG2), MvaE gene, ERG12 gene, ERG8 gene, MVD1 gene, and IDI1 gene.

7. The genetically engineered microbe of claim 1, wherein the second enzyme includes one or more members selected from a group consisting of 3-hydroxy-3-methylglutaryl-coenzyme A reductase, 3-hydroxy-3-methylglutaryl-CoA synthase, HMG-COA reductase, mevalonate kinase, phosphomevalonate kinase, diphosphomevalonate decarboxylase, and isopentenyl diphosphate isomerase.

8. The genetically engineered microbe of claim 1, wherein the third enzyme comprises at least a functional fragment of isoprene synthase (IspS).

9. The genetically engineered microbe of claim 8, wherein the functional fragment of isoprene synthase (IspS) comprises a polypeptide having at least one amino acid substitution selected from the group consisting of F340L and A570N.

10. The genetically engineered microbe of claim 8, wherein the functional fragment of isoprene synthase (IspS) comprises a polypeptide having at least one amino acid substitution selected from the group consisting of S339C and G542S.

11. The genetically engineered microbe of claim 8, wherein the functional fragment of isoprene synthase (IspS) comprises a polypeptide having at least 52 amino acids removed from N-terminal portion of the polypeptide.

12. The genetically engineered microbe of claim 1, wherein the isoprene-producing chemical reaction utilizes dimethylallyl pyrophosphate (DMAPP) as a reactant.

13. The genetically engineered microbe of claim 1, wherein the genetically engineered microbe comprises fungi.

14. The genetically engineered microbe of claim 13, wherein the fungi comprise Saccharomyces cerevisiae, Ogataea polymorpha, Yarrowia lipolytica, or Aspergillus.

15. The genetically engineered microbe of claim 1, wherein the genetically engineered microbe comprises bacteria.

16. The genetically engineered microbe of claim 15, wherein the bacteria comprise acetic acid bacteria (AAB).

17. The genetically engineered microbe of claim 16, wherein the AAB includes one or more members selected from a group consisting of Acetobacter including Acetobacter aceti, Acetobacter cerevisiae, Acetobacter malorum, Acetobacter oeni, Acetobacter pasteurianus, Acetobacter pomorum, Acetobacter peroxydans, Acetobacter diazotrophicu, Acetobacter europaeus, Acetobacter hansenii, Acetobacter liquefaciens, Acetobacter xylinus, Acetobacter cibinongensis, Acetobacter estunensis, Acetobacter indonesiensis, Acetobacter nitrogenifigens, Acetobacter orientalis, Acetobacter orleanensis, Acetobacter tropicalis, Acetobacter lovaniensis, and Acetobacter syzygii, Acidomonas, Ameyamaea, Asaia, Bombella, Commensalibacter, Endobacter, Gluconacetobacter including Gluconacetobacter entanii, Gluconacetobacter liquefaciens, Gluconacetobacter azotocaptans, Gluconacetobacter diazotrophicus, Gluconacetobacter sacchari, Gluconacetobacter europaeus, Gluconacetobacter hansenii, Gluconacetobacter intermedius, Gluconacetobacter nataicola, Gluconacetobacter oboediens, Gluconacetobacter rhaeticus, Gluconacetobacter saccharivorans, Gluconacetobacter swingisii, and Gluconacetobacter xylinus, Gluconobacter including Gluconobacter oxydans, Gluconobacter albidus, Gluconobacter cerinus, and Gluconobacter frateurii, Granulibacter, Komagataeibacter including Komagataeibacter europaeus, Komagataeibacter hansenii, Komagataeibacter intermedius, Komagataeibacter medellinensis, Komagataeibacter oboediens, and Komagataeibacter xylinus, Kozakia, Neoasaia, Neokomagataea, Nguyenibacter, Saccharibacter, Swaminathania, Swingsia, and TanticharoeniAcetobacter.

18. The genetically engineered microbe of claim 1, wherein the genetically engineered microbe comprises a Crabtree-positive microbe.

19. The genetically engineered microbe of claim 1, wherein the genetically engineered microbe comprises a diploid strain.

20. The genetically engineered microbe of claim 1, wherein the genetically engineered microbe has been modified such that an activity of the first enzyme or the second enzyme is modulated by increasing an expression of the first nucleic acid sequence or the second nucleic acid sequence.

21. The genetically engineered microbe of claim 1, wherein the genetically engineered microbe has been modified such that an activity of a fourth enzyme is modulated by decreasing, attenuating, or deleting an expression of a fourth nucleic acid sequence encoding the fourth enzyme.

22. The genetically engineered microbe of claim 21, wherein the fourth nucleic acid sequence includes one or more members selected from a group consisting of a BTS1 gene, an ERG20 gene, a PDH gene, an AceE gene, a poxB gene, a PDC gene, an AceB gene, an MLS1 gene, a CIT1 gene, an ADH1 gene, an adhE gene, and Idha gene, a did gene, an ackA gene, and a NudB gene.

23. The genetically engineered microbe of claim 21, wherein the fourth enzyme comprises one or more members selected from a group consisting of geranyl diphosphate synthase (IspA), geranyl diphosphate synthase (GPPS), farnesyl pyrophosphate synthetase (Erg 20), geranyl geranylgeranyl diphosphate synthase (BTS1), farnesyl diphosphate synthase (FPPS), pyruvate dehydrogenase (PDH) including pyruvate dehydrogenase E1 component, pyruvate oxidase, pyruvate decarboxylase (PDC), malate synthase (AceB), citrate synthase (CIT1), ATP-citrate lyase (CitE) including AclY, Ac1B, and Ac1A, glycerol-producing enzymes including cGPDH and G3PP [41], ethanol-producing enzymes including invertase and zymase, alcohol dehydrogenase (ADH1), alcohol dehydrogenase enzyme (adhE), lactate-producing enzymes, lactate dehydrogenase including lactate dehydrogenase A (IdhA) and D-lactate dehydrogenase (did), acetate-producing enzymes including acetyl coenzyme A (acetyl-CoA) hydrolases, acetate kinase (encoded by ackA), phosphatase, and hydrolase.

24. The genetically engineered microbe of claim 21, wherein disrupting expression of a fourth nucleic acid sequence encoding the fourth enzyme comprises deleting an upstream activation sequence in a promoter of squalene synthase (ERG9), or the nucleic acid sequence encoding peroxisomal membrane E3 ubiquitin ligase (PEX10).

25. The genetically engineered microbe of claim 1, wherein the genetically engineered microbe is configured to produce an agent, wherein the agent is configured to inhibiting a phosphotransacetylase-acetate kinase (Pta-AckA) pathway, thereby facilitating an acetate reuptake.

26. The genetically engineered microbe of claim 1, wherein the genetically engineered microbe further comprises a fifth nucleic acid sequence encoding a fifth enzyme.

27. The genetically engineered microbe of claim 26, wherein the fifth nucleic acid sequence comprises Alcohol Dehydrogenase 2 (ADH2) gene, Triosephosphate Dehydrogenase 3 (TDH3) gene, or Pyruvate Decarboxylase (PDC1 gene).

28. A method of producing isoprene from carbon source, the method comprising:
culturing a genetically engineered microbe under suitable conditions, the genetically engineered microbe comprising:
a first nucleic acid sequence encoding a first enzyme, wherein the first enzyme is configured to catalyze one or more steps of a conversion from ethanol to acetyl coenzyme A-CoA;
a second nucleic acid sequence encoding a second enzyme of an MVA pathway; and
a heterologous nucleic acid sequence encoding a third enzyme, wherein the third enzyme is configured to catalyze an isoprene-producing chemical reaction; and
providing a substrate to the cultured genetically engineered microbe.

29. The method of claim 28, wherein the first nucleic acid sequence includes one or more members selected from a group consisting of ADH (alcohol dehydrogenase) gene, ALD (acetaldehyde dehydrogenase) gene, ACS (acetyl coenzyme A synthetase) gene, and (acylating acetaldehyde dehydrogenase) A-ALD gene.

30. The method of claim 28, wherein the first enzyme includes one or more members selected from a group consisting of alcohol dehydrogenase (ADH), acetaldehyde dehydrogenase (ALD), acetyl coenzyme A synthetase (ACS), and acylating acetaldehyde dehydrogenase (A-ALD).

31. The method of claim 28, wherein the first nucleic acid sequence is A-ALD (acylating acetaldehyde dehydrogenase) gene and an expression of native ACS1 and ACS2 genes is eliminated.

32. The method of claim 28, wherein the first enzyme is an acylating acetaldehyde dehydrogenase native acetyl coenzyme A synthetase activity is eliminated.

33. The method of claim 28, wherein the second nucleic acid sequence includes one or more members selected from a group consisting of ERG10 gene, ERG13 gene, HMG1, a truncated HMG1 gene (tHMG1), HMG2 gene, a truncated HMG2 gene (tHMG2), MvaE gene, ERG12 gene, ERG8 gene, MVD1 gene, and IDI1 gene.

34. The method of claim 28, wherein the second enzyme includes one or more members selected from a group consisting of 3-hydroxy-3-methylglutaryl-coenzyme A reductase, 3-hydroxy-3-methylglutaryl-CoA synthase, HMG-COA reductase, mevalonate kinase, phosphomevalonate kinase, diphosphomevalonate decarboxylase, and isopentenyl diphosphate isomerase.

35. The method of claim 28, wherein the third enzyme comprises at least a functional fragment of isoprene synthase (IspS).

36. The method of claim 35, wherein at least a functional fragment of isoprene synthase (IspS) comprises a polypeptide having at least one amino acid substitution selected from the group consisting of F340L and A570N.

37. The method of claim 35, wherein at least a functional fragment of isoprene synthase (IspS) comprises a polypeptide having at least one amino acid substitution selected from the group consisting of S339C and G542S.

38. The method of claim 35, wherein the functional fragment of isoprene synthase (IspS) comprises a polypeptide having at least 52 amino acids removed from N-terminal portion of the polypeptide.

39. The method of claim 28, wherein the isoprene-producing chemical reaction utilizes dimethylallyl pyrophosphate (DMAPP) as a reactant.

40. The method of claim 28, wherein the genetically engineered microbe comprises fungi.

41. The method of claim 40, wherein the fungi comprise Saccharomyces cerevisiae, Ogataea polymorpha, Yarrowia lipolytica, or Aspergillus.

42. The method of claim 28, wherein the genetically engineered microbe comprises bacteria.

43. The method of claim 42, wherein the bacteria comprise acetic acid bacteria (AAB).

44. The method of claim 43, wherein the AAB includes one or more members selected from a group consisting of Acetobacter including Acetobacter aceti, Acetobacter cerevisiae, Acetobacter malorum, Acetobacter oeni, Acetobacter pasteurianus, Acetobacter pomorum, Acetobacter peroxydans, Acetobacter diazotrophicu, Acetobacter europaeus, Acetobacter hansenii, Acetobacter liquefaciens, Acetobacter xylinus, Acetobacter cibinongensis, Acetobacter estunensis, Acetobacter indonesiensis, Acetobacter nitrogenifigens, Acetobacter orientalis, Acetobacter orleanensis, Acetobacter tropicalis, Acetobacter lovaniensis, and Acetobacter syzygii, Acidomonas, Ameyamaea, Asaia, Bombella, Commensalibacter, Endobacter, Gluconacetobacter including Gluconacetobacter entanii, Gluconacetobacter liquefaciens, Gluconacetobacter azotocaptans, Gluconacetobacter diazotrophicus, Gluconacetobacter sacchari, Gluconacetobacter europaeus, Gluconacetobacter hansenii, Gluconacetobacter intermedius, Gluconacetobacter nataicola, Gluconacetobacter oboediens, Gluconacetobacter rhaeticus, Gluconacetobacter saccharivorans, Gluconacetobacter swingisii, and Gluconacetobacter xylinus, Gluconobacter including Gluconobacter oxydans, Gluconobacter albidus, Gluconobacter cerinus, and Gluconobacter frateurii, Granulibacter, Komagataeibacter including Komagataeibacter europaeus, Komagataeibacter hansenii, Komagataeibacter intermedius, Komagataeibacter medellinensis, Komagataeibacter oboediens, and Komagataeibacter xylinus, Kozakia, Neoasaia, Neokomagataea, Nguyenibacter, Saccharibacter, Swaminathania, Swingsia, and TanticharoeniAcetobacter.

45. The method of claim 28, wherein the genetically engineered microbe comprises a Crabtree-positive microbe.

46. The method of claim 28, wherein the genetically engineered microbe comprises a diploid strain.

47. The method of claim 28, wherein the genetically engineered microbe has been modified such that an activity of the first enzyme or the second enzyme is modulated by increasing an expression of the first nucleic acid sequence or the second nucleic acid sequence.

48. The method of claim 28, wherein the genetically engineered microbe has been modified such that an activity of a fourth enzyme is modulated by decreasing, attenuating, or deleting an expression of a fourth nucleic acid sequence encoding the fourth enzyme.

49. The method of claim 48, wherein the fourth nucleic acid sequence wherein the fourth nucleic acid sequence includes one or more members selected from a group consisting of a BTS1 gene, an ERG20 gene, a PDH gene, an AceE gene, a poxB gene, a PDC gene, an AceB gene, an MLS1 gene, a CIT1 gene, an ADH1 gene, an adhE gene, and Idha gene, a did gene, an ackA gene, and a NudB gene.

50. The method of claim 48, wherein the fourth enzyme comprises one or more members selected from a group consisting of a group consisting of geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), pyruvate dehydrogenase (PDH) including pyruvate dehydrogenase E1 component, pyruvate oxidase, pyruvate decarboxylase (PDC), malate synthase (AceB), citrate synthase (CIT1), ATP-citrate lyase (CitE) including AclY, Ac1B, and Ac1A, glycerol-producing enzymes including cGPDH and G3PP [41], ethanol-producing enzymes including invertase and zymase, alcohol dehydrogenase (ADH1), alcohol dehydrogenase enzyme (adhE), lactate-producing enzymes, lactate dehydrogenase including lactate dehydrogenase A (IdhA) and D-lactate dehydrogenase (did), acetate-producing enzymes including acetyl coenzyme A (acetyl-CoA) hydrolases, acetate kinase (encoded by ackA), phosphatase, and hydrolase.

51. The method of claim 48, wherein deleting an expression of a fourth nucleic acid sequence encoding the fourth enzyme comprises deleting an upstream activation sequence in a promoter of squalene synthase (ERG9), or the nucleic acid sequence encoding peroxisomal membrane E3 ubiquitin ligase (PEX10).

52. The method of claim 28, wherein the genetically engineered microbe is configured to produce an agent, wherein the agent is configured to inhibiting a phosphotransacetylase-acetate kinase (Pta-AckA) pathway, thereby facilitating an acetate reuptake.

53. The method of claim 28, wherein the genetically engineered microbe further comprises a fifth nucleic acid sequence encoding a fifth enzyme.

54. The method of claim 53, wherein the fifth nucleic acid sequence comprises Alcohol Dehydrogenase 2 (ADH2) gene, Triosephosphate Dehydrogenase 3 (TDH3) gene, or Pyruvate Decarboxylase (PDC1 gene).
